SEQUENCE LISTING (582 sequences)

SEQ ID NO: 1 - DNA, 21 bp, synthetic construct
  misc_feature 1..21: note = siRNA constructs for CLEC16A
  source 1..21: mol_type = other DNA
caaaaaaatgtcttctccctt

SEQ ID NO: 2 - DNA, 21 bp, synthetic construct
  misc_feature 1..21: note = siRNA constructs for CLEC16A
  source 1..21: mol_type = other DNA
aaatatccaaaggagatgttt

SEQ ID NO: 3 - DNA, 21 bp, synthetic construct
  misc_feature 1..21: note = siRNA constructs for CLEC16A
  source 1..21: mol_type = other DNA
ctgctatagcagaaaaccatt

SEQ ID NO: 4 - DNA, 21 bp, synthetic construct
  misc_feature 1..21: note = siRNA constructs for CLEC16A
  source 1..21: mol_type = other DNA
ttctcttttattgccaagttt

SEQ ID NO: 5 - DNA, 21 bp, synthetic construct
  misc_feature 1..21: note = siRNA constructs for CLEC16A
  source 1..21: mol_type = other DNA
gaggttttctaaccctcggtt

SEQ ID NO: 6 - DNA, 21 bp, synthetic construct
  misc_feature 1..21: note = siRNA constructs for CLEC16A
  source 1..21: mol_type = other DNA
caaaacgtggtacagatactt

SEQ ID NO: 7 - DNA, 21 bp, synthetic construct
  misc_feature 1..21: note = siRNA constructs for CLEC16A
  source 1..21: mol_type = other DNA
ttctgtgactgtggtgttttt

SEQ ID NO: 8 - DNA, 21 bp, synthetic construct
  misc_feature 1..21: note = siRNA constructs for CLEC16A
  source 1..21: mol_type = other DNA
ctagcaggttccggttctgtt

SEQ ID NO: 9 - DNA, 21 bp, synthetic construct
  misc_feature 1..21: note = siRNA constructs for CLEC16A
  source 1..21: mol_type = other DNA
ctccactagcaggttccggtt

SEQ ID NO: 10 - DNA, 21 bp, synthetic construct
  misc_feature 1..21: note = siRNA constructs for CLEC16A
  source 1..21: mol_type = other DNA
gatggtctccactagcaggtt

SEQ ID NO: 11 - DNA, 21 bp, synthetic construct
  misc_feature 1..21: note = siRNA constructs for CLEC16A
  source 1..21: mol_type = other DNA
caagaagaaaacaaacatatt

SEQ ID NO: 12 - DNA, 21 bp, synthetic construct
  misc_feature 1..21: note = siRNA constructs for CLEC16A
  source 1..21: mol_type = other DNA
acacgtaacggcccgactttt

SEQ ID NO: 13 - DNA, 21 bp, synthetic construct
  misc_feature 1..21: note = siRNA constructs for CLEC16A
  source 1..21: mol_type = other DNA
tgaggtctcgtgactgatgtt

SEQ ID NO: 14 - DNA, 21 bp, synthetic construct
  misc_feature 1..21: note = siRNA constructs for CLEC16A
  source 1..21: mol_type = other DNA
ctcatcagaaaagtcaaattt

SEQ ID NO: 15 - DNA, 19 bp, synthetic construct
  misc_feature 1..19: note = siRNA constructs for CLEC16A
  source 1..19: mol_type = other DNA
gagttttaacgaaagtgtt

SEQ ID NO: 16 - DNA, 21 bp, synthetic construct
  misc_feature 1..21: note = siRNA constructs for CLEC16A
  source 1..21: mol_type = other DNA
aaagaaatggacagtgtggtt

SEQ ID NO: 17 - DNA, 21 bp, synthetic construct
  misc_feature 1..21: note = siRNA constructs for CLEC16A
  source 1..21: mol_type = other DNA
tgtgtacagggcaaagtcatt

SEQ ID NO: 18 - DNA, 21 bp, synthetic construct
  misc_feature 1..21: note = siRNA constructs for CLEC16A
  source 1..21: mol_type = other DNA
ggttgaaaaacttgatggctt

SEQ ID NO: 19 - DNA, 21 bp, synthetic construct
  misc_feature 1..21: note = siRNA constructs for CLEC16A
  source 1..21: mol_type = other DNA
ttcagggtggttgaaaaactt

SEQ ID NO: 20 - DNA, 21 bp, synthetic construct
  misc_feature 1..21: note = siRNA constructs for CLEC16A
  source 1..21: mol_type = other DNA
actttatagacattcaaagtt

SEQ ID NO: 21 - DNA, 21 bp, synthetic construct
  misc_feature 1..21: note = siRNA constructs for CLEC16A
  source 1..21: mol_type = other DNA
cccaatgaaccagaccaaatt

SEQ ID NO: 22 - DNA, 21 bp, synthetic construct
  misc_feature 1..21: note = siRNA constructs for CLEC16A
  source 1..21: mol_type = other DNA
tctgcacgcagtcatcgagtt

SEQ ID NO: 23 - DNA, 21 bp, synthetic construct
  misc_feature 1..21: note = siRNA constructs for CLEC16A
  source 1..21: mol_type = other DNA
gttgatgatcaggatgtcatt

SEQ ID NO: 24 - DNA, 21 bp, synthetic construct
  misc_feature 1..21: note = siRNA constructs for CLEC16A
  source 1..21: mol_type = other DNA
atcgttgaggaactcacagtt

SEQ ID NO: 25 - DNA, 21 bp, synthetic construct
  misc_feature 1..21: note = siRNA constructs for CLEC16A
  source 1..21: mol_type = other DNA
gtggtcagtgagcacatcgtt

SEQ ID NO: 26 - DNA, 21 bp, synthetic construct
  misc_feature 1..21: note = siRNA constructs for CLEC16A
  source 1..21: mol_type = other DNA
agcggtgcatgatgtataatt

SEQ ID NO: 27 - DNA, 21 bp, synthetic construct
  misc_feature 1..21: note = siRNA constructs for CLEC16A
  source 1..21: mol_type = other DNA
aatgacttcagctaacgagtt

SEQ ID NO: 28 - DNA, 21 bp, synthetic construct
  misc_feature 1..21: note = siRNA constructs for CLEC16A
  source 1..21: mol_type = other DNA
gatcaccattcagaatgactt

SEQ ID NO: 29 - DNA, 21 bp, synthetic construct
  misc_feature 1..21: note = siRNA constructs for CLEC16A
  source 1..21: mol_type = other DNA
catctcagacagatcaccatt

SEQ ID NO: 30 - DNA, 21 bp, synthetic construct
  misc_feature 1..21: note = siRNA constructs for CLEC16A
  source 1..21: mol_type = other DNA
ctgaatatcctgttcagtctt

SEQ ID NO: 31 - DNA, 21 bp, synthetic construct
  misc_feature 1..21: note = siRNA constructs for CLEC16A
  source 1..21: mol_type = other DNA
aacttctctgaatatcctgtt

SEQ ID NO: 32 - DNA, 21 bp, synthetic construct
  misc_feature 1..21: note = siRNA constructs for CLEC16A
  source 1..21: mol_type = other DNA
cctcttgcccttgtgcttgtt

SEQ ID NO: 33 - DNA, 21 bp, synthetic construct
  misc_feature 1..21: note = siRNA constructs for CLEC16A
  source 1..21: mol_type = other DNA
tgggccctttctcctcatctt

SEQ ID NO: 34 - DNA, 21 bp, synthetic construct
  misc_feature 1..21: note = siRNA constructs for CLEC16A
  source 1..21: mol_type = other DNA
ggcatcctcggtgggcccttt

SEQ ID NO: 35 - DNA, 21 bp, synthetic construct
  misc_feature 1..21: note = siRNA constructs for CLEC16A
  source 1..21: mol_type = other DNA
gctggattcgctctaattttt

SEQ ID NO: 36 - DNA, 21 bp, synthetic construct
  misc_feature 1..21: note = siRNA constructs for CLEC16A
  source 1..21: mol_type = other DNA
atctggctgggcagcgttgtt

SEQ ID NO: 37 - DNA, 21 bp, synthetic construct
  misc_feature 1..21: note = siRNA constructs for CLEC16A
  source 1..21: mol_type = other DNA
cagcgtcgccagccggatctt

SEQ ID NO: 38 - DNA, 21 bp, synthetic construct
  misc_feature 1..21: note = siRNA constructs for CLEC16A
  source 1..21: mol_type = other DNA
actcatcaggacttgctgctt

SEQ ID NO: 39 - DNA, 21 bp, synthetic construct
  misc_feature 1..21: note = siRNA constructs for CLEC16A
  source 1..21: mol_type = other DNA
agccagcactcatcaggactt

SEQ ID NO: 40 - DNA, 21 bp, synthetic construct
  misc_feature 1..21: note = siRNA constructs for CLEC16A
  source 1..21: mol_type = other DNA
gcaggccaggtgcacgtcctt

SEQ ID NO: 41 - DNA, 19 bp, synthetic construct
  misc_feature 1..19: note = siRNA constructs for CLEC16A
  source 1..19: mol_type = other DNA
acatgtccaaaaaaatgtc

SEQ ID NO: 42 - DNA, 21 bp, synthetic construct
  misc_feature 1..21: note = siRNA constructs for CLEC16A
  source 1..21: mol_type = other DNA
tcatgctcctatactcatctt

SEQ ID NO: 43 - DNA, 21 bp, synthetic construct
  misc_feature 1..21: note = siRNA constructs for CLEC16A
  source 1..21: mol_type = other DNA
aggcttgtggatggtgatgtt

SEQ ID NO: 44 - DNA, 21 bp, synthetic construct
  misc_feature 1..21: note = siRNA constructs for CLEC16A
  source 1..21: mol_type = other DNA
ctggtcaggaaggtgaggctt

SEQ ID NO: 45 - DNA, 21 bp, synthetic construct
  misc_feature 1..21: note = siRNA constructs for CLEC16A
  source 1..21: mol_type = other DNA
tgcgctcctggccacgttctt

SEQ ID NO: 46 - DNA, 21 bp, synthetic construct
  misc_feature 1..21: note = siRNA constructs for CLEC16A
  source 1..21: mol_type = other DNA
ggctgcgctcctggccacgtt

SEQ ID NO: 47 - DNA, 21 bp, synthetic construct
  misc_feature 1..21: note = siRNA constructs for CLEC16A
  source 1..21: mol_type = other DNA
tgggtgctcccagtgtgtctt

SEQ ID NO: 48 - DNA, 21 bp, synthetic construct
  misc_feature 1..21: note = siRNA constructs for CLEC16A
  source 1..21: mol_type = other DNA
ggagcgcagggataggtggtt

SEQ ID NO: 49 - DNA, 21 bp, synthetic construct
  misc_feature 1..21: note = siRNA constructs for CLEC16A
  source 1..21: mol_type = other DNA
tgatgtttttggacttctttt

SEQ ID NO: 50 - DNA, 21 bp, synthetic construct
  misc_feature 1..21: note = siRNA constructs for CLEC16A
  source 1..21: mol_type = other DNA
gttgatgtttttggacttctt

SEQ ID NO: 51 - DNA, 21 bp, synthetic construct
  misc_feature 1..21: note = siRNA constructs for CLEC16A
  source 1..21: mol_type = other DNA
agtgttgatgtttttggactt

SEQ ID NO: 52 - DNA, 21 bp, synthetic construct
  misc_feature 1..21: note = siRNA constructs for CLEC16A
  source 1..21: mol_type = other DNA
aaccttagtgttgatgttttt

SEQ ID NO: 53 - DNA, 21 bp, synthetic construct
  misc_feature 1..21: note = siRNA constructs for CLEC16A
  source 1..21: mol_type = other DNA
caaaccttagtgttgatgttt

SEQ ID NO: 54 - DNA, 21 bp, synthetic construct
  misc_feature 1..21: note = siRNA constructs for CLEC16A
  source 1..21: mol_type = other DNA
acttcattattccacaggctt

SEQ ID NO: 55 - DNA, 21 bp, synthetic construct
  misc_feature 1..21: note = siRNA constructs for CLEC16A
  source 1..21: mol_type = other DNA
tttccaggaaggtacttcatt

SEQ ID NO: 56 - DNA, 19 bp, synthetic construct
  misc_feature 1..19: note = siRNA constructs for CLEC16A
  source 1..19: mol_type = other DNA
gtatctcccttgtttttgt

SEQ ID NO: 57 - DNA, 21 bp, synthetic construct
  misc_feature 1..21: note = siRNA constructs for CLEC16A
  source 1..21: mol_type = other DNA
ggaagctgtctctgtttggtt

SEQ ID NO: 58 - DNA, 21 bp, synthetic construct
  misc_feature 1..21: note = siRNA constructs for CLEC16A
  source 1..21: mol_type = other DNA
tcttcacggtcccaactggtt

SEQ ID NO: 59 - DNA, 21 bp, synthetic construct
  misc_feature 1..21: note = siRNA constructs for CLEC16A
  source 1..21: mol_type = other DNA
ggccacagggtcgggagtctt

SEQ ID NO: 60 - DNA, 21 bp, synthetic construct
  misc_feature 1..21: note = siRNA constructs for CLEC16A
  source 1..21: mol_type = other DNA
gtctgatgcatctgggtcctt

SEQ ID NO: 61 - DNA, 21 bp, synthetic construct
  misc_feature 1..21: note = siRNA constructs for CLEC16A
  source 1..21: mol_type = other DNA
ccgagtagaaaggaacatctt

SEQ ID NO: 62 - DNA, 21 bp, synthetic construct
  misc_feature 1..21: note = siRNA constructs for CLEC16A
  source 1..21: mol_type = other DNA
tggcaggtctggaggaaagtt

SEQ ID NO: 63 - DNA, 21 bp, synthetic construct
  misc_feature 1..21: note = siRNA constructs for CLEC16A
  source 1..21: mol_type = other DNA
ggagacagagaccatgttctt

SEQ ID NO: 64 - DNA, 21 bp, synthetic construct
  misc_feature 1..21: note = siRNA constructs for CLEC16A
  source 1..21: mol_type = other DNA
cgaggagacagagaccatgtt

SEQ ID NO: 65 - DNA, 21 bp, synthetic construct
  misc_feature 1..21: note = siRNA constructs for CLEC16A
  source 1..21: mol_type = other DNA
aaactgccctcggcaggcctt

SEQ ID NO: 66 - DNA, 21 bp, synthetic construct
  misc_feature 1..21: note = siRNA constructs for CLEC16A
  source 1..21: mol_type = other DNA
ttggcaggactgtgtttcctt

SEQ ID NO: 67 - DNA, 19 bp, synthetic construct
  misc_feature 1..19: note = siRNA constructs for CLEC16A
  source 1..19: mol_type = other DNA
ctccttggcaggactgtgt

SEQ ID NO: 68 - DNA, 21 bp, synthetic construct
  misc_feature 1..21: note = siRNA constructs for CLEC16A
  source 1..21: mol_type = other DNA
tgggcgccactccccctcctt

SEQ ID NO: 69 - DNA, 19 bp, synthetic construct
  misc_feature 1..19: note = siRNA constructs for CLEC16A
  source 1..19: mol_type = other DNA
cctttctgagctgtgcgtt

SEQ ID NO: 70 - DNA, 21 bp, synthetic construct
  misc_feature 1..21: note = siRNA constructs for CLEC16A
  source 1..21: mol_type = other DNA
gtttgtccttcctttgggttt

SEQ ID NO: 71 - DNA, 21 bp, synthetic construct
  misc_feature 1..21: note = siRNA constructs for CLEC16A
  source 1..21: mol_type = other DNA
cttggggtggggcagccactt

SEQ ID NO: 72 - DNA, 21 bp, synthetic construct
  misc_feature 1..21: note = siRNA constructs for CLEC16A
  source 1..21: mol_type = other DNA
ctgggtgaggtggggtccttt

SEQ ID NO: 73 - DNA, 21 bp, synthetic construct
  misc_feature 1..21: note = siRNA constructs for CLEC16A
  source 1..21: mol_type = other DNA
acaaaaattatttacatattt

SEQ ID NO: 74 - DNA, 21 bp, synthetic construct
  misc_feature 1..21: note = siRNA constructs for CLEC16A
  source 1..21: mol_type = other DNA
tcactgggacaaaaattattt

SEQ ID NO: 75 - DNA, 21 bp, synthetic construct
  misc_feature 1..21: note = siRNA constructs for CLEC16A
  source 1..21: mol_type = other DNA
gttctcactgggacaaaaatt

SEQ ID NO: 76 - DNA, 21 bp, synthetic construct
  misc_feature 1..21: note = siRNA constructs for CLEC16A
  source 1..21: mol_type = other DNA
gctcagaggcatcgaggtttt

SEQ ID NO: 77 - DNA, 21 bp, synthetic construct
  misc_feature 1..21: note = siRNA constructs for CLEC16A
  source 1..21: mol_type = other DNA
aggctcagaggcatcgaggtt

SEQ ID NO: 78 - DNA, 20 bp, synthetic construct
  misc_feature 1..20: note = siRNA constructs for CLEC16A
  source 1..20: mol_type = other DNA
tcactcagtttaccccgatt

SEQ ID NO: 79 - DNA, 21 bp, synthetic construct
  misc_feature 1..21: note = siRNA constructs for CLEC16A
  source 1..21: mol_type = other DNA
ggaggaaccgcgagccgcctt

SEQ ID NO: 80 - DNA, 21 bp, synthetic construct
  misc_feature 1..21: note = siRNA constructs for CLEC16A
  source 1..21: mol_type = other DNA
gtggatgttgcgggaagtctt

SEQ ID NO: 81 - DNA, 21 bp, synthetic construct
  misc_feature 1..21: note = siRNA constructs for CLEC16A
  source 1..21: mol_type = other DNA
gtggtccaaggagtggatgtt

SEQ ID NO: 82 - DNA, 21 bp, synthetic construct
  misc_feature 1..21: note = siRNA constructs for CLEC16A
  source 1..21: mol_type = other DNA
tgttctgtgactgtggtgttt

SEQ ID NO: 83 - DNA, 21 bp, synthetic construct
  misc_feature 1..21: note = siRNA constructs for CLEC16A
  source 1..21: mol_type = other DNA
caaatacagagctgtcatttt

SEQ ID NO: 84 - DNA, 21 bp, synthetic construct
  misc_feature 1..21: note = siRNA constructs for CLEC16A
  source 1..21: mol_type = other DNA
gtcaaatacagagctgtcatt

SEQ ID NO: 85 - DNA, 21 bp, synthetic construct
  misc_feature 1..21: note = siRNA constructs for CLEC16A
  source 1..21: mol_type = other DNA
gaagaaaacaaacatattctt

SEQ ID NO: 86 - DNA, 21 bp, synthetic construct
  misc_feature 1..21: note = siRNA constructs for CLEC16A
  source 1..21: mol_type = other DNA
cgacttttgccgcaagatgtt

SEQ ID NO: 87 - DNA, 21 bp, synthetic construct
  misc_feature 1..21: note = siRNA constructs for CLEC16A
  source 1..21: mol_type = other DNA
gcacacgtaacggcccgactt

SEQ ID NO: 88 - DNA, 21 bp, synthetic construct
  misc_feature 1..21: note = siRNA constructs for CLEC16A
  source 1..21: mol_type = other DNA
gatgttctcaaagaggatgtt

SEQ ID NO: 89 - DNA, 21 bp, synthetic construct
  misc_feature 1..21: note = siRNA constructs for CLEC16A
  source 1..21: mol_type = other DNA
atagaatttacgtagttattt

SEQ ID NO: 90 - DNA, 19 bp, synthetic construct
  misc_feature 1..19: note = siRNA constructs for CLEC16A
  source 1..19: mol_type = other DNA
gatgatagaatttacgtag

SEQ ID NO: 91 - DNA, 21 bp, synthetic construct
  misc_feature 1..21: note = siRNA constructs for CLEC16A
  source 1..21: mol_type = other DNA
ttatgaacgatgatagaattt

SEQ ID NO: 92 - DNA, 19 bp, synthetic construct
  misc_feature 1..19: note = siRNA constructs for CLEC16A
  source 1..19: mol_type = other DNA
ttgagttttaacgaaagtg

SEQ ID NO: 93 - DNA, 21 bp, synthetic construct
  misc_feature 1..21: note = siRNA constructs for CLEC16A
  source 1..21: mol_type = other DNA
ggacagtgtggttgttgagtt

SEQ ID NO: 94 - DNA, 21 bp, synthetic construct
  misc_feature 1..21: note = siRNA constructs for CLEC16A
  source 1..21: mol_type = other DNA
gaaatggacagtgtggttgtt

SEQ ID NO: 95 - DNA, 21 bp, synthetic construct
  misc_feature 1..21: note = siRNA constructs for CLEC16A
  source 1..21: mol_type = other DNA
aaagtcattggtgtgctcatt

SEQ ID NO: 96 - DNA, 19 bp, synthetic construct
  misc_feature 1..19: note = siRNA constructs for CLEC16A
  source 1..19: mol_type = other DNA
aaccatgctttcagggtgg

SEQ ID NO: 97 - DNA, 21 bp, synthetic construct
  misc_feature 1..21: note = siRNA constructs for CLEC16A
  source 1..21: mol_type = other DNA
cagcaattctaaccatgcttt

SEQ ID NO: 98 - DNA, 21 bp, synthetic construct
  misc_feature 1..21: note = siRNA constructs for CLEC16A
  source 1..21: mol_type = other DNA
gttatggttcttacagcaatt

SEQ ID NO: 99 - DNA, 21 bp, synthetic construct
  misc_feature 1..21: note = siRNA constructs for CLEC16A
  source 1..21: mol_type = other DNA
acattcaaagttatggttctt

SEQ ID NO: 100 - DNA, 21 bp, synthetic construct
  misc_feature 1..21: note = siRNA constructs for CLEC16A
  source 1..21: mol_type = other DNA
tagacattcaaagttatggtt

SEQ ID NO: 101 - DNA, 21 bp, synthetic construct
  misc_feature 1..21: note = siRNA constructs for CLEC16A
  source 1..21: mol_type = other DNA
caatgacactttatagacatt

SEQ ID NO: 102 - DNA, 21 bp, synthetic construct
  misc_feature 1..21: note = siRNA constructs for CLEC16A
  source 1..21: mol_type = other DNA
ctggttatccaatgacacttt

SEQ ID NO: 103 - DNA, 19 bp, synthetic construct
  misc_feature 1..19: note = siRNA constructs for CLEC16A
  source 1..19: mol_type = other DNA
gtagtgcagcatggcctgg

SEQ ID NO: 104 - DNA, 21 bp, synthetic construct
  misc_feature 1..21: note = siRNA constructs for CLEC16A
  source 1..21: mol_type = other DNA
gaagtaaggaacagcagtttt

SEQ ID NO: 105 - DNA, 21 bp, synthetic construct
  misc_feature 1..21: note = siRNA constructs for CLEC16A
  source 1..21: mol_type = other DNA
gagaagtaaggaacagcagtt

SEQ ID NO: 106 - DNA, 21 bp, synthetic construct
  misc_feature 1..21: note = siRNA constructs for CLEC16A
  source 1..21: mol_type = other DNA
tgccaccagatcactcagttt

SEQ ID NO: 107 - DNA, 21 bp, synthetic construct
  misc_feature 1..21: note = siRNA constructs for CLEC16A
  source 1..21: mol_type = other DNA
gaggggcaggaagagcctgtt

SEQ ID NO: 108 - DNA, 21 bp, synthetic construct
  misc_feature 1..21: note = siRNA constructs for CLEC16A
  source 1..21: mol_type = other DNA
ttctcctcccttgtcctggtt

SEQ ID NO: 109 - DNA, 21 bp, synthetic construct
  misc_feature 1..21: note = siRNA constructs for CLEC16A
  source 1..21: mol_type = other DNA
tttcggccgttctcctccctt

SEQ ID NO: 110 - DNA, 19 bp, synthetic construct
  misc_feature 1..19: note = siRNA constructs for CLEC16A
  source 1..19: mol_type = other DNA
gcaggctaattttcggccg

SEQ ID NO: 111 - DNA, 19 bp, synthetic construct
  misc_feature 1..19: note = siRNA constructs for CLEC16A
  source 1..19: mol_type = other DNA
agacaccggcaggctaatt

SEQ ID NO: 112 - DNA, 21 bp, synthetic construct
  misc_feature 1..21: note = siRNA constructs for CLEC16A
  source 1..21: mol_type = other DNA
agagacaccggcaggctaatt

SEQ ID NO: 113 - DNA, 21 bp, synthetic construct
  misc_feature 1..21: note = siRNA constructs for CLEC16A
  source 1..21: mol_type = other DNA
atgctgggcttggcagaactt

SEQ ID NO: 114 - DNA, 21 bp, synthetic construct
  misc_feature 1..21: note = siRNA constructs for CLEC16A
  source 1..21: mol_type = other DNA
gaagcaccgaatgctgggctt

SEQ ID NO: 115 - DNA, 21 bp, synthetic construct
  misc_feature 1..21: note = siRNA constructs for CLEC16A
  source 1..21: mol_type = other DNA
ctcgagtgtctcggtgggttt

SEQ ID NO: 116 - DNA, 21 bp, synthetic construct
  misc_feature 1..21: note = siRNA constructs for CLEC16A
  source 1..21: mol_type = other DNA
ccgcctcttgcccttgtgctt

SEQ ID NO: 117 - DNA, 21 bp, synthetic construct
  misc_feature 1..21: note = siRNA constructs for CLEC16A
  source 1..21: mol_type = other DNA
ttgcacccgcctcttgccctt

SEQ ID NO: 118 - DNA, 21 bp, synthetic construct
  misc_feature 1..21: note = siRNA constructs for CLEC16A
  source 1..21: mol_type = other DNA
tctcttttgcacccgcctctt

SEQ ID NO: 119 - DNA, 21 bp, synthetic construct
  misc_feature 1..21: note = siRNA constructs for CLEC16A
  source 1..21: mol_type = other DNA
ttttgtagttgggtctctttt

SEQ ID NO: 120 - DNA, 21 bp, synthetic construct
  misc_feature 1..21: note = siRNA constructs for CLEC16A
  source 1..21: mol_type = other DNA
gtttttgtagttgggtctctt

SEQ ID NO: 121 - DNA, 21 bp, synthetic construct
  misc_feature 1..21: note = siRNA constructs for CLEC16A
  source 1..21: mol_type = other DNA
ttccccaacgtttttgtagtt

SEQ ID NO: 122 - DNA, 21 bp, synthetic construct
  misc_feature 1..21: note = siRNA constructs for CLEC16A
  source 1..21: mol_type = other DNA
ttcttcttccccaacgttttt

SEQ ID NO: 123 - DNA, 21 bp, synthetic construct
  misc_feature 1..21: note = siRNA constructs for CLEC16A
  source 1..21: mol_type = other DNA
tcttcttcttccccaacgttt

SEQ ID NO: 124 - DNA, 21 bp, synthetic construct
  misc_feature 1..21: note = siRNA constructs for CLEC16A
  source 1..21: mol_type = other DNA
ctttctcctcatcttcttctt

SEQ ID NO: 125 - DNA, 21 bp, synthetic construct
  misc_feature 1..21: note = siRNA constructs for CLEC16A
  source 1..21: mol_type = other DNA
gccctttctcctcatcttctt

SEQ ID NO: 126 - DNA, 21 bp, synthetic construct
  misc_feature 1..21: note = siRNA constructs for CLEC16A
  source 1..21: mol_type = other DNA
tagccttctcggcgtcttctt

SEQ ID NO: 127 - DNA, 21 bp, synthetic construct
  misc_feature 1..21: note = siRNA constructs for CLEC16A
  source 1..21: mol_type = other DNA
ctttagccttctcggcgtctt

SEQ ID NO: 128 - DNA, 21 bp, synthetic construct
  misc_feature 1..21: note = siRNA constructs for CLEC16A
  source 1..21: mol_type = other DNA
accctctgtacctttagcctt

SEQ ID NO: 129 - DNA, 21 bp, synthetic construct
  misc_feature 1..21: note = siRNA constructs for CLEC16A
  source 1..21: mol_type = other DNA
tgaaccaccctctgtaccttt

SEQ ID NO: 130 - DNA, 21 bp, synthetic construct
  misc_feature 1..21: note = siRNA constructs for CLEC16A
  source 1..21: mol_type = other DNA
ccactcgtcttgatgcctttt

SEQ ID NO: 131 - DNA, 21 bp, synthetic construct
  misc_feature 1..21: note = siRNA constructs for CLEC16A
  source 1..21: mol_type = other DNA
ccccactcgtcttgatgcctt

SEQ ID NO: 132 - DNA, 21 bp, synthetic construct
  misc_feature 1..21: note = siRNA constructs for CLEC16A
  source 1..21: mol_type = other DNA
ttcactctccccactcgtctt

SEQ ID NO: 133 - DNA, 21 bp, synthetic construct
  misc_feature 1..21: note = siRNA constructs for CLEC16A
  source 1..21: mol_type = other DNA
tgatcaccatctcgatctctt

SEQ ID NO: 134 - DNA, 21 bp, synthetic construct
  misc_feature 1..21: note = siRNA constructs for CLEC16A
  source 1..21: mol_type = other DNA
ggcggccagctctgagagctt

SEQ ID NO: 135 - DNA, 21 bp, synthetic construct
  misc_feature 1..21: note = siRNA constructs for CLEC16A
  source 1..21: mol_type = other DNA
tttctcctcgtccgtggtgtt

SEQ ID NO: 136 - DNA, 21 bp, synthetic construct
  misc_feature 1..21: note = siRNA constructs for CLEC16A
  source 1..21: mol_type = other DNA
gcaggtggcggcggcgctttt

SEQ ID NO: 137 - DNA, 21 bp, synthetic construct
  misc_feature 1..21: note = siRNA constructs for CLEC16A
  source 1..21: mol_type = other DNA
gagcaggtggcggcggcgctt

SEQ ID NO: 138 - DNA, 21 bp, synthetic construct
  misc_feature 1..21: note = siRNA constructs for CLEC16A
  source 1..21: mol_type = other DNA
ccaggaagggtctgctccatt

SEQ ID NO: 139 - DNA, 21 bp, synthetic construct
  misc_feature 1..21: note = siRNA constructs for CLEC16A
  source 1..21: mol_type = other DNA
ttcaggatccatgcctttatt

SEQ ID NO: 140 - DNA, 19 bp, synthetic construct
  misc_feature 1..19: note = siRNA constructs for CLEC16A
  source 1..19: mol_type = other DNA
tttttcaggatccatgcct

SEQ ID NO: 141 - DNA, 21 bp, synthetic construct
  misc_feature 1..21: note = siRNA constructs for CLEC16A
  source 1..21: mol_type = other DNA
gagctggattcgctctaattt

SEQ ID NO: 142 - DNA, 21 bp, synthetic construct
  misc_feature 1..21: note = siRNA constructs for CLEC16A
  source 1..21: mol_type = other DNA
tttggcacggggagctggatt

SEQ ID NO: 143 - DNA, 21 bp, synthetic construct
  misc_feature 1..21: note = siRNA constructs for CLEC16A
  source 1..21: mol_type = other DNA
gtggtcttctcggccgcattt

SEQ ID NO: 144 - DNA, 21 bp, synthetic construct
  misc_feature 1..21: note = siRNA constructs for CLEC16A
  source 1..21: mol_type = other DNA
cgggtggttgtaggtggtctt

SEQ ID NO: 145 - DNA, 21 bp, synthetic construct
  misc_feature 1..21: note = siRNA constructs for CLEC16A
  source 1..21: mol_type = other DNA
tctttcagctagcgggtggtt

SEQ ID NO: 146 - DNA, 21 bp, synthetic construct
  misc_feature 1..21: note = siRNA constructs for CLEC16A
  source 1..21: mol_type = other DNA
tcatgatcctgatgagtcttt

SEQ ID NO: 147 - DNA, 21 bp, synthetic construct
  misc_feature 1..21: note = siRNA constructs for CLEC16A
  source 1..21: mol_type = other DNA
gtacaaggtgaacactttctt

SEQ ID NO: 148 - DNA, 19 bp, synthetic construct
  misc_feature 1..19: note = siRNA constructs for CLEC16A
  source 1..19: mol_type = other DNA
gtcgtacaaggtgaacact

SEQ ID NO: 149 - DNA, 21 bp, synthetic construct
  misc_feature 1..21: note = siRNA constructs for CLEC16A
  source 1..21: mol_type = other DNA
tccacgttcatgggcttcatt

SEQ ID NO: 150 - DNA, 21 bp, synthetic construct
  misc_feature 1..21: note = siRNA constructs for CLEC16A
  source 1..21: mol_type = other DNA
atattccacgttcatgggctt

SEQ ID NO: 151 - DNA, 21 bp, synthetic construct
  misc_feature 1..21: note = siRNA constructs for CLEC16A
  source 1..21: mol_type = other DNA
catcatgagatattccacgtt

SEQ ID NO: 152 - DNA, 19 bp, synthetic construct
  misc_feature 1..19: note = siRNA constructs for CLEC16A
  source 1..19: mol_type = other DNA
aggcgtccatcatgagata

SEQ ID NO: 153 - DNA, 21 bp, synthetic construct
  misc_feature 1..21: note = siRNA constructs for CLEC16A
  source 1..21: mol_type = other DNA
cccgtcagtggcgtgcctgtt

SEQ ID NO: 154 - DNA, 21 bp, synthetic construct
  misc_feature 1..21: note = siRNA constructs for CLEC16A
  source 1..21: mol_type = other DNA
atcgccacacggcagccgctt

SEQ ID NO: 155 - DNA, 21 bp, synthetic construct
  misc_feature 1..21: note = siRNA constructs for CLEC16A
  source 1..21: mol_type = other DNA
ccggatggcccgccgggtctt

SEQ ID NO: 156 - DNA, 21 bp, synthetic construct
  misc_feature 1..21: note = siRNA constructs for CLEC16A
  source 1..21: mol_type = other DNA
tctcaggctcccctcgcaatt

SEQ ID NO: 157 - DNA, 21 bp, synthetic construct
  misc_feature 1..21: note = siRNA constructs for CLEC16A
  source 1..21: mol_type = other DNA
atccaggacatcatcagtctt

SEQ ID NO: 158 - DNA, 21 bp, synthetic construct
  misc_feature 1..21: note = siRNA constructs for CLEC16A
  source 1..21: mol_type = other DNA
tgcaatcaagtcgctgttatt

SEQ ID NO: 159 - DNA, 21 bp, synthetic construct
  misc_feature 1..21: note = siRNA constructs for CLEC16A
  source 1..21: mol_type = other DNA
acatgcaatcaagtcgctgtt

SEQ ID NO: 160 - DNA, 21 bp, synthetic construct
  misc_feature 1..21: note = siRNA constructs for CLEC16A
  source 1..21: mol_type = other DNA
ctggaccatgccgccatcctt

SEQ ID NO: 161 - DNA, 21 bp, synthetic construct
  misc_feature 1..21: note = siRNA constructs for CLEC16A
  source 1..21: mol_type = other DNA
ctgcaataggcctgcaaactt

SEQ ID NO: 162 - DNA, 21 bp, synthetic construct
  misc_feature 1..21: note = siRNA constructs for CLEC16A
  source 1..21: mol_type = other DNA
ctggaggatggggaagggctt

SEQ ID NO: 163 - DNA, 21 bp, synthetic construct
  misc_feature 1..21: note = siRNA constructs for CLEC16A
  source 1..21: mol_type = other DNA
gcctttggccaggcgctgctt

SEQ ID NO: 164 - DNA, 21 bp, synthetic construct
  misc_feature 1..21: note = siRNA constructs for CLEC16A
  source 1..21: mol_type = other DNA
ccttgcctggatgcggccttt

SEQ ID NO: 165 - DNA, 21 bp, synthetic construct
  misc_feature 1..21: note = siRNA constructs for CLEC16A
  source 1..21: mol_type = other DNA
ctctgcatcttcatgcgcctt

SEQ ID NO: 166 - DNA, 21 bp, synthetic construct
  misc_feature 1..21: note = siRNA constructs for CLEC16A
  source 1..21: mol_type = other DNA
ggcagctattctctgcatctt

SEQ ID NO: 167 - DNA, 21 bp, synthetic construct
  misc_feature 1..21: note = siRNA constructs for CLEC16A
  source 1..21: mol_type = other DNA
aggtccaggagggcagctatt

SEQ ID NO: 168 - DNA, 21 bp, synthetic construct
  misc_feature 1..21: note = siRNA constructs for CLEC16A
  source 1..21: mol_type = other DNA
acttcagtggtgggctggatt

SEQ ID NO: 169 - DNA, 21 bp, synthetic construct
  misc_feature 1..21: note = siRNA constructs for CLEC16A
  source 1..21: mol_type = other DNA
cgagtccaaaccccaggactt

SEQ ID NO: 170 - DNA, 21 bp, synthetic construct
  misc_feature 1..21: note = siRNA constructs for CLEC16A
  source 1..21: mol_type = other DNA
cacggcgaagcctggcacctt

SEQ ID NO: 171 - DNA, 19 bp, synthetic construct
  misc_feature 1..19: note = siRNA constructs for CLEC16A
  source 1..19: mol_type = other DNA
gacggggagctgtgctggt

SEQ ID NO: 172 - DNA, 21 bp, synthetic construct
  misc_feature 1..21: note = siRNA constructs for CLEC16A
  source 1..21: mol_type = other DNA
gaaggtgaggcttaggcagtt

SEQ ID NO: 173 - DNA, 21 bp, synthetic construct
  misc_feature 1..21: note = siRNA constructs for CLEC16A
  source 1..21: mol_type = other DNA
gcttccgtttcgttgacgatt

SEQ ID NO: 174 - DNA, 21 bp, synthetic construct
  misc_feature 1..21: note = siRNA constructs for CLEC16A
  source 1..21: mol_type = other DNA
agagtctgcttccgtttcgtt

SEQ ID NO: 175 - DNA, 21 bp, synthetic construct
  misc_feature 1..21: note = siRNA constructs for CLEC16A
  source 1..21: mol_type = other DNA
gcttagagtctgcttccgttt

SEQ ID NO: 176 - DNA, 21 bp, synthetic construct
  misc_feature 1..21: note = siRNA constructs for CLEC16A
  source 1..21: mol_type = other DNA
tgctgggcttagagtctgctt

SEQ ID NO: 177 - DNA, 21 bp, synthetic construct
  misc_feature 1..21: note = siRNA constructs for CLEC16A
  source 1..21: mol_type = other DNA
ggccacgttcttgctgggctt

SEQ ID NO: 178 - DNA, 21 bp, synthetic construct
  misc_feature 1..21: note = siRNA constructs for CLEC16A
  source 1..21: mol_type = other DNA
gggggacaagggtcagcgatt

SEQ ID NO: 179 - DNA, 21 bp, synthetic construct
  misc_feature 1..21: note = siRNA constructs for CLEC16A
  source 1..21: mol_type = other DNA
gctcagcacatgcagcctctt

SEQ ID NO: 180 - DNA, 21 bp, synthetic construct
  misc_feature 1..21: note = siRNA constructs for CLEC16A
  source 1..21: mol_type = other DNA
aacgtggggcttcttcccatt

SEQ ID NO: 181 - DNA, 21 bp, synthetic construct
  misc_feature 1..21: note = siRNA constructs for CLEC16A
  source 1..21: mol_type = other DNA
ttcaaggacaacgtggggctt

SEQ ID NO: 182 - DNA, 21 bp, synthetic construct
  misc_feature 1..21: note = siRNA constructs for CLEC16A
  source 1..21: mol_type = other DNA
atgcaaagtgaaaaaggaatt

SEQ ID NO: 183 - DNA, 21 bp, synthetic construct
  misc_feature 1..21: note = siRNA constructs for CLEC16A
  source 1..21: mol_type = other DNA
gccggtgcagtcatctgcatt

SEQ ID NO: 184 - DNA, 21 bp, synthetic construct
  misc_feature 1..21: note = siRNA constructs for CLEC16A
  source 1..21: mol_type = other DNA
cagtgacctggtccaattctt

SEQ ID NO: 185 - DNA, 21 bp, synthetic construct
  misc_feature 1..21: note = siRNA constructs for CLEC16A
  source 1..21: mol_type = other DNA
agtctgcttttctacaaattt

SEQ ID NO: 186 - DNA, 21 bp, synthetic construct
  misc_feature 1..21: note = siRNA constructs for CLEC16A
  source 1..21: mol_type = other DNA
tgtttatctaagtctgctttt

SEQ ID NO: 187 - DNA, 21 bp, synthetic construct
  misc_feature 1..21: note = siRNA constructs for CLEC16A
  source 1..21: mol_type = other DNA
gatgtttatctaagtctgctt

SEQ ID NO: 188 - DNA, 21 bp, synthetic construct
  misc_feature 1..21: note = siRNA constructs for CLEC16A
  source 1..21: mol_type = other DNA
gaagttttaaaaataaatgtt

SEQ ID NO: 189 - DNA, 21 bp, synthetic construct
  misc_feature 1..21: note = siRNA constructs for CLEC16A
  source 1..21: mol_type = other DNA
cttcttttaaatagaagtttt

SEQ ID NO: 190 - DNA, 21 bp, synthetic construct
  misc_feature 1..21: note = siRNA constructs for CLEC16A
  source 1..21: mol_type = other DNA
gacttcttttaaatagaagtt

SEQ ID NO: 191 - DNA, 21 bp, synthetic construct
  misc_feature 1..21: note = siRNA constructs for CLEC16A
  source 1..21: mol_type = other DNA
atgacatcaaaccttagtgtt

SEQ ID NO: 192 - DNA, 21 bp, synthetic construct
  misc_feature 1..21: note = siRNA constructs for CLEC16A
  source 1..21: mol_type = other DNA
tttcacatgacatcaaacctt

SEQ ID NO: 193 - DNA, 21 bp, synthetic construct
  misc_feature 1..21: note = siRNA constructs for CLEC16A
  source 1..21: mol_type = other DNA
aactgttattattacactttt

SEQ ID NO: 194 - DNA, 21 bp, synthetic construct
  misc_feature 1..21: note = siRNA constructs for CLEC16A
  source 1..21: mol_type = other DNA
ttaactgttattattacactt

SEQ ID NO: 195 - DNA, 21 bp, synthetic construct
  misc_feature 1..21: note = siRNA constructs for CLEC16A
  source 1..21: mol_type = other DNA
gaaatcttaactgttattatt

SEQ ID NO: 196 - DNA, 21 bp, synthetic construct
  misc_feature 1..21: note = siRNA constructs for CLEC16A
  source 1..21: mol_type = other DNA
catgaaatcttaactgttatt

SEQ ID NO: 197 - DNA, 21 bp, synthetic construct
  misc_feature 1..21: note = siRNA constructs for CLEC16A
  source 1..21: mol_type = other DNA
gatcatgaaatcttaactgtt

SEQ ID NO: 198 - DNA, 19 bp, synthetic construct
  misc_feature 1..19: note = siRNA constructs for CLEC16A
  source 1..19: mol_type = other DNA
tgaaaatgatcatgaaatc

SEQ ID NO: 199 - DNA, 21 bp, synthetic construct
  misc_feature 1..21: note = siRNA constructs for CLEC16A
  source 1..21: mol_type = other DNA
gtgagtaacaaagaatcactt

SEQ ID NO: 200 - DNA, 21 bp, synthetic construct
  misc_feature 1..21: note = siRNA constructs for CLEC16A
  source 1..21: mol_type = other DNA
attccacaggcttgcagagtt

SEQ ID NO: 201 - DNA, 21 bp, synthetic construct
  misc_feature 1..21: note = siRNA constructs for CLEC16A
  source 1..21: mol_type = other DNA
ccaggaaggtacttcattatt

SEQ ID NO: 202 - DNA, 21 bp, synthetic construct
  misc_feature 1..21: note = siRNA constructs for CLEC16A
  source 1..21: mol_type = other DNA
aaactttccaggaaggtactt

SEQ ID NO: 203 - DNA, 21 bp, synthetic construct
  misc_feature 1..21: note = siRNA constructs for CLEC16A
  source 1..21: mol_type = other DNA
ttaaaaaataatccaaacttt

SEQ ID NO: 204 - DNA, 21 bp, synthetic construct
  misc_feature 1..21: note = siRNA constructs for CLEC16A
  source 1..21: mol_type = other DNA
acatgtatctcccttgttttt

SEQ ID NO: 205 - DNA, 21 bp, synthetic construct
  misc_feature 1..21: note = siRNA constructs for CLEC16A
  source 1..21: mol_type = other DNA
atacatgtatctcccttgttt

SEQ ID NO: 206 - DNA, 19 bp, synthetic construct
  misc_feature 1..19: note = siRNA constructs for CLEC16A
  source 1..19: mol_type = other DNA
gagaatacatgtatctccc

SEQ ID NO: 207 - DNA, 21 bp, synthetic construct
  misc_feature 1..21: note = siRNA constructs for CLEC16A
  source 1..21: mol_type = other DNA
ccttttcctggagaaatcttt

SEQ ID NO: 208 - DNA, 19 bp, synthetic construct
  misc_feature 1..19: note = siRNA constructs for CLEC16A
  source 1..19: mol_type = other DNA
gctggctacattcctcctt

SEQ ID NO: 209 - DNA, 21 bp, synthetic construct
  misc_feature 1..21: note = siRNA constructs for CLEC16A
  source 1..21: mol_type = other DNA
gagctggctacattcctcctt

SEQ ID NO: 210 - DNA, 21 bp, synthetic construct
  misc_feature 1..21: note = siRNA constructs for CLEC16A
  source 1..21: mol_type = other DNA
gagtggggagctggctacatt

SEQ ID NO: 211 - DNA, 21 bp, synthetic construct
  misc_feature 1..21: note = siRNA constructs for CLEC16A
  source 1..21: mol_type = other DNA
aagctgtctctgtttggtttt

SEQ ID NO: 212 - DNA, 19 bp, synthetic construct
  misc_feature 1..19: note = siRNA constructs for CLEC16A
  source 1..19: mol_type = other DNA
tgctggaagctgtctctgt

SEQ ID NO: 213 - DNA, 21 bp, synthetic construct
  misc_feature 1..21: note = siRNA constructs for CLEC16A
  source 1..21: mol_type = other DNA
ggtcccaactggttccttctt

SEQ ID NO: 214 - DNA, 21 bp, synthetic construct
  misc_feature 1..21: note = siRNA constructs for CLEC16A
  source 1..21: mol_type = other DNA
cacggtcccaactggttcctt

SEQ ID NO: 215 - DNA, 21 bp, synthetic construct
  misc_feature 1..21: note = siRNA constructs for CLEC16A
  source 1..21: mol_type = other DNA
gagggtgtcttcctttgattt

SEQ ID NO: 216 - DNA, 21 bp, synthetic construct
  misc_feature 1..21: note = siRNA constructs for CLEC16A
  source 1..21: mol_type = other DNA
acgtagagggtgtcttccttt

SEQ ID NO: 217 - DNA, 21 bp, synthetic construct
  misc_feature 1..21: note = siRNA constructs for CLEC16A
  source 1..21: mol_type = other DNA
aggtgacgtagagggtgtctt

SEQ ID NO: 218 - DNA, 21 bp, synthetic construct
  misc_feature 1..21: note = siRNA constructs for CLEC16A
  source 1..21: mol_type = other DNA
ctctggggacaccatgccctt

SEQ ID NO: 219 - DNA, 21 bp, synthetic construct
  misc_feature 1..21: note = siRNA constructs for CLEC16A
  source 1..21: mol_type = other DNA
gaaccatggggtttccatatt

SEQ ID NO: 220 - DNA, 21 bp, synthetic construct
  misc_feature 1..21: note = siRNA constructs for CLEC16A
  source 1..21: mol_type = other DNA
gatgcatctgggtccttgctt

SEQ ID NO: 221 - DNA, 21 bp, synthetic construct
  misc_feature 1..21: note = siRNA constructs for CLEC16A
  source 1..21: mol_type = other DNA
ctgtgtttccttcatgaggtt

SEQ ID NO: 222 - DNA, 19 bp, synthetic construct
  misc_feature 1..19: note = siRNA constructs for CLEC16A
  source 1..19: mol_type = other DNA
gcacagctacccagagggc

SEQ ID NO: 223 - DNA, 19 bp, synthetic construct
  misc_feature 1..19: note = siRNA constructs for CLEC16A
  source 1..19: mol_type = other DNA
tctatggaagcaaaggtcc

SEQ ID NO: 224 - DNA, 21 bp, synthetic construct
  misc_feature 1..21: note = siRNA constructs for CLEC16A
  source 1..21: mol_type = other DNA
cccctttctgagctgtgcgtt

SEQ ID NO: 225 - DNA, 21 bp, synthetic construct
  misc_feature 1..21: note = siRNA constructs for CLEC16A
  source 1..21: mol_type = other DNA
tctgcccatgtggcccccttt

SEQ ID NO: 226 - DNA, 21 bp, synthetic construct
  misc_feature 1..21: note = siRNA constructs for CLEC16A
  source 1..21: mol_type = other DNA
gtcgtggtttgtccttccttt

SEQ ID NO: 227 - DNA, 19 bp, synthetic construct
  misc_feature 1..19: note = siRNA constructs for CLEC16A
  source 1..19: mol_type = other DNA
cggtggtcgtggtttgtcc

SEQ ID NO: 228 - DNA, 21 bp, synthetic construct
  misc_feature 1..21: note = siRNA constructs for CLEC16A
  source 1..21: mol_type = other DNA
tggccacggtggtcgtggttt

SEQ ID NO: 229 - DNA, 21 bp, synthetic construct
  misc_feature 1..21: note = siRNA constructs for CLEC16A
  source 1..21: mol_type = other DNA
cttccttctcttccagggatt

SEQ ID NO: 230 - DNA, 21 bp, synthetic construct
  misc_feature 1..21: note = siRNA constructs for CLEC16A
  source 1..21: mol_type = other DNA
ccaccctgccttccttctctt

SEQ ID NO: 231 - DNA, 21 bp, synthetic construct
  misc_feature 1..21: note = siRNA constructs for CLEC16A
  source 1..21: mol_type = other DNA
ccgctccaccctgccttcctt

SEQ ID NO: 232 - DNA, 21 bp, synthetic construct
  misc_feature 1..21: note = siRNA constructs for CLEC16A
  source 1..21: mol_type = other DNA
ccccccgctccaccctgcctt

SEQ ID NO: 233 - DNA, 21 bp, synthetic construct
  misc_feature 1..21: note = siRNA constructs for CLEC16A
  source 1..21: mol_type = other DNA
ccttctctccatgatggtctt

SEQ ID NO: 234 - DNA, 21 bp, synthetic construct
  misc_feature 1..21: note = siRNA constructs for CLEC16A
  source 1..21: mol_type = other DNA
tctcctgatgctgtggtcctt

SEQ ID NO: 235 - DNA, 21 bp, synthetic construct
  misc_feature 1..21: note = siRNA constructs for CLEC16A
  source 1..21: mol_type = other DNA
ccctcttgctcttaaaaaatt

SEQ ID NO: 236 - DNA, 21 bp, synthetic construct
  misc_feature 1..21: note = siRNA constructs for CLEC16A
  source 1..21: mol_type = other DNA
ctctctacccctcttgctctt

SEQ ID NO: 237 - DNA, 21 bp, synthetic construct
  misc_feature 1..21: note = siRNA constructs for CLEC16A
  source 1..21: mol_type = other DNA
ttgatcctctctacccctctt

SEQ ID NO: 238 - DNA, 19 bp, synthetic construct
  misc_feature 1..19: note = siRNA constructs for CLEC16A
  source 1..19: mol_type = other DNA
atctccagccagggccagc

SEQ ID NO: 239 - DNA, 21 bp, synthetic construct
  misc_feature 1..21: note = siRNA constructs for CLEC16A
  source 1..21: mol_type = other DNA
gccccacagacagagtcattt

SEQ ID NO: 240 - DNA, 21 bp, synthetic construct
  misc_feature 1..21: note = siRNA constructs for CLEC16A
  source 1..21: mol_type = other DNA
gccacttgccttctctagttt

SEQ ID NO: 241 - DNA, 21 bp, synthetic construct
  misc_feature 1..21: note = siRNA constructs for CLEC16A
  source 1..21: mol_type = other DNA
ggtggggcagccacttgcctt

SEQ ID NO: 242 - DNA, 21 bp, synthetic construct
  misc_feature 1..21: note = siRNA constructs for CLEC16A
  source 1..21: mol_type = other DNA
tgttcctcctggtcacgcctt

SEQ ID NO: 243 - DNA, 21 bp, synthetic construct
  misc_feature 1..21: note = siRNA constructs for CLEC16A
  source 1..21: mol_type = other DNA
tggagtgagctgcaggctgtt

SEQ ID NO: 244 - DNA, 21 bp, synthetic construct
  misc_feature 1..21: note = siRNA constructs for CLEC16A
  source 1..21: mol_type = other DNA
ccagttggagcccagagactt

SEQ ID NO: 245 - DNA, 21 bp, synthetic construct
  misc_feature 1..21: note = siRNA constructs for CLEC16A
  source 1..21: mol_type = other DNA
tcagtggttacaagaccagtt

SEQ ID NO: 246 - DNA, 21 bp, synthetic construct
  misc_feature 1..21: note = siRNA constructs for CLEC16A
  source 1..21: mol_type = other DNA
ctccttcagtgctcagtggtt

SEQ ID NO: 247 - DNA, 21 bp, synthetic construct
  misc_feature 1..21: note = siRNA constructs for CLEC16A
  source 1..21: mol_type = other DNA
ctgaccaagacctctctcctt

SEQ ID NO: 248 - DNA, 21 bp, synthetic construct
  misc_feature 1..21: note = siRNA constructs for CLEC16A
  source 1..21: mol_type = other DNA
ctggtcctcccagtcaccttt

SEQ ID NO: 249 - DNA, 21 bp, synthetic construct
  misc_feature 1..21: note = siRNA constructs for CLEC16A
  source 1..21: mol_type = other DNA
ctgccctgagcagtgtctttt

SEQ ID NO: 250 - DNA, 21 bp, synthetic construct
  misc_feature 1..21: note = siRNA constructs for CLEC16A
  source 1..21: mol_type = other DNA
ccctgccctgagcagtgtctt

SEQ ID NO: 251 - DNA, 21 bp, synthetic construct
  misc_feature 1..21: note = siRNA constructs for CLEC16A
  source 1..21: mol_type = other DNA
catggggactgcccttttctt

SEQ ID NO: 252 - DNA, 21 bp, synthetic construct
  misc_feature 1..21: note = siRNA constructs for CLEC16A
  source 1..21: mol_type = other DNA
ccacatggggactgccctttt

SEQ ID NO: 253 - DNA, 19 bp, synthetic construct
  misc_feature 1..19: note = siRNA constructs for CLEC16A
  source 1..19: mol_type = other DNA
gcccacatggggactgccc

SEQ ID NO: 254 - DNA, 21 bp, synthetic construct
  misc_feature 1..21: note = siRNA constructs for CLEC16A
  source 1..21: mol_type = other DNA
ggtgtcccccagacgcaagtt

SEQ ID NO: 255 - DNA, 21 bp, synthetic construct
  misc_feature 1..21: note = siRNA constructs for CLEC16A
  source 1..21: mol_type = other DNA
ctccttacataagcaaagctt

SEQ ID NO: 256 - DNA, 21 bp, synthetic construct
  misc_feature 1..21: note = siRNA constructs for CLEC16A
  source 1..21: mol_type = other DNA
ggctggctcccagacctcctt

SEQ ID NO: 257 - DNA, 21 bp, synthetic construct
  misc_feature 1..21: note = siRNA constructs for CLEC16A
  source 1..21: mol_type = other DNA
agcgccccagctatgaggttt

SEQ ID NO: 258 - DNA, 21 bp, synthetic construct
  misc_feature 1..21: note = siRNA constructs for CLEC16A
  source 1..21: mol_type = other DNA
cttcccacactcctggctctt

SEQ ID NO: 259 - DNA, 21 bp, synthetic construct
  misc_feature 1..21: note = siRNA constructs for CLEC16A
  source 1..21: mol_type = other DNA
acagcccccactgtgggcctt

SEQ ID NO: 260 - DNA, 21 bp, synthetic construct
  misc_feature 1..21: note = siRNA constructs for CLEC16A
  source 1..21: mol_type = other DNA
aagtgctctctgcagggactt

SEQ ID NO: 261 - DNA, 21 bp, synthetic construct
  misc_feature 1..21: note = siRNA constructs for CLEC16A
  source 1..21: mol_type = other DNA
gccagccctgctccctgactt

SEQ ID NO: 262 - DNA, 21 bp, synthetic construct
  misc_feature 1..21: note = siRNA constructs for CLEC16A
  source 1..21: mol_type = other DNA
caaagccaaggtttgggagtt

SEQ ID NO: 263 - DNA, 21 bp, synthetic construct
  misc_feature 1..21: note = siRNA constructs for CLEC16A
  source 1..21: mol_type = other DNA
caatattcaaagccaaggttt

SEQ ID NO: 264 - DNA, 19 bp, synthetic construct
  misc_feature 1..19: note = siRNA constructs for CLEC16A
  source 1..19: mol_type = other DNA
gcacacctccacaacaata

SEQ ID NO: 265 - DNA, 21 bp, synthetic construct
  misc_feature 1..21: note = siRNA constructs for CLEC16A
  source 1..21: mol_type = other DNA
acctgctgggacaggtacctt

SEQ ID NO: 266 - DNA, 21 bp, synthetic construct
  misc_feature 1..21: note = siRNA constructs for CLEC16A
  source 1..21: mol_type = other DNA
cgatggtgaaggctggccctt

SEQ ID NO: 267 - DNA, 19 bp, synthetic construct
  misc_feature 1..19: note = siRNA constructs for CLEC16A
  source 1..19: mol_type = other DNA
gtgaacgcaagtgtctggg

SEQ ID NO: 268 - DNA, 21 bp, synthetic construct
  misc_feature 1..21: note = siRNA constructs for CLEC16A
  source 1..21: mol_type = other DNA
cacctgccccttaggttgctt

SEQ ID NO: 269 - DNA, 21 bp, synthetic construct
  misc_feature 1..21: note = siRNA constructs for CLEC16A
  source 1..21: mol_type = other DNA
tcttcacctgccccttaggtt

SEQ ID NO: 270 - DNA, 21 bp, synthetic construct
  misc_feature 1..21: note = siRNA constructs for CLEC16A
  source 1..21: mol_type = other DNA
gcgtctggcagggctgcgctt

SEQ ID NO: 271 - DNA, 21 bp, synthetic construct
  misc_feature 1..21: note = siRNA constructs for CLEC16A
  source 1..21: mol_type = other DNA
acggtgcatctcagagacctt

SEQ ID NO: 272 - DNA, 21 bp, synthetic construct
  misc_feature 1..21: note = siRNA constructs for CLEC16A
  source 1..21: mol_type = other DNA
atcagttcaccccacgccttt

SEQ ID NO: 273 - DNA, 21 bp, synthetic construct
  misc_feature 1..21: note = siRNA constructs for CLEC16A
  source 1..21: mol_type = other DNA
acaagaagatcaaaatcagtt

SEQ ID NO: 274 - DNA, 21 bp, synthetic construct
  misc_feature 1..21: note = siRNA constructs for CLEC16A
  source 1..21: mol_type = other DNA
aatgcttcagatttatttatt

SEQ ID NO: 275 - DNA, 21 bp, synthetic construct
  misc_feature 1..21: note = siRNA constructs for CLEC16A
  source 1..21: mol_type = other DNA
ttaaatgcttcagatttattt

SEQ ID NO: 276 - DNA, 21 bp, synthetic construct
  misc_feature 1..21: note = siRNA constructs for CLEC16A
  source 1..21: mol_type = other DNA
tacattaaatgcttcagattt

SEQ ID NO: 277 - DNA, 21 bp, synthetic construct
  misc_feature 1..21: note = siRNA constructs for CLEC16A
  source 1..21: mol_type = other DNA
agatgactacattaaatgctt

SEQ ID NO: 278 - DNA, 21 bp, synthetic construct
  misc_feature 1..21: note = siRNA constructs for CLEC16A
  source 1..21: mol_type = other DNA
caatgtcaagatgactacatt

SEQ ID NO: 279 - DNA, 21 bp, synthetic construct
  misc_feature 1..21: note = siRNA constructs for CLEC16A
  source 1..21: mol_type = other DNA
aaaaattatttacatattttt

SEQ ID NO: 280 - DNA, 21 bp, synthetic construct
  misc_feature 1..21: note = siRNA constructs for CLEC16A
  source 1..21: mol_type = other DNA
tgctggcgaaagcaggtactt

SEQ ID NO: 281 - DNA, 21 bp, synthetic construct
  misc_feature 1..21: note = siRNA constructs for CLEC16A
  source 1..21: mol_type = other DNA
tttcatttccaccctcgtgtt

SEQ ID NO: 282 - DNA, 21 bp, synthetic construct
  misc_feature 1..21: note = siRNA constructs for CLEC16A
  source 1..21: mol_type = other DNA
aggaagttccagttttcattt

SEQ ID NO: 283 - DNA, 21 bp, synthetic construct
  misc_feature 1..21: note = siRNA constructs for CLEC16A
  source 1..21: mol_type = other DNA
ttacaaggaagttccagtttt

SEQ ID NO: 284 - DNA, 21 bp, synthetic construct
  misc_feature 1..21: note = siRNA constructs for CLEC16A
  source 1..21: mol_type = other DNA
atttacaaggaagttccagtt

SEQ ID NO: 285 - DNA, 21 bp, synthetic construct
  misc_feature 1..21: note = siRNA constructs for CLEC16A
  source 1..21: mol_type = other DNA
gtttaaatttacaaggaagtt

SEQ ID NO: 286 - DNA, 21 bp, synthetic construct
  misc_feature 1..21: note = siRNA constructs for CLEC16A
  source 1..21: mol_type = other DNA
atttaaacttggcaataaatt

SEQ ID NO: 287 - DNA, 21 bp, synthetic construct
  misc_feature 1..21: note = siRNA constructs for CLEC16A
  source 1..21: mol_type = other DNA
acttggcaataaaagagaatt

SEQ ID NO: 288 - DNA, 21 bp, synthetic construct
  misc_feature 1..21: note = siRNA constructs for CLEC16A
  source 1..21: mol_type = other DNA
cttctatttaaaagaagtctt

SEQ ID NO: 289 - DNA, 21 bp, synthetic construct
  misc_feature 1..21: note = siRNA constructs for CLEC16A
  source 1..21: mol_type = other DNA
acaccacagtcacagaacatt

SEQ ID NO: 290 - DNA, 21 bp, synthetic construct
  misc_feature 1..21: note = siRNA constructs for CLEC16A
  source 1..21: mol_type = other DNA
tatgtttgttttcttcttgtt

SEQ ID NO: 291 - DNA, 21 bp, synthetic construct
  misc_feature 1..21: note = siRNA constructs for CLEC16A
  source 1..21: mol_type = other DNA
catcctctttgagaacatctt

SEQ ID NO: 292 - DNA, 19 bp, synthetic construct
  misc_feature 1..19: note = siRNA constructs for CLEC16A
  source 1..19: mol_type = other DNA
attctatcatcgttcataa

SEQ ID NO: 293 - DNA, 19 bp, synthetic construct
  misc_feature 1..19: note = siRNA constructs for CLEC16A
  source 1..19: mol_type = other DNA
cactttcgttaaaactcaa

SEQ ID NO: 294 - DNA, 21 bp, synthetic construct
  misc_feature 1..21: note = siRNA constructs for CLEC16A
  source 1..21: mol_type = other DNA
aactcaacaaccacactgttt

SEQ ID NO: 295 - DNA, 21 bp, synthetic construct
  misc_feature 1..21: note = siRNA constructs for CLEC16A
  source 1..21: mol_type = other DNA
ctcaacaaccacactgtcctt

SEQ ID NO: 296 - DNA, 21 bp, synthetic construct
  misc_feature 1..21: note = siRNA constructs for CLEC16A
  source 1..21: mol_type = other DNA
agcatggttagaattgctgtt

SEQ ID NO: 297 - DNA, 21 bp, synthetic construct
  misc_feature 1..21: note = siRNA constructs for CLEC16A
  source 1..21: mol_type = other DNA
ccataactttgaatgtctatt

SEQ ID NO: 298 - DNA, 21 bp, synthetic construct
  misc_feature 1..21: note = siRNA constructs for CLEC16A
  source 1..21: mol_type = other DNA
aactgctgttccttacttctt

SEQ ID NO: 299 - DNA, 21 bp, synthetic construct
  misc_feature 1..21: note = siRNA constructs for CLEC16A
  source 1..21: mol_type = other DNA
ccaggacaagggaggagaatt

SEQ ID NO: 300 - DNA, 19 bp, synthetic construct
  misc_feature 1..19: note = siRNA constructs for CLEC16A
  source 1..19: mol_type = other DNA
ttagcctgccggtgtctct

SEQ ID NO: 301 - DNA, 21 bp, synthetic construct
  misc_feature 1..21: note = siRNA constructs for CLEC16A
  source 1..21: mol_type = other DNA
gaggcgggtgcaaaagagatt

SEQ ID NO: 302 - DNA, 21 bp, synthetic construct
  misc_feature 1..21: note = siRNA constructs for CLEC16A
  source 1..21: mol_type = other DNA
aagagacccaactacaaaatt

SEQ ID NO: 303 - DNA, 21 bp, synthetic construct
  misc_feature 1..21: note = siRNA constructs for CLEC16A
  source 1..21: mol_type = other DNA
gaagaagatgaggagaaagtt

SEQ ID NO: 304 - DNA, 21 bp, synthetic construct
  misc_feature 1..21: note = siRNA constructs for CLEC16A
  source 1..21: mol_type = other DNA
gaagatgaggagaaagggctt

SEQ ID NO: 305 - DNA, 21 bp, synthetic construct
  misc_feature 1..21: note = siRNA constructs for CLEC16A
  source 1..21: mol_type = other DNA
ggctaaaggtacagagggttt

SEQ ID NO: 306 - DNA, 21 bp, synthetic construct
  misc_feature 1..21: note = siRNA constructs for CLEC16A
  source 1..21: mol_type = other DNA
aggtacagagggtggttcatt

SEQ ID NO: 307 - DNA, 21 bp, synthetic construct
  misc_feature 1..21: note = siRNA constructs for CLEC16A
  source 1..21: mol_type = other DNA
gagatcgagatggtgatcatt

SEQ ID NO: 308 - DNA, 21 bp, synthetic construct
  misc_feature 1..21: note = siRNA constructs for CLEC16A
  source 1..21: mol_type = other DNA
tggagcagacccttcctggtt

SEQ ID NO: 309 - DNA, 21 bp, synthetic construct
  misc_feature 1..21: note = siRNA constructs for CLEC16A
  source 1..21: mol_type = other DNA
agactcatcaggatcatgatt

SEQ ID NO: 310 - DNA, 21 bp, synthetic construct
  misc_feature 1..21: note = siRNA constructs for CLEC16A
  source 1..21: mol_type = other DNA
caacgctgcccagccagattt

SEQ ID NO: 311 - DNA, 21 bp, synthetic construct
  misc_feature 1..21: note = siRNA constructs for CLEC16A
  source 1..21: mol_type = other DNA
tgaagcccatgaacgtggatt

SEQ ID NO: 312 - DNA, 21 bp, synthetic construct
  misc_feature 1..21: note = siRNA constructs for CLEC16A
  source 1..21: mol_type = other DNA
cgtggaatatctcatgatgtt

SEQ ID NO: 313 - DNA, 21 bp, synthetic construct
  misc_feature 1..21: note = siRNA constructs for CLEC16A
  source 1..21: mol_type = other DNA
caggcacgccactgacgggtt

SEQ ID NO: 314 - DNA, 21 bp, synthetic construct
  misc_feature 1..21: note = siRNA constructs for CLEC16A
  source 1..21: mol_type = other DNA
gcggctgccgtgtggcgattt

SEQ ID NO: 315 - DNA, 21 bp, synthetic construct
  misc_feature 1..21: note = siRNA constructs for CLEC16A
  source 1..21: mol_type = other DNA
gacccggcgggccatccggtt

SEQ ID NO: 316 - DNA, 21 bp, synthetic construct
  misc_feature 1..21: note = siRNA constructs for CLEC16A
  source 1..21: mol_type = other DNA
ttgcgaggggagcctgagatt

SEQ ID NO: 317 - DNA, 21 bp, synthetic construct
  misc_feature 1..21: note = siRNA constructs for CLEC16A
  source 1..21: mol_type = other DNA
tcgtcaacgaaacggaagctt

SEQ ID NO: 318 - DNA, 21 bp, synthetic construct
  misc_feature 1..21: note = siRNA constructs for CLEC16A
  source 1..21: mol_type = other DNA
cgaaacggaagcagactcttt

SEQ ID NO: 319 - DNA, 21 bp, synthetic construct
  misc_feature 1..21: note = siRNA constructs for CLEC16A
  source 1..21: mol_type = other DNA
acggaagcagactctaagctt

SEQ ID NO: 320 - DNA, 19 bp, synthetic construct
  misc_feature 1..19: note = siRNA constructs for CLEC16A
  source 1..19: mol_type = other DNA
cgtggccaggagcgcagcc

SEQ ID NO: 321 - DNA, 21 bp, synthetic construct
  misc_feature 1..21: note = siRNA constructs for CLEC16A
  source 1..21: mol_type = other DNA
gaggctgcatgtgctgagctt

SEQ ID NO: 322 - DNA, 21 bp, synthetic construct
  misc_feature 1..21: note = siRNA constructs for CLEC16A
  source 1..21: mol_type = other DNA
gccccacgttgtccttgaatt

SEQ ID NO: 323 - DNA, 21 bp, synthetic construct
  misc_feature 1..21: note = siRNA constructs for CLEC16A
  source 1..21: mol_type = other DNA
gcagacttagataaacatctt

SEQ ID NO: 324 - DNA, 21 bp, synthetic construct
  misc_feature 1..21: note = siRNA constructs for CLEC16A
  source 1..21: mol_type = other DNA
acatctcctttggatattttt

SEQ ID NO: 325 - DNA, 19 bp, synthetic construct
  misc_feature 1..19: note = siRNA constructs for CLEC16A
  source 1..19: mol_type = other DNA
catttatttttaaaacttc

SEQ ID NO: 326 - DNA, 21 bp, synthetic construct
  misc_feature 1..21: note = siRNA constructs for CLEC16A
  source 1..21: mol_type = other DNA
aacttctatttaaaagaagtt

SEQ ID NO: 327 - DNA, 21 bp, synthetic construct
  misc_feature 1..21: note = siRNA constructs for CLEC16A
  source 1..21: mol_type = other DNA
gtccaaaaacatcaacacttt

SEQ ID NO: 328 - DNA, 21 bp, synthetic construct
  misc_feature 1..21: note = siRNA constructs for CLEC16A
  source 1..21: mol_type = other DNA
ggtttgatgtcatgtgaaatt

SEQ ID NO: 329 - DNA, 19 bp, synthetic construct
  misc_feature 1..19: note = siRNA constructs for CLEC16A
  source 1..19: mol_type = other DNA
aagtgtaataataacagtt

SEQ ID NO: 330 - DNA, 21 bp, synthetic construct
  misc_feature 1..21: note = siRNA constructs for CLEC16A
  source 1..21: mol_type = other DNA
gtgtaataataacagttaatt

SEQ ID NO: 331 - DNA, 21 bp, synthetic construct
  misc_feature 1..21: note = siRNA constructs for CLEC16A
  source 1..21: mol_type = other DNA
taataacagttaagatttctt

SEQ ID NO: 332 - DNA, 21 bp, synthetic construct
  misc_feature 1..21: note = siRNA constructs for CLEC16A
  source 1..21: mol_type = other DNA
taacagttaagatttcatgtt

SEQ ID NO: 333 - DNA, 21 bp, synthetic construct
  misc_feature 1..21: note = siRNA constructs for CLEC16A
  source 1..21: mol_type = other DNA
cagttaagatttcatgatctt

SEQ ID NO: 334 - DNA, 21 bp, synthetic construct
  misc_feature 1..21: note = siRNA constructs for CLEC16A
  source 1..21: mol_type = other DNA
gatttcatgatcattttcatt

SEQ ID NO: 335 - DNA, 21 bp, synthetic construct
  misc_feature 1..21: note = siRNA constructs for CLEC16A
  source 1..21: mol_type = other DNA
gtaccttcctggaaagttttt

SEQ ID NO: 336 - DNA, 19 bp, synthetic construct
  misc_feature 1..19: note = siRNA constructs for CLEC16A
  source 1..19: mol_type = other DNA
agatttctccaggaaaagg

SEQ ID NO: 337 - DNA, 21 bp, synthetic construct
  misc_feature 1..21: note = siRNA constructs for CLEC16A
  source 1..21: mol_type = other DNA
aaggaggaatgtagccagctt

SEQ ID NO: 338 - DNA, 21 bp, synthetic construct
  misc_feature 1..21: note = siRNA constructs for CLEC16A
  source 1..21: mol_type = other DNA
ggaggaatgtagccagctctt

SEQ ID NO: 339 - DNA, 21 bp, synthetic construct
  misc_feature 1..21: note = siRNA constructs for CLEC16A
  source 1..21: mol_type = other DNA
tgtagccagctccccactctt

SEQ ID NO: 340 - DNA, 21 bp, synthetic construct
  misc_feature 1..21: note = siRNA constructs for CLEC16A
  source 1..21: mol_type = other DNA
aaccaaacagagacagctttt

SEQ ID NO: 341 - DNA, 20 bp, synthetic construct
  misc_feature 1..20: note = siRNA constructs for CLEC16A
  source 1..20: mol_type = other DNA
ccaaacgagacagcttcctt

SEQ ID NO: 342 - DNA, 21 bp, synthetic construct
  misc_feature 1..21: note = siRNA constructs for CLEC16A
  source 1..21: mol_type = other DNA
acagagacagcttccagcatt

SEQ ID NO: 343 - DNA, 21 bp, synthetic construct
  misc_feature 1..21: note = siRNA constructs for CLEC16A
  source 1..21: mol_type = other DNA
atcaaaggaagacaccctctt

SEQ ID NO: 344 - DNA, 21 bp, synthetic construct
  misc_feature 1..21: note = siRNA constructs for CLEC16A
  source 1..21: mol_type = other DNA
cgcacagctcagaaaggggtt

SEQ ID NO: 345 - DNA, 21 bp, synthetic construct
  misc_feature 1..21: note = siRNA constructs for CLEC16A
  source 1..21: mol_type = other DNA
agggggccacatgggcagatt

SEQ ID NO: 346 - DNA, 21 bp, synthetic construct
  misc_feature 1..21: note = siRNA constructs for CLEC16A
  source 1..21: mol_type = other DNA
acccaaaggaaggacaaactt

SEQ ID NO: 347 - DNA, 19 bp, synthetic construct
  misc_feature 1..19: note = siRNA constructs for CLEC16A
  source 1..19: mol_type = other DNA
aggaaggacaaaccacgac

SEQ ID NO: 348 - DNA, 19 bp, synthetic construct
  misc_feature 1..19: note = siRNA constructs for CLEC16A
  source 1..19: mol_type = other DNA
ggacaaaccacgaccaccg

SEQ ID NO: 349 - DNA, 21 bp, synthetic construct
  misc_feature 1..21: note = siRNA constructs for CLEC16A
  source 1..21: mol_type = other DNA
accacgaccaccgtggccatt

SEQ ID NO: 350 - DNA, 21 bp, synthetic construct
  misc_feature 1..21: note = siRNA constructs for CLEC16A
  source 1..21: mol_type = other DNA
actagagaaggcaagtggctt

SEQ ID NO: 351 - DNA, 21 bp, synthetic construct
  misc_feature 1..21: note = siRNA constructs for CLEC16A
  source 1..21: mol_type = other DNA
ggcgtgaccaggaggaacatt

SEQ ID NO: 352 - DNA, 21 bp, synthetic construct
  misc_feature 1..21: note = siRNA constructs for CLEC16A
  source 1..21: mol_type = other DNA
gctttgcttatgtaaggagtt

SEQ ID NO: 353 - DNA, 21 bp, synthetic construct
  misc_feature 1..21: note = siRNA constructs for CLEC16A
  source 1..21: mol_type = other DNA
gtcagggagcagggctggctt

SEQ ID NO: 354 - DNA, 21 bp, synthetic construct
  misc_feature 1..21: note = siRNA constructs for CLEC16A
  source 1..21: mol_type = other DNA
accttggctttgaatattgtt

SEQ ID NO: 355 - DNA, 19 bp, synthetic construct
  misc_feature 1..19: note = siRNA constructs for CLEC16A
  source 1..19: mol_type = other DNA
gggccagccttcaccatcg

SEQ ID NO: 356 - DNA, 19 bp, synthetic construct
  misc_feature 1..19: note = siRNA constructs for CLEC16A
  source 1..19: mol_type = other DNA
ataaatctgaagcatttaa

SEQ ID NO: 357 - DNA, 21 bp, synthetic construct
  misc_feature 1..21: note = siRNA constructs for CLEC16A
  source 1..21: mol_type = other DNA
ggcggctcgcggttcctcctt

SEQ ID NO: 358 - DNA, 21 bp, synthetic construct
  misc_feature 1..21: note = siRNA constructs for CLEC16A
  source 1..21: mol_type = other DNA
gacttcccgcaacatccactt

SEQ ID NO: 359 - DNA, 21 bp, synthetic construct
  misc_feature 1..21: note = siRNA constructs for CLEC16A
  source 1..21: mol_type = other DNA
catccactccttggaccactt

SEQ ID NO: 360 - DNA, 21 bp, synthetic construct
  misc_feature 1..21: note = siRNA constructs for CLEC16A
  source 1..21: mol_type = other DNA
gtatctgtaccacgttttgtt

SEQ ID NO: 361 - DNA, 21 bp, synthetic construct
  misc_feature 1..21: note = siRNA constructs for CLEC16A
  source 1..21: mol_type = other DNA
aaacaccacagtcacagaatt

SEQ ID NO: 362 - DNA, 21 bp, synthetic construct
  misc_feature 1..21: note = siRNA constructs for CLEC16A
  source 1..21: mol_type = other DNA
cagaaccggaacctgctagtt

SEQ ID NO: 363 - DNA, 21 bp, synthetic construct
  misc_feature 1..21: note = siRNA constructs for CLEC16A
  source 1..21: mol_type = other DNA
ccggaacctgctagtggagtt

SEQ ID NO: 364 - DNA, 21 bp, synthetic construct
  misc_feature 1..21: note = siRNA constructs for CLEC16A
  source 1..21: mol_type = other DNA
cctgctagtggagaccatctt

SEQ ID NO: 365 - DNA, 21 bp, synthetic construct
  misc_feature 1..21: note = siRNA constructs for CLEC16A
  source 1..21: mol_type = other DNA
aatgacagctctgtatttgtt

SEQ ID NO: 366 - DNA, 21 bp, synthetic construct
  misc_feature 1..21: note = siRNA constructs for CLEC16A
  source 1..21: mol_type = other DNA
tgacagctctgtatttgactt

SEQ ID NO: 367 - DNA, 21 bp, synthetic construct
  misc_feature 1..21: note = siRNA constructs for CLEC16A
  source 1..21: mol_type = other DNA
gaatatgtttgttttcttctt

SEQ ID NO: 368 - DNA, 21 bp, synthetic construct
  misc_feature 1..21: note = siRNA constructs for CLEC16A
  source 1..21: mol_type = other DNA
catcttgcggcaaaagtcgtt

SEQ ID NO: 369 - DNA, 21 bp, synthetic construct
  misc_feature 1..21: note = siRNA constructs for CLEC16A
  source 1..21: mol_type = other DNA
aagtcgggccgttacgtgttt

SEQ ID NO: 370 - DNA, 21 bp, synthetic construct
  misc_feature 1..21: note = siRNA constructs for CLEC16A
  source 1..21: mol_type = other DNA
gtcgggccgttacgtgtgctt

SEQ ID NO: 371 - DNA, 21 bp, synthetic construct
  misc_feature 1..21: note = siRNA constructs for CLEC16A
  source 1..21: mol_type = other DNA
catcagtcacgagacctcatt

SEQ ID NO: 372 - DNA, 21 bp, synthetic construct
  misc_feature 1..21: note = siRNA constructs for CLEC16A
  source 1..21: mol_type = other DNA
ataactacgtaaattctattt

SEQ ID NO: 373 - DNA, 21 bp, synthetic construct
  misc_feature 1..21: note = siRNA constructs for CLEC16A
  source 1..21: mol_type = other DNA
ctacgtaaattctatcatctt

SEQ ID NO: 374 - DNA, 21 bp, synthetic construct
  misc_feature 1..21: note = siRNA constructs for CLEC16A
  source 1..21: mol_type = other DNA
atttgacttttctgatgagtt

SEQ ID NO: 375 - DNA, 19 bp, synthetic construct
  misc_feature 1..19: note = siRNA constructs for CLEC16A
  source 1..19: mol_type = other DNA
aacactttcgttaaaactc

SEQ ID NO: 376 - DNA, 21 bp, synthetic construct
  misc_feature 1..21: note = siRNA constructs for CLEC16A
  source 1..21: mol_type = other DNA
caaccacactgtccatttctt

SEQ ID NO: 377 - DNA, 21 bp, synthetic construct
  misc_feature 1..21: note = siRNA constructs for CLEC16A
  source 1..21: mol_type = other DNA
ccacactgtccatttcttttt

SEQ ID NO: 378 - DNA, 21 bp, synthetic construct
  misc_feature 1..21: note = siRNA constructs for CLEC16A
  source 1..21: mol_type = other DNA
tgagcacaccaatgacttttt

SEQ ID NO: 379 - DNA, 21 bp, synthetic construct
  misc_feature 1..21: note = siRNA constructs for CLEC16A
  source 1..21: mol_type = other DNA
tgactttgccctgtacacatt

SEQ ID NO: 380 - DNA, 21 bp, synthetic construct
  misc_feature 1..21: note = siRNA constructs for CLEC16A
  source 1..21: mol_type = other DNA
gccatcaagtttttcaacctt

SEQ ID NO: 381 - DNA, 21 bp, synthetic construct
  misc_feature 1..21: note = siRNA constructs for CLEC16A
  source 1..21: mol_type = other DNA
gtttttcaaccaccctgaatt

SEQ ID NO: 382 - DNA, 21 bp, synthetic construct
  misc_feature 1..21: note = siRNA constructs for CLEC16A
  source 1..21: mol_type = other DNA
ccaccctgaaagcatggtttt

SEQ ID NO: 383 - DNA, 19 bp, synthetic construct
  misc_feature 1..19: note = siRNA constructs for CLEC16A
  source 1..19: mol_type = other DNA
ttgctgtaagaaccataac

SEQ ID NO: 384 - DNA, 21 bp, synthetic construct
  misc_feature 1..21: note = siRNA constructs for CLEC16A
  source 1..21: mol_type = other DNA
gaaccataactttgaatgttt

SEQ ID NO: 385 - DNA, 21 bp, synthetic construct
  misc_feature 1..21: note = siRNA constructs for CLEC16A
  source 1..21: mol_type = other DNA
ctttgaatgtctataaagttt

SEQ ID NO: 386 - DNA, 21 bp, synthetic construct
  misc_feature 1..21: note = siRNA constructs for CLEC16A
  source 1..21: mol_type = other DNA
tgtctataaagtgtcattgtt

SEQ ID NO: 387 - DNA, 21 bp, synthetic construct
  misc_feature 1..21: note = siRNA constructs for CLEC16A
  source 1..21: mol_type = other DNA
agtgtcattggataaccagtt

SEQ ID NO: 388 - DNA, 21 bp, synthetic construct
  misc_feature 1..21: note = siRNA constructs for CLEC16A
  source 1..21: mol_type = other DNA
ccaggccatgctgcactactt

SEQ ID NO: 389 - DNA, 21 bp, synthetic construct
  misc_feature 1..21: note = siRNA constructs for CLEC16A
  source 1..21: mol_type = other DNA
ctgctgttccttacttctctt

SEQ ID NO: 390 - DNA, 21 bp, synthetic construct
  misc_feature 1..21: note = siRNA constructs for CLEC16A
  source 1..21: mol_type = other DNA
tttggtctggttcattgggtt

SEQ ID NO: 391 - DNA, 21 bp, synthetic construct
  misc_feature 1..21: note = siRNA constructs for CLEC16A
  source 1..21: mol_type = other DNA
ctcgatgactgcgtgcagatt

SEQ ID NO: 392 - DNA, 21 bp, synthetic construct
  misc_feature 1..21: note = siRNA constructs for CLEC16A
  source 1..21: mol_type = other DNA
tcggggtaaactgagtgattt

SEQ ID NO: 393 - DNA, 21 bp, synthetic construct
  misc_feature 1..21: note = siRNA constructs for CLEC16A
  source 1..21: mol_type = other DNA
actgagtgatctggtggcatt

SEQ ID NO: 394 - DNA, 21 bp, synthetic construct
  misc_feature 1..21: note = siRNA constructs for CLEC16A
  source 1..21: mol_type = other DNA
tgacatcctgatcatcaactt

SEQ ID NO: 395 - DNA, 21 bp, synthetic construct
  misc_feature 1..21: note = siRNA constructs for CLEC16A
  source 1..21: mol_type = other DNA
ctgtgagttcctcaacgattt

SEQ ID NO: 396 - DNA, 21 bp, synthetic construct
  misc_feature 1..21: note = siRNA constructs for CLEC16A
  source 1..21: mol_type = other DNA
cgatgtgctcactgaccactt

SEQ ID NO: 397 - DNA, 21 bp, synthetic construct
  misc_feature 1..21: note = siRNA constructs for CLEC16A
  source 1..21: mol_type = other DNA
caggctcttcctgcccctctt

SEQ ID NO: 398 - DNA, 21 bp, synthetic construct
  misc_feature 1..21: note = siRNA constructs for CLEC16A
  source 1..21: mol_type = other DNA
gggaggagaacggccgaaatt

SEQ ID NO: 399 - DNA, 21 bp, synthetic construct
  misc_feature 1..21: note = siRNA constructs for CLEC16A
  source 1..21: mol_type = other DNA
cggccgaaaattagcctgctt

SEQ ID NO: 400 - DNA, 21 bp, synthetic construct
  misc_feature 1..21: note = siRNA constructs for CLEC16A
  source 1..21: mol_type = other DNA
aattagcctgccggtgtcttt

SEQ ID NO: 401 - DNA, 21 bp, synthetic construct
  misc_feature 1..21: note = siRNA constructs for CLEC16A
  source 1..21: mol_type = other DNA
ttatacatcatgcaccgcttt

SEQ ID NO: 402 - DNA, 21 bp, synthetic construct
  misc_feature 1..21: note = siRNA constructs for CLEC16A
  source 1..21: mol_type = other DNA
ctcgttagctgaagtcatttt

SEQ ID NO: 403 - DNA, 21 bp, synthetic construct
  misc_feature 1..21: note = siRNA constructs for CLEC16A
  source 1..21: mol_type = other DNA
gtcattctgaatggtgatctt

SEQ ID NO: 404 - DNA, 21 bp, synthetic construct
  misc_feature 1..21: note = siRNA constructs for CLEC16A
  source 1..21: mol_type = other DNA
tggtgatctgtctgagatgtt

SEQ ID NO: 405 - DNA, 21 bp, synthetic construct
  misc_feature 1..21: note = siRNA constructs for CLEC16A
  source 1..21: mol_type = other DNA
gactgaacaggatattcagtt

SEQ ID NO: 406 - DNA, 21 bp, synthetic construct
  misc_feature 1..21: note = siRNA constructs for CLEC16A
  source 1..21: mol_type = other DNA
caggatattcagagaagtttt

SEQ ID NO: 407 - DNA, 21 bp, synthetic construct
  misc_feature 1..21: note = siRNA constructs for CLEC16A
  source 1..21: mol_type = other DNA
gttctgccaagcccagcattt

SEQ ID NO: 408 - DNA, 21 bp, synthetic construct
  misc_feature 1..21: note = siRNA constructs for CLEC16A
  source 1..21: mol_type = other DNA
gcccagcattcggtgcttctt

SEQ ID NO: 409 - DNA, 21 bp, synthetic construct
  misc_feature 1..21: note = siRNA constructs for CLEC16A
  source 1..21: mol_type = other DNA
acccaccgagacactcgagtt

SEQ ID NO: 410 - DNA, 21 bp, synthetic construct
  misc_feature 1..21: note = siRNA constructs for CLEC16A
  source 1..21: mol_type = other DNA
caagcacaagggcaagaggtt

SEQ ID NO: 411 - DNA, 21 bp, synthetic construct
  misc_feature 1..21: note = siRNA constructs for CLEC16A
  source 1..21: mol_type = other DNA
gcacaagggcaagaggcggtt

SEQ ID NO: 412 - DNA, 21 bp, synthetic construct
  misc_feature 1..21: note = siRNA constructs for CLEC16A
  source 1..21: mol_type = other DNA
gggcaagaggcgggtgcaatt

SEQ ID NO: 413 - DNA, 21 bp, synthetic construct
  misc_feature 1..21: note = siRNA constructs for CLEC16A
  source 1..21: mol_type = other DNA
gagacccaactacaaaaactt

SEQ ID NO: 414 - DNA, 21 bp, synthetic construct
  misc_feature 1..21: note = siRNA constructs for CLEC16A
  source 1..21: mol_type = other DNA
ctacaaaaacgttggggaatt

SEQ ID NO: 415 - DNA, 21 bp, synthetic construct
  misc_feature 1..21: note = siRNA constructs for CLEC16A
  source 1..21: mol_type = other DNA
aaacgttggggaagaagaatt

SEQ ID NO: 416 - DNA, 21 bp, synthetic construct
  misc_feature 1..21: note = siRNA constructs for CLEC16A
  source 1..21: mol_type = other DNA
acgttggggaagaagaagatt

SEQ ID NO: 417 - DNA, 21 bp, synthetic construct
  misc_feature 1..21: note = siRNA constructs for CLEC16A
  source 1..21: mol_type = other DNA
gatgaggagaaagggcccatt

SEQ ID NO: 418 - DNA, 21 bp, synthetic construct
  misc_feature 1..21: note = siRNA constructs for CLEC16A
  source 1..21: mol_type = other DNA
agggcccaccgaggatgcctt

SEQ ID NO: 419 - DNA, 21 bp, synthetic construct
  misc_feature 1..21: note = siRNA constructs for CLEC16A
  source 1..21: mol_type = other DNA
gaagacgccgagaaggctatt

SEQ ID NO: 420 - DNA, 21 bp, synthetic construct
  misc_feature 1..21: note = siRNA constructs for CLEC16A
  source 1..21: mol_type = other DNA
gacgccgagaaggctaaagtt

SEQ ID NO: 421 - DNA, 21 bp, synthetic construct
  misc_feature 1..21: note = siRNA constructs for CLEC16A
  source 1..21: mol_type = other DNA
aaggcatcaagacgagtggtt

SEQ ID NO: 422 - DNA, 21 bp, synthetic construct
  misc_feature 1..21: note = siRNA constructs for CLEC16A
  source 1..21: mol_type = other DNA
ggcatcaagacgagtggggtt

SEQ ID NO: 423 - DNA, 21 bp, synthetic construct
  misc_feature 1..21: note = siRNA constructs for CLEC16A
  source 1..21: mol_type = other DNA
gacgagtggggagagtgaatt

SEQ ID NO: 424 - DNA, 21 bp, synthetic construct
  misc_feature 1..21: note = siRNA constructs for CLEC16A
  source 1..21: mol_type = other DNA
gctctcagagctggccgcctt

SEQ ID NO: 425 - DNA, 21 bp, synthetic construct
  misc_feature 1..21: note = siRNA constructs for CLEC16A
  source 1..21: mol_type = other DNA
caccacggacgaggagaaatt

SEQ ID NO: 426 - DNA, 21 bp, synthetic construct
  misc_feature 1..21: note = siRNA constructs for CLEC16A
  source 1..21: mol_type = other DNA
aagcgccgccgccacctgctt

SEQ ID NO: 427 - DNA, 21 bp, synthetic construct
  misc_feature 1..21: note = siRNA constructs for CLEC16A
  source 1..21: mol_type = other DNA
gcgccgccgccacctgctctt

SEQ ID NO: 428 - DNA, 21 bp, synthetic construct
  misc_feature 1..21: note = siRNA constructs for CLEC16A
  source 1..21: mol_type = other DNA
taaaggcatggatcctgaatt

SEQ ID NO: 429 - DNA, 21 bp, synthetic construct
  misc_feature 1..21: note = siRNA constructs for CLEC16A
  source 1..21: mol_type = other DNA
aggcatggatcctgaaaaatt

SEQ ID NO: 430 - DNA, 21 bp, synthetic construct
  misc_feature 1..21: note = siRNA constructs for CLEC16A
  source 1..21: mol_type = other DNA
aaattagagcgaatccagctt

SEQ ID NO: 431 - DNA, 21 bp, synthetic construct
  misc_feature 1..21: note = siRNA constructs for CLEC16A
  source 1..21: mol_type = other DNA
attagagcgaatccagctctt

SEQ ID NO: 432 - DNA, 21 bp, synthetic construct
  misc_feature 1..21: note = siRNA constructs for CLEC16A
  source 1..21: mol_type = other DNA
tccagctccccgtgccaaatt

SEQ ID NO: 433 - DNA, 21 bp, synthetic construct
  misc_feature 1..21: note = siRNA constructs for CLEC16A
  source 1..21: mol_type = other DNA
atgcggccgagaagaccactt

SEQ ID NO: 434 - DNA, 21 bp, synthetic construct
  misc_feature 1..21: note = siRNA constructs for CLEC16A
  source 1..21: mol_type = other DNA
gaccacctacaaccacccgtt

SEQ ID NO: 435 - DNA, 21 bp, synthetic construct
  misc_feature 1..21: note = siRNA constructs for CLEC16A
  source 1..21: mol_type = other DNA
ccacccgctagctgaaagatt

SEQ ID NO: 436 - DNA, 21 bp, synthetic construct
  misc_feature 1..21: note = siRNA constructs for CLEC16A
  source 1..21: mol_type = other DNA
cgctgcccagccagatgggtt

SEQ ID NO: 437 - DNA, 21 bp, synthetic construct
  misc_feature 1..21: note = siRNA constructs for CLEC16A
  source 1..21: mol_type = other DNA
gatccggctggcgacgctgtt

SEQ ID NO: 438 - DNA, 21 bp, synthetic construct
  misc_feature 1..21: note = siRNA constructs for CLEC16A
  source 1..21: mol_type = other DNA
gcagcaagtcctgatgagttt

SEQ ID NO: 439 - DNA, 21 bp, synthetic construct
  misc_feature 1..21: note = siRNA constructs for CLEC16A
  source 1..21: mol_type = other DNA
gtcctgatgagtgctggcttt

SEQ ID NO: 440 - DNA, 21 bp, synthetic construct
  misc_feature 1..21: note = siRNA constructs for CLEC16A
  source 1..21: mol_type = other DNA
ggacgtgcacctggcctgctt

SEQ ID NO: 441 - DNA, 21 bp, synthetic construct
  misc_feature 1..21: note = siRNA constructs for CLEC16A
  source 1..21: mol_type = other DNA
gaaagtgttcaccttgtactt

SEQ ID NO: 442 - DNA, 21 bp, synthetic construct
  misc_feature 1..21: note = siRNA constructs for CLEC16A
  source 1..21: mol_type = other DNA
agtgttcaccttgtacgactt

SEQ ID NO: 443 - DNA, 21 bp, synthetic construct
  misc_feature 1..21: note = siRNA constructs for CLEC16A
  source 1..21: mol_type = other DNA
gggagaagacatttttttgtt

SEQ ID NO: 444 - DNA, 21 bp, synthetic construct
  misc_feature 1..21: note = siRNA constructs for CLEC16A
  source 1..21: mol_type = other DNA
gacatttttttggacatgttt

SEQ ID NO: 445 - DNA, 21 bp, synthetic construct
  misc_feature 1..21: note = siRNA constructs for CLEC16A
  source 1..21: mol_type = other DNA
gatgagtataggagcatgatt

SEQ ID NO: 446 - DNA, 21 bp, synthetic construct
  misc_feature 1..21: note = siRNA constructs for CLEC16A
  source 1..21: mol_type = other DNA
gcccatgaacgtggaatattt

SEQ ID NO: 447 - DNA, 21 bp, synthetic construct
  misc_feature 1..21: note = siRNA constructs for CLEC16A
  source 1..21: mol_type = other DNA
tatctcatgatggacgccttt

SEQ ID NO: 448 - DNA, 21 bp, synthetic construct
  misc_feature 1..21: note = siRNA constructs for CLEC16A
  source 1..21: mol_type = other DNA
gactgatgatgtcctggattt

SEQ ID NO: 449 - DNA, 21 bp, synthetic construct
  misc_feature 1..21: note = siRNA constructs for CLEC16A
  source 1..21: mol_type = other DNA
taacagcgacttgattgcatt

SEQ ID NO: 450 - DNA, 21 bp, synthetic construct
  misc_feature 1..21: note = siRNA constructs for CLEC16A
  source 1..21: mol_type = other DNA
cagcgacttgattgcatgttt

SEQ ID NO: 451 - DNA, 21 bp, synthetic construct
  misc_feature 1..21: note = siRNA constructs for CLEC16A
  source 1..21: mol_type = other DNA
ggatggcggcatggtccagtt

SEQ ID NO: 452 - DNA, 21 bp, synthetic construct
  misc_feature 1..21: note = siRNA constructs for CLEC16A
  source 1..21: mol_type = other DNA
gtttgcaggcctattgcagtt

SEQ ID NO: 453 - DNA, 21 bp, synthetic construct
  misc_feature 1..21: note = siRNA constructs for CLEC16A
  source 1..21: mol_type = other DNA
catcaccatccacaagccttt

SEQ ID NO: 454 - DNA, 21 bp, synthetic construct
  misc_feature 1..21: note = siRNA constructs for CLEC16A
  source 1..21: mol_type = other DNA
gcctgcgtccagcccccattt

SEQ ID NO: 455 - DNA, 21 bp, synthetic construct
  misc_feature 1..21: note = siRNA constructs for CLEC16A
  source 1..21: mol_type = other DNA
gcccttccccatcctccagtt

SEQ ID NO: 456 - DNA, 21 bp, synthetic construct
  misc_feature 1..21: note = siRNA constructs for CLEC16A
  source 1..21: mol_type = other DNA
gcagcgcctggccaaaggctt

SEQ ID NO: 457 - DNA, 21 bp, synthetic construct
  misc_feature 1..21: note = siRNA constructs for CLEC16A
  source 1..21: mol_type = other DNA
aggccgcatccaggcaaggtt

SEQ ID NO: 458 - DNA, 21 bp, synthetic construct
  misc_feature 1..21: note = siRNA constructs for CLEC16A
  source 1..21: mol_type = other DNA
ggcgcatgaagatgcagagtt

SEQ ID NO: 459 - DNA, 21 bp, synthetic construct
  misc_feature 1..21: note = siRNA constructs for CLEC16A
  source 1..21: mol_type = other DNA
gatgcagagaatagctgcctt

SEQ ID NO: 460 - DNA, 21 bp, synthetic construct
  misc_feature 1..21: note = siRNA constructs for CLEC16A
  source 1..21: mol_type = other DNA
tagctgccctcctggaccttt

SEQ ID NO: 461 - DNA, 21 bp, synthetic construct
  misc_feature 1..21: note = siRNA constructs for CLEC16A
  source 1..21: mol_type = other DNA
tccagcccaccactgaagttt

SEQ ID NO: 462 - DNA, 21 bp, synthetic construct
  misc_feature 1..21: note = siRNA constructs for CLEC16A
  source 1..21: mol_type = other DNA
gtcctggggtttggactcgtt

SEQ ID NO: 463 - DNA, 21 bp, synthetic construct
  misc_feature 1..21: note = siRNA constructs for CLEC16A
  source 1..21: mol_type = other DNA
ggtgccaggcttcgccgtgtt

SEQ ID NO: 464 - DNA, 21 bp, synthetic construct
  misc_feature 1..21: note = siRNA constructs for CLEC16A
  source 1..21: mol_type = other DNA
accagcacagctccccgtctt

SEQ ID NO: 465 - DNA, 21 bp, synthetic construct
  misc_feature 1..21: note = siRNA constructs for CLEC16A
  source 1..21: mol_type = other DNA
ctgcctaagcctcaccttctt

SEQ ID NO: 466 - DNA, 21 bp, synthetic construct
  misc_feature 1..21: note = siRNA constructs for CLEC16A
  source 1..21: mol_type = other DNA
gcctcaccttcctgaccagtt

SEQ ID NO: 467 - DNA, 21 bp, synthetic construct
  misc_feature 1..21: note = siRNA constructs for CLEC16A
  source 1..21: mol_type = other DNA
gcagactctaagcccagcatt

SEQ ID NO: 468 - DNA, 21 bp, synthetic construct
  misc_feature 1..21: note = siRNA constructs for CLEC16A
  source 1..21: mol_type = other DNA
gcccagcaagaacgtggcctt

SEQ ID NO: 469 - DNA, 21 bp, synthetic construct
  misc_feature 1..21: note = siRNA constructs for CLEC16A
  source 1..21: mol_type = other DNA
gaacgtggccaggagcgcatt

SEQ ID NO: 470 - DNA, 21 bp, synthetic construct
  misc_feature 1..21: note = siRNA constructs for CLEC16A
  source 1..21: mol_type = other DNA
tcgctgacccttgtccccctt

SEQ ID NO: 471 - DNA, 21 bp, synthetic construct
  misc_feature 1..21: note = siRNA constructs for CLEC16A
  source 1..21: mol_type = other DNA
gacacactgggagcacccatt

SEQ ID NO: 472 - DNA, 21 bp, synthetic construct
  misc_feature 1..21: note = siRNA constructs for CLEC16A
  source 1..21: mol_type = other DNA
ccacctatccctgcgctcctt

SEQ ID NO: 473 - DNA, 21 bp, synthetic construct
  misc_feature 1..21: note = siRNA constructs for CLEC16A
  source 1..21: mol_type = other DNA
tgggaagaagccccacgtttt

SEQ ID NO: 474 - DNA, 21 bp, synthetic construct
  misc_feature 1..21: note = siRNA constructs for CLEC16A
  source 1..21: mol_type = other DNA
gaagccccacgttgtcctttt

SEQ ID NO: 475 - DNA, 21 bp, synthetic construct
  misc_feature 1..21: note = siRNA constructs for CLEC16A
  source 1..21: mol_type = other DNA
ttcctttttcactttgcattt

SEQ ID NO: 476 - DNA, 21 bp, synthetic construct
  misc_feature 1..21: note = siRNA constructs for CLEC16A
  source 1..21: mol_type = other DNA
tgcagatgactgcaccggctt

SEQ ID NO: 477 - DNA, 21 bp, synthetic construct
  misc_feature 1..21: note = siRNA constructs for CLEC16A
  source 1..21: mol_type = other DNA
gaattggaccaggtcactgtt

SEQ ID NO: 478 - DNA, 21 bp, synthetic construct
  misc_feature 1..21: note = siRNA constructs for CLEC16A
  source 1..21: mol_type = other DNA
ttggaccaggtcactgtactt

SEQ ID NO: 479 - DNA, 21 bp, synthetic construct
  misc_feature 1..21: note = siRNA constructs for CLEC16A
  source 1..21: mol_type = other DNA
atttgtagaaaagcagacttt

SEQ ID NO: 480 - DNA, 21 bp, synthetic construct
  misc_feature 1..21: note = siRNA constructs for CLEC16A
  source 1..21: mol_type = other DNA
aagcagacttagataaacatt

SEQ ID NO: 481 - DNA, 21 bp, synthetic construct
  misc_feature 1..21: note = siRNA constructs for CLEC16A
  source 1..21: mol_type = other DNA
aagaagtccaaaaacatcatt

SEQ ID NO: 482 - DNA, 21 bp, synthetic construct
  misc_feature 1..21: note = siRNA constructs for CLEC16A
  source 1..21: mol_type = other DNA
gaagtccaaaaacatcaactt

SEQ ID NO: 483 - DNA, 21 bp, synthetic construct
  misc_feature 1..21: note = siRNA constructs for CLEC16A
  source 1..21: mol_type = other DNA
aaacatcaacactaaggtttt

SEQ ID NO: 484 - DNA, 21 bp, synthetic construct
  misc_feature 1..21: note = siRNA constructs for CLEC16A
  source 1..21: mol_type = other DNA
acatcaacactaaggtttgtt

SEQ ID NO: 485 - DNA, 21 bp, synthetic construct
  misc_feature 1..21: note = siRNA constructs for CLEC16A
  source 1..21: mol_type = other DNA
cactaaggtttgatgtcattt

SEQ ID NO: 486 - DNA, 21 bp, synthetic construct
  misc_feature 1..21: note = siRNA constructs for CLEC16A
  source 1..21: mol_type = other DNA
gtgattctttgttactcactt

SEQ ID NO: 487 - DNA, 21 bp, synthetic construct
  misc_feature 1..21: note = siRNA constructs for CLEC16A
  source 1..21: mol_type = other DNA
ctctgcaagcctgtggaattt

SEQ ID NO: 488 - DNA, 21 bp, synthetic construct
  misc_feature 1..21: note = siRNA constructs for CLEC16A
  source 1..21: mol_type = other DNA
gcctgtggaataatgaagttt

SEQ ID NO: 489 - DNA, 21 bp, synthetic construct
  misc_feature 1..21: note = siRNA constructs for CLEC16A
  source 1..21: mol_type = other DNA
taatgaagtaccttcctggtt

SEQ ID NO: 490 - DNA, 21 bp, synthetic construct
  misc_feature 1..21: note = siRNA constructs for CLEC16A
  source 1..21: mol_type = other DNA
tgaagtaccttcctggaaatt

SEQ ID NO: 491 - DNA, 21 bp, synthetic construct
  misc_feature 1..21: note = siRNA constructs for CLEC16A
  source 1..21: mol_type = other DNA
agtttggattattttttaatt

SEQ ID NO: 492 - DNA, 21 bp, synthetic construct
  misc_feature 1..21: note = siRNA constructs for CLEC16A
  source 1..21: mol_type = other DNA
acaaaaacaagggagatactt

SEQ ID NO: 493 - DNA, 21 bp, synthetic construct
  misc_feature 1..21: note = siRNA constructs for CLEC16A
  source 1..21: mol_type = other DNA
aaacaagggagatacatgttt

SEQ ID NO: 494 - DNA, 21 bp, synthetic construct
  misc_feature 1..21: note = siRNA constructs for CLEC16A
  source 1..21: mol_type = other DNA
acaagggagatacatgtattt

SEQ ID NO: 495 - DNA, 21 bp, synthetic construct
  misc_feature 1..21: note = siRNA constructs for CLEC16A
  source 1..21: mol_type = other DNA
gggagatacatgtattctctt

SEQ ID NO: 496 - DNA, 21 bp, synthetic construct
  misc_feature 1..21: note = siRNA constructs for CLEC16A
  source 1..21: mol_type = other DNA
tggttttctgctatagcagtt

SEQ ID NO: 497 - DNA, 19 bp, synthetic construct
  misc_feature 1..19: note = siRNA constructs for CLEC16A
  source 1..19: mol_type = other DNA
gaaggaaccagttgggacc

SEQ ID NO: 498 - DNA, 21 bp, synthetic construct
  misc_feature 1..21: note = siRNA constructs for CLEC16A
  source 1..21: mol_type = other DNA
ggaaccagttgggaccgtgtt

SEQ ID NO: 499 - DNA, 21 bp, synthetic construct
  misc_feature 1..21: note = siRNA constructs for CLEC16A
  source 1..21: mol_type = other DNA
ccagttgggaccgtgaagatt

SEQ ID NO: 500 - DNA, 21 bp, synthetic construct
  misc_feature 1..21: note = siRNA constructs for CLEC16A
  source 1..21: mol_type = other DNA
gactcccgaccctgtggcctt

SEQ ID NO: 501 - DNA, 21 bp, synthetic construct
  misc_feature 1..21: note = siRNA constructs for CLEC16A
  source 1..21: mol_type = other DNA
aggaagacaccctctacgttt

SEQ ID NO: 502 - DNA, 21 bp, synthetic construct
  misc_feature 1..21: note = siRNA constructs for CLEC16A
  source 1..21: mol_type = other DNA
gacaccctctacgtcaccttt

SEQ ID NO: 503 - DNA, 21 bp, synthetic construct
  misc_feature 1..21: note = siRNA constructs for CLEC16A
  source 1..21: mol_type = other DNA
gggcatggtgtccccagagtt

SEQ ID NO: 504 - DNA, 21 bp, synthetic construct
  misc_feature 1..21: note = siRNA constructs for CLEC16A
  source 1..21: mol_type = other DNA
tatggaaaccccatggttctt

SEQ ID NO: 505 - DNA, 21 bp, synthetic construct
  misc_feature 1..21: note = siRNA constructs for CLEC16A
  source 1..21: mol_type = other DNA
accccatggttcccttccctt

SEQ ID NO: 506 - DNA, 21 bp, synthetic construct
  misc_feature 1..21: note = siRNA constructs for CLEC16A
  source 1..21: mol_type = other DNA
gcaaggacccagatgcatctt

SEQ ID NO: 507 - DNA, 21 bp, synthetic construct
  misc_feature 1..21: note = siRNA constructs for CLEC16A
  source 1..21: mol_type = other DNA
ggacccagatgcatcagactt

SEQ ID NO: 508 - DNA, 21 bp, synthetic construct
  misc_feature 1..21: note = siRNA constructs for CLEC16A
  source 1..21: mol_type = other DNA
gatgttcctttctactcggtt

SEQ ID NO: 509 - DNA, 21 bp, synthetic construct
  misc_feature 1..21: note = siRNA constructs for CLEC16A
  source 1..21: mol_type = other DNA
gtccaccagggccagcggctt

SEQ ID NO: 510 - DNA, 21 bp, synthetic construct
  misc_feature 1..21: note = siRNA constructs for CLEC16A
  source 1..21: mol_type = other DNA
ctttcctccagacctgccatt

SEQ ID NO: 511 - DNA, 21 bp, synthetic construct
  misc_feature 1..21: note = siRNA constructs for CLEC16A
  source 1..21: mol_type = other DNA
gaacatggtctctgtctcctt

SEQ ID NO: 512 - DNA, 21 bp, synthetic construct
  misc_feature 1..21: note = siRNA constructs for CLEC16A
  source 1..21: mol_type = other DNA
catggtctctgtctcctcgtt

SEQ ID NO: 513 - DNA, 21 bp, synthetic construct
  misc_feature 1..21: note = siRNA constructs for CLEC16A
  source 1..21: mol_type = other DNA
ggcctgccgagggcagttttt

SEQ ID NO: 514 - DNA, 21 bp, synthetic construct
  misc_feature 1..21: note = siRNA constructs for CLEC16A
  source 1..21: mol_type = other DNA
cctcatgaaggaaacacagtt

SEQ ID NO: 515 - DNA, 21 bp, synthetic construct
  misc_feature 1..21: note = siRNA constructs for CLEC16A
  source 1..21: mol_type = other DNA
ggaaacacagtcctgccaatt

SEQ ID NO: 516 - DNA, 21 bp, synthetic construct
  misc_feature 1..21: note = siRNA constructs for CLEC16A
  source 1..21: mol_type = other DNA
acacagtcctgccaaggagtt

SEQ ID NO: 517 - DNA, 21 bp, synthetic construct
  misc_feature 1..21: note = siRNA constructs for CLEC16A
  source 1..21: mol_type = other DNA
ggagggggagtggcgcccatt

SEQ ID NO: 518 - DNA, 21 bp, synthetic construct
  misc_feature 1..21: note = siRNA constructs for CLEC16A
  source 1..21: mol_type = other DNA
gccctctgggtagctgtgctt

SEQ ID NO: 519 - DNA, 21 bp, synthetic construct
  misc_feature 1..21: note = siRNA constructs for CLEC16A
  source 1..21: mol_type = other DNA
ggacctttgcttccatagatt

SEQ ID NO: 520 - DNA, 21 bp, synthetic construct
  misc_feature 1..21: note = siRNA constructs for CLEC16A
  source 1..21: mol_type = other DNA
aacgcacagctcagaaaggtt

SEQ ID NO: 521 - DNA, 21 bp, synthetic construct
  misc_feature 1..21: note = siRNA constructs for CLEC16A
  source 1..21: mol_type = other DNA
tccctggaagagaaggaagtt

SEQ ID NO: 522 - DNA, 21 bp, synthetic construct
  misc_feature 1..21: note = siRNA constructs for CLEC16A
  source 1..21: mol_type = other DNA
gagaaggaaggcagggtggtt

SEQ ID NO: 523 - DNA, 21 bp, synthetic construct
  misc_feature 1..21: note = siRNA constructs for CLEC16A
  source 1..21: mol_type = other DNA
ggaaggcagggtggagcggtt

SEQ ID NO: 524 - DNA, 21 bp, synthetic construct
  misc_feature 1..21: note = siRNA constructs for CLEC16A
  source 1..21: mol_type = other DNA
ggcagggtggagcggggggtt

SEQ ID NO: 525 - DNA, 21 bp, synthetic construct
  misc_feature 1..21: note = siRNA constructs for CLEC16A
  source 1..21: mol_type = other DNA
gaccatcatggagagaaggtt

SEQ ID NO: 526 - DNA, 21 bp, synthetic construct
  misc_feature 1..21: note = siRNA constructs for CLEC16A
  source 1..21: mol_type = other DNA
ggaccacagcatcaggagatt

SEQ ID NO: 527 - DNA, 21 bp, synthetic construct
  misc_feature 1..21: note = siRNA constructs for CLEC16A
  source 1..21: mol_type = other DNA
ttttttaagagcaagagggtt

SEQ ID NO: 528 - DNA, 21 bp, synthetic construct
  misc_feature 1..21: note = siRNA constructs for CLEC16A
  source 1..21: mol_type = other DNA
gagcaagaggggtagagagtt

SEQ ID NO: 529 - DNA, 21 bp, synthetic construct
  misc_feature 1..21: note = siRNA constructs for CLEC16A
  source 1..21: mol_type = other DNA
gaggggtagagaggatcaatt

SEQ ID NO: 530 - DNA, 21 bp, synthetic construct
  misc_feature 1..21: note = siRNA constructs for CLEC16A
  source 1..21: mol_type = other DNA
gctggccctggctggagattt

SEQ ID NO: 531 - DNA, 21 bp, synthetic construct
  misc_feature 1..21: note = siRNA constructs for CLEC16A
  source 1..21: mol_type = other DNA
atgactctgtctgtggggctt

SEQ ID NO: 532 - DNA, 21 bp, synthetic construct
  misc_feature 1..21: note = siRNA constructs for CLEC16A
  source 1..21: mol_type = other DNA
ggcaagtggctgccccacctt

SEQ ID NO: 533 - DNA, 21 bp, synthetic construct
  misc_feature 1..21: note = siRNA constructs for CLEC16A
  source 1..21: mol_type = other DNA
gtggctgccccaccccaagtt

SEQ ID NO: 534 - DNA, 21 bp, synthetic construct
  misc_feature 1..21: note = siRNA constructs for CLEC16A
  source 1..21: mol_type = other DNA
cagcctgcagctcactccatt

SEQ ID NO: 535 - DNA, 21 bp, synthetic construct
  misc_feature 1..21: note = siRNA constructs for CLEC16A
  source 1..21: mol_type = other DNA
gtctctgggctccaactggtt

SEQ ID NO: 536 - DNA, 21 bp, synthetic construct
  misc_feature 1..21: note = siRNA constructs for CLEC16A
  source 1..21: mol_type = other DNA
ctggtcttgtaaccactgatt

SEQ ID NO: 537 - DNA, 21 bp, synthetic construct
  misc_feature 1..21: note = siRNA constructs for CLEC16A
  source 1..21: mol_type = other DNA
ccactgagcactgaaggagtt

SEQ ID NO: 538 - DNA, 21 bp, synthetic construct
  misc_feature 1..21: note = siRNA constructs for CLEC16A
  source 1..21: mol_type = other DNA
ggagagaggtcttggtcagtt

SEQ ID NO: 539 - DNA, 21 bp, synthetic construct
  misc_feature 1..21: note = siRNA constructs for CLEC16A
  source 1..21: mol_type = other DNA
aggtgactgggaggaccagtt

SEQ ID NO: 540 - DNA, 21 bp, synthetic construct
  misc_feature 1..21: note = siRNA constructs for CLEC16A
  source 1..21: mol_type = other DNA
aagacactgctcagggcagtt

SEQ ID NO: 541 - DNA, 21 bp, synthetic construct
  misc_feature 1..21: note = siRNA constructs for CLEC16A
  source 1..21: mol_type = other DNA
gacactgctcagggcagggtt

SEQ ID NO: 542 - DNA, 21 bp, synthetic construct
  misc_feature 1..21: note = siRNA constructs for CLEC16A
  source 1..21: mol_type = other DNA
gaaaagggcagtccccatgtt

SEQ ID NO: 543 - DNA, 21 bp, synthetic construct
  misc_feature 1..21: note = siRNA constructs for CLEC16A
  source 1..21: mol_type = other DNA
aagggcagtccccatgtggtt

SEQ ID NO: 544 - DNA, 21 bp, synthetic construct
  misc_feature 1..21: note = siRNA constructs for CLEC16A
  source 1..21: mol_type = other DNA
gggcagtccccatgtgggctt

SEQ ID NO: 545 - DNA, 21 bp, synthetic construct
  misc_feature 1..21: note = siRNA constructs for CLEC16A
  source 1..21: mol_type = other DNA
cttgcgtctgggggacacctt

SEQ ID NO: 546 - DNA, 21 bp, synthetic construct
  misc_feature 1..21: note = siRNA constructs for CLEC16A
  source 1..21: mol_type = other DNA
ccagggctcccagaagctttt

SEQ ID NO: 547 - DNA, 21 bp, synthetic construct
  misc_feature 1..21: note = siRNA constructs for CLEC16A
  source 1..21: mol_type = other DNA
ggaggtctgggagccagcctt

SEQ ID NO: 548 - DNA, 21 bp, synthetic construct
  misc_feature 1..21: note = siRNA constructs for CLEC16A
  source 1..21: mol_type = other DNA
aggaccccacctcacccagtt

SEQ ID NO: 549 - DNA, 21 bp, synthetic construct
  misc_feature 1..21: note = siRNA constructs for CLEC16A
  source 1..21: mol_type = other DNA
acctcatagctggggcgcttt

SEQ ID NO: 550 - DNA, 21 bp, synthetic construct
  misc_feature 1..21: note = siRNA constructs for CLEC16A
  source 1..21: mol_type = other DNA
gagccaggagtgtgggaagtt

SEQ ID NO: 551 - DNA, 21 bp, synthetic construct
  misc_feature 1..21: note = siRNA constructs for CLEC16A
  source 1..21: mol_type = other DNA
ggcccacagtgggggctgttt

SEQ ID NO: 552 - DNA, 21 bp, synthetic construct
  misc_feature 1..21: note = siRNA constructs for CLEC16A
  source 1..21: mol_type = other DNA
gtccctgcagagagcactttt

SEQ ID NO: 553 - DNA, 21 bp, synthetic construct
  misc_feature 1..21: note = siRNA constructs for CLEC16A
  source 1..21: mol_type = other DNA
ctcccaaaccttggctttgtt

SEQ ID NO: 554 - DNA, 21 bp, synthetic construct
  misc_feature 1..21: note = siRNA constructs for CLEC16A
  source 1..21: mol_type = other DNA
tattgttgtggaggtgtgctt

SEQ ID NO: 555 - DNA, 21 bp, synthetic construct
  misc_feature 1..21: note = siRNA constructs for CLEC16A
  source 1..21: mol_type = other DNA
ggtacctgtcccagcaggttt

SEQ ID NO: 556 - DNA, 21 bp, synthetic construct
  misc_feature 1..21: note = siRNA constructs for CLEC16A
  source 1..21: mol_type = other DNA
cccagacacttgcgttcactt

SEQ ID NO: 557 - DNA, 21 bp, synthetic construct
  misc_feature 1..21: note = siRNA constructs for CLEC16A
  source 1..21: mol_type = other DNA
gcaacctaaggggcaggtgtt

SEQ ID NO: 558 - DNA, 19 bp, synthetic construct
  misc_feature 1..19: note = siRNA constructs for CLEC16A
  source 1..19: mol_type = other DNA
cctaaggggcaggtgaaga

SEQ ID NO: 559 - DNA, 21 bp, synthetic construct
  misc_feature 1..21: note = siRNA constructs for CLEC16A
  source 1..21: mol_type = other DNA
ggggcaggtgaagaagcgctt

SEQ ID NO: 560 - DNA, 21 bp, synthetic construct
  misc_feature 1..21: note = siRNA constructs for CLEC16A
  source 1..21: mol_type = other DNA
gaagcgcagccctgccagatt

SEQ ID NO: 561 - DNA, 21 bp, synthetic construct
  misc_feature 1..21: note = siRNA constructs for CLEC16A
  source 1..21: mol_type = other DNA
gcgcagccctgccagacgctt

SEQ ID NO: 562 - DNA, 21 bp, synthetic construct
  misc_feature 1..21: note = siRNA constructs for CLEC16A
  source 1..21: mol_type = other DNA
ggtctctgagatgcaccgttt

SEQ ID NO: 563 - DNA, 21 bp, synthetic construct
  misc_feature 1..21: note = siRNA constructs for CLEC16A
  source 1..21: mol_type = other DNA
aaaggcgtggggtgaactgtt

SEQ ID NO: 564 - DNA, 21 bp, synthetic construct
  misc_feature 1..21: note = siRNA constructs for CLEC16A
  source 1..21: mol_type = other DNA
aggcgtggggtgaactgattt

SEQ ID NO: 565 - DNA, 21 bp, synthetic construct
  misc_feature 1..21: note = siRNA constructs for CLEC16A
  source 1..21: mol_type = other DNA
ctgattttgatcttcttgttt

SEQ ID NO: 566 - DNA, 21 bp, synthetic construct
  misc_feature 1..21: note = siRNA constructs for CLEC16A
  source 1..21: mol_type = other DNA
taaataaatctgaagcatttt

SEQ ID NO: 567 - DNA, 21 bp, synthetic construct
  misc_feature 1..21: note = siRNA constructs for CLEC16A
  source 1..21: mol_type = other DNA
atctgaagcatttaatgtatt

SEQ ID NO: 568 - DNA, 21 bp, synthetic construct
  misc_feature 1..21: note = siRNA constructs for CLEC16A
  source 1..21: mol_type = other DNA
gcatttaatgtagtcatcttt

SEQ ID NO: 569 - DNA, 21 bp, synthetic construct
  misc_feature 1..21: note = siRNA constructs for CLEC16A
  source 1..21: mol_type = other DNA
tgtagtcatcttgacattgtt

SEQ ID NO: 570 - DNA, 21 bp, synthetic construct
  misc_feature 1..21: note = siRNA constructs for CLEC16A
  source 1..21: mol_type = other DNA
aaatatgtaaataattttttt

SEQ ID NO: 571 - DNA, 21 bp, synthetic construct
  misc_feature 1..21: note = siRNA constructs for CLEC16A
  source 1..21: mol_type = other DNA
atatgtaaataatttttgttt

SEQ ID NO: 572 - DNA, 21 bp, synthetic construct
  misc_feature 1..21: note = siRNA constructs for CLEC16A
  source 1..21: mol_type = other DNA
ataatttttgtcccagtgatt

SEQ ID NO: 573 - DNA, 21 bp, synthetic construct
  misc_feature 1..21: note = siRNA constructs for CLEC16A
  source 1..21: mol_type = other DNA
tttttgtcccagtgagaactt

SEQ ID NO: 574 - DNA, 21 bp, synthetic construct
  misc_feature 1..21: note = siRNA constructs for CLEC16A
  source 1..21: mol_type = other DNA
ccgagggttagaaaacctctt

SEQ ID NO: 575 - DNA, 21 bp, synthetic construct
  misc_feature 1..21: note = siRNA constructs for CLEC16A
  source 1..21: mol_type = other DNA
aacctcgatgcctctgagctt

SEQ ID NO: 576 - DNA, 21 bp, synthetic construct
  misc_feature 1..21: note = siRNA constructs for CLEC16A
  source 1..21: mol_type = other DNA
cctcgatgcctctgagccttt

SEQ ID NO: 577 - DNA, 21 bp, synthetic construct
  misc_feature 1..21: note = siRNA constructs for CLEC16A
  source 1..21: mol_type = other DNA
gtacctgctttcgccagcatt

SEQ ID NO: 578 - DNA, 21 bp, synthetic construct
  misc_feature 1..21: note = siRNA constructs for CLEC16A
  source 1..21: mol_type = other DNA
cacgagggtggaaatgaaatt

SEQ ID NO: 579 - DNA, 21 bp, synthetic construct
  misc_feature 1..21: note = siRNA constructs for CLEC16A
  source 1..21: mol_type = other DNA
atgaaaactggaacttccttt

SEQ ID NO: 580 - DNA, 21 bp, synthetic construct
  misc_feature 1..21: note = siRNA constructs for CLEC16A
  source 1..21: mol_type = other DNA
aactggaacttccttgtaatt

SEQ ID NO: 581 - DNA, 21 bp, synthetic construct
  misc_feature 1..21: note = siRNA constructs for CLEC16A
  source 1..21: mol_type = other DNA
ctggaacttccttgtaaattt

SEQ ID NO: 582 - DNA, 21 bp, synthetic construct
  misc_feature 1..21: note = siRNA constructs for CLEC16A
  source 1..21: mol_type = other DNA
cttccttgtaaatttaaactt

(19) United States
(12) Patent Application Publication
(10) Pub. No.: US 2023/0304018 A1
(43) Pub. Date: Sep. 28, 2023

(54) COMPOSITIONS AND METHODS FOR ENHANCING WEIGHT LOSS

(71) Applicant: The Children's Hospital of Philadelphia, Philadelphia, PA (US)

(72) Inventors: Hakon Hakonarson, Malvern, PA (US); Rahul Pandey, Wallingford, PA (US); Marina Bakay, Parkesburg, PA (US); Heather Hain, Ambler, PA (US)

(73) Assignee: The Children's Hospital of Philadelphia, Philadelphia, PA (US)

(21) Appl. No.: 18/047,189

(22) Filed: Oct. 17, 2022

Related U.S. Application Data
(63) Continuation of application No. 16/644,347, filed on Mar. 4, 2020, now abandoned.
(60) Provisional application No. 62/555,631, filed on Sep. 7, 2017.

(51) Int. Cl. C12N 15/113; A61P 3/04; A61K 45/06

(52) U.S. Cl. CPC C12N 15/1138; A61P 3/04; A61K 45/06

(57) ABSTRACT

Compositions and methods for the detection and treatment of obesity and other neurological disorders are provided.

### CROSS-REFERENCE TO RELATED APPLICATIONS

The present application is a continuation application of U.S. application Ser. No. 16/644,347, filed Mar. 4, 2020, which is a § 371 of International Application No. PCT/US2018/050027, filed Sep. 7, 2018, which claims the benefit of U.S. Provisional Application No. 62/555,631, filed Sep. 7, 2017. The entire disclosure of each of the aforesaid applications is incorporated herein by reference in the present application.

### INCORPORATION BY REFERENCE OF MATERIAL SUBMITTED IN ELECTRONIC FORM

The contents of the electronic sequence listing (CHOP-P06519US02.xml; Size: 745,472 bytes; and Date of Creation: Oct. 17, 2022) is herein incorporated by reference in its entirety.

### FIELD OF THE INVENTION

This invention relates to the fields of genetics and metabolism. More specifically, the invention provides compositions and methods for enhancing weight loss in subjects in need thereof by modulating CLEC16A expression levels.

### BACKGROUND OF THE INVENTION

Several publications and patent documents are cited throughout the specification in order to describe the state of the art to which this invention pertains. Each of these citations is incorporated by reference herein as though set forth in full.

Clec16a has been identified as a disease susceptibility gene for type 1 diabetes, multiple sclerosis, and adrenal dysfunction, to name a few (14 autoimmune diseases total have been associated by us and others to this autoimmune gene). Clec16a is a membrane-associated endosomal protein that interacts with E3 ubiquitin ligase Nrdp1. Loss of Clec16a leads to an increase in the Nrdp1 target Parkin, a master regulator of mitophagy. Islets from mice with pancreas-specific deletion of Clec16a have abnormal mitochondria with reduced oxygen consumption and ATP concentration, both of which are required for normal 1 cell function.

Indeed, pancreatic Clec16a is required for normal glucose-stimulated insulin release. Moreover, patients harboring a diabetogenic SNP in the Clec16a gene have reduced islet Clec16a expression and reduced insulin secretion. Thus, Clec16a controls p cell function and prevents diabetes by controlling mitophagy. This pathway could be targeted for prevention and control of diabetes and may extend to the pathogenesis of other Clec16a- and Parkin-associated diseases.

It is clear from the foregoing, that therapeutic agents which specifically target Clec16a should have efficacy for the treatment of a variety of disorders, including diabetes, obesity and certain neurological disorders.

### SUMMARY OF THE INVENTION

In accordance with the present invention, a method of management for obesity in a subject in need thereof comprising administering to said subject a therapeutic agent in amount effective to partially reduce CLEC16A expression, thereby managing or reducing obesity is disclosed. In certain embodiments, the therapeutic agent modulates signaling mediated via the CLEC16A gene product. In other embodiments, the therapeutic agent is selected from the group consisting of a small molecule, an antibody, a protein, an oligonucleotide, or an siRNA molecule. The therapeutic agent can also be one or more of an autophagy inhibitor, an inhibitor of the Jak-Stat pathway or an mTor inhibitor.

In some embodiments, the agent is delivered to a cell selected from an adipose cell or an insulin-producing beta cell. In other embodiments, the agent modulates natural killer cell activity.

In other embodiments, the therapeutic agent comprises at least one siRNA molecule provided in Table 1. The therapeutic agent can be targeted to adipose cells, natural killer cells for example. In certain embodiments, the therapeutic agent modulates signaling in an insulin-producing beta cell.

In another aspect, the invention provides a pharmaceutical composition comprising a therapeutic agent which partially inhibits CLEC16A expression in a target cell. In certain embodiments, an siRNA composition comprising at least one nucleotide sequence selected from the group listed in Table 1 in a pharmaceutically acceptable carrier for delivery to a patient. Thus, another aspect of the invention entails a method of partially inhibiting the expression of CLEC16A in a patient comprising administering to said patient at least one siRNA molecule that directs cleavage of a target CLEC16A mRNA sequence present in said patient. Such siRNAs can be used alone or in combination with other siRNAs or agents conventionally used for the management of obesity. Suitable agents, include, without limitation, autophagy inhibitors, mTor inhibitors and Jak-stat pathway inhibitors.

### BRIEF DESCRIPTION OF THE DRAWINGS

FIG. 1. Percent body weight from initial body weight over time. Mice were weighed 3 times per week during the study.

FIG. 2. Tamoxifen-inducible UBC-Cre-Clec16a^{loxP} male and female mice exhibit complete loss of body and visceral fat.

FIG. 3. Food Intake (per gram of body weight in UBC-Cre-Clec16a^{loxP} KO mice as compared to controls.

FIG. 4. Phosphorylation of HSL at Ser563, Ser659 and Ser660 in control and Clec16a KO mice adipose tissue.

FIG. 5. The mRNA expression of lipid catabolism genes (Cpt1b, Ppara), adipogenic genes (Pparg and Adipoq) and thermogenic genes (Ucp1 and Cidea) from gWAT of control and KO mice determined by qPCR (n=8).

FIG. 6. Lipid analysis (cholesterol, triglyceride and free fatty acid) from Clec16a KO vs. control mice serum.

FIG. 7a. Clec16a KO mice exhibit decreased Adiponectin, leptin and LDL-R in comparison to control. FIG. 7b. UBC-Cre-Clec16a^{loxP} KO mice exhibit increased cytokines, chemokines, growth factors and other soluble proteins in adipose tissue compared to control. FIG. 7c. UBC-Cre-Clec16a^{loxP} KO mice exhibit increased cytokines, chemokines, growth factors and other soluble proteins in adipose tissue compared to control. FIG. 7d. UBC-Cre-Clec16a^{loxP} KO mice exhibit increased cytokines/chemokines in plasma compared to control.

FIG. 8. High constitutive expression of IL-16 precursor in Clec16a KO splenocytes and release of bioactive IL-16 by active caspase-3.

FIG. 9. Decreased expression of SOCS1 and SOCS3 in Clec16a KO splenocytes.

FIG. 10A-10C. Tofacitinib, pan JAK/STAT inhibitor, partially rescues the lipodystrophic phenotype and improves survival of UBC-Cre-Clec16a^{loxP} KO mice.

FIG. 11. Predominant Th-1 Cytokine/chemokine in Clec16a KO and rescue with Tofacitinib. The representative graph is quantification of cytokines and chemokine from plasma of Control (Vehicle), KO and KO+Tofacitinib inhibitor treated mice using the Mouse Cytokine Array panel.

FIG. 12. ANA-9 line Immunoblot assay. Lane 1 positive control is showing all the antigens. Lane 2 &3 are probed with sera from control mice; lane 4-8 are probed with sera of Clec16a KO mice.

FIG. 13. Serum Immunoglobulin Isotyping. ELISA was performed to evaluate changes in serum Immunoglobulins isotypes, isotypes and IgG subclasses with control mice and Clec16a KO mice sera (n=10).

FIG. 14A-14C. Clec16a knockout induces disability in mice. KO mice exhibit abnormal neurons in the dorsal root ganglia due to dysregulated mitophagy. Activated microglia with inflammation in the spinal cord dorsal columns and loss of cerebellar Purkinje cells is evident.

### DETAILED DESCRIPTION OF THE INVENTION

In view of the prominent role of CLEC16A SNP associations in variety of autoimmune disorders, we generated a novel whole-body Clec16a inducible knockdown (KD) mouse, in which with tamoxifen treatment CLEC16A expression could be turned off in all organs at desired time points (UBC-Cre-Clec16a^{loxP}). In our study, we discovered that turning off Clec16a in 8-10 week-old mice leads to severe weight loss (˜20%) accompanied by a systemic inflammatory response. Complete KO resulted in additional autoimmune and neurologic phenotypes, including decreased numbers of Schwann cells, the cells which insulate (myelinate) individual nerve fibers (axons); abnormal axons; and myelin debris. In addition, we found pathological changes in Dorsal Root Ganglion (DRG) neurons with accumulated vacuoles and abnormal (swollen or dying) mitochondria—all in concordance with previously published discovery by us, that CLEC16A controls mitophagy and absence of this protein leads to accumulation of unhealthy mitochondria in pancreatic beta cells (Soleimanpour et al., 2014). However, partial knock down of the gene resulted in weight loss without evidence for additional phenotypes, which let us consider CLEC16A (or its pathway) as a target for weight reduction therapy.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The phrase “Type 1 diabetes (TID)” refers to a chronic (lifelong) disease that occurs when the pancreas produces too little insulin to regulate blood sugar levels appropriately. TID, often called juvenile or insulin-dependent diabetes results from altered metabolism of carbohydrates (including sugars such as glucose), proteins, and fats. In type 1 diabetes, the beta cells of the pancreas produce little or no insulin, the hormone that allows glucose to enter body cells. Once glucose enters a cell, it is used as fuel. Without adequate insulin, glucose builds up in the bloodstream instead of going into the cells. The body is unable to use this glucose for energy despite high levels in the bloodstream, leading to increased hunger. In addition, the high levels of glucose in the blood cause the patient to urinate more, which in turn causes excessive thirst. Within 5 to 10 years after diagnosis, the insulin-producing beta cells of the pancreas are completely destroyed, and no more insulin is produced.

An “siRNA” refers to a molecule involved in the RNA interference process for a sequence-specific post-transcriptional gene silencing or gene knockdown by providing small interfering RNAs (siRNAs) that has homology with the sequence of the targeted gene. Small interfering RNAs (siRNAs) can be synthesized in vitro or generated by ribonuclease Ill cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif, USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting CLEC 16A mRNA may be between 15-35 nucleotides in length, and more typically about 21 nucleotides in length. A list of candidate siRNAs directed to CLEC 16A are provided in Table 1.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

Many techniques are available to those skilled in the art to facilitate transformation, transfection, or transduction of the expression construct into a prokaryotic or eukaryotic organism. The terms “transformation”, “transfection”, and “transduction” refer to methods of inserting a nucleic acid and/or expression construct into a cell or host organism. These methods involve a variety of techniques, such as treating the cells with high concentrations of salt, an electric field, or detergent, to render the host cell outer membrane or wall permeable to nucleic acid molecules of interest, microinjection, peptide-tethering, PEG-fusion, and the like.

The term “promoter element” describes a nucleotide sequence that is incorporated into a vector that, once inside an appropriate cell, can facilitate transcription factor and/or polymerase binding and subsequent transcription of portions of the vector DNA into mRNA. In one embodiment, the promoter element of the present invention precedes the 5′ end of the TID specific marker nucleic acid molecule such that the latter is transcribed into mRNA. Host cell machinery then translates mRNA into a polypeptide.

Those skilled in the art will recognize that a nucleic acid vector can contain nucleic acid elements other than the promoter element and the TID specific marker gene nucleic acid molecule. These other nucleic acid elements include, but are not limited to, origins of replication, ribosomal binding sites, nucleic acid sequences encoding drug resistance enzymes or amino acid metabolic enzymes, and nucleic acid sequences encoding secretion signals, localization signals, or signals useful for polypeptide purification.

A “replicon” is any genetic element, for example, a plasmid, cosmid, bacmid, plastid, phage or virus that is capable of replication largely under its own control. A replicon may be either RNA or DNA and may be single or double stranded.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by colorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

Terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organism.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, small molecules, antibodies, peptides, peptide/DNA complexes, and any nucleic acid based molecule, for example an oligo, which exhibits the capacity to modulate the activity of the CLEC16A encoding nucleic acids described herein or the encoded protein. Agents can be evaluated for potential biological activity by inclusion in screening assays described herein below.

The term “modulate” as used herein refers increasing or decreasing. For example, the term modulate refers to the ability of a compound or test agent to interfere with signaling or activity of a gene or protein of the present invention. Therefore, modulating the signaling mediated by CLEC16A means that an agent or compound inhibits or enhances the activity of the protein encoded by the gene. This includes altering lipolysis activity, mitophagy, the activity of natural killer cells, and rates of autoimmune beta cell destruction.

### Pharmaceutical Agents and Therapies

The elucidation of the role played by CLEC16A described herein in cellular metabolism facilitates the development of pharmaceutical compositions useful for treatment and diagnosis of obesity and certain neurological disorders. For example, CLEC16A plays an important role in autophagy. Accordingly, autophagy inhibitors have utility in the present invention. These include, without limitation, SP600125, U0126, 3-Methyladenine, Bafilomycin A1, Chloroquine, LY294002, SB202190, SB203580, SC79 and wortmannin which may act to rescue loss of CLEC16A function in affected individuals.

Jak-Stat inhibitors can also be used to advantage to partially inhibit CLEC16A. Such inhibitors include without limitation,

Ruxolitinib (INCB018424) is the first potent, selective, JAK1/2 inhibitor to enter the clinic with IC50 of 3.3 nM/2.8 nM in cell-free assays, >130-fold selectivity for JAK1/2 versus JAK3. Science, 2018, 10(436) NAT MATER, 2017, 10.1038/NMAT5024 Nat Med, 2015, 10.1038/nm.4013

Tofacitinib citrate (CP-690550 citrate) is a novel inhibitor of JAK with IC50 of 1 nM, 20 nM and 112 nM against JAK3, JAK2, and JAK1, respectively. Cancer Discov, 2012, 2(7):591-7 Nat Cell Biol, 2015, 17(1):57-67 Blood, 2014, 124(5):761-70

AZD1480 is a novel ATP-competitive JAK2 inhibitor with IC50 of 0.26 nM in a cell-free assay, selectivity against JAK3 and Tyk2, and to a smaller extent against JAK1. Phase 1. Nat Cell Biol, 2015, 17(1):57-67 Blood, 2014, 123(10):1516-24 Leukemia, 2012, 26(4):708-15

Fedratinib (SAR302503, TG101348) is a selective inhibitor of JAK2 with IC50 of 3 nM in cell-free assays, 35- and 334-fold more selective for JAK2 versus JAK1 and JAK3. Phase 2. Cell, 2015, 162(2):441-51 Blood, 2014, 123(20):3175-84 J Thorac Oncol, 2016, 11(1):62-71

AT9283 is a potent JAK2/3 inhibitor with IC50 of 1.2 nM/1.1 nM in cell-free assays; also potent to Aurora A/B, Abl(T315I). Phase 2. Cell Stem Cell, 2012, 11(2):179-94 Cancer Res, 2013, 73(20):6310-22 Cancer Lett, 2013, 341(2):224-30 Ruxolitinib Phosphate is the phosphate salt form of ruxolitinib, an orally bioavailable Janus-associated kinase (JAK) inhibitor with potential antineoplastic and immunomodulating activities.

Itacitinib (INCB39110) is an orally bioavailable inhibitor of Janus-associated kinase 1 (JAK1) with potential antineoplastic activity.

PF-06651600 is a potent and irreversible JAK3-selective inhibitor with an IC50 of 33.1 nM but without activity (IC50>10 000 nM) against JAK1, JAK2, and TYK2.

FM-381 is a JAK3 specific reversible covalent inhibitor with IC50 of 127 pM for JAK3 and demonstrates 400-, 2,700- and 3,600-fold selectivity over JAK1, JAK2, and TYK2, respectively.

Momelotinib (CYT387) is an ATP-competitive inhibitor of JAK1/JAK2 with IC50 of 11 nM/18 nM, ˜10-fold selectivity versus JAK3. Phase 3. Nat Cell Biol, 2015, 17(1):57-67 Blood, 2012, 120(19):4093-103 J Clin Invest, 2014, 124(12):5263-74

Tofacitinib (CP-690550, Tasocitinib) is a novel inhibitor of JAK3 with IC50 of 1 nM in cell-free assays, 20- to 100-fold less potent against JAK2 and JAK1. Blood, 2014, 124(5):761-70 Blood, 2012, 120(4):709-19 Blood, 2011, 118(14):3911-21

WP1066 is a novel inhibitor of JAK2 and STAT3 with IC50 of 2.30 μM and 2.43 μM in HEL cells; shows activity to JAK2, STAT3, STATS, and ERK1/2 not JAK1 and JAK3. Phase 1. Int J Cancer, 2014, 135(2):282-94 Exp Neurol, 2015, 271:445-56 J Biol Chem, 2013, 288(36):26167-76

TG101209 is a selective JAK2 inhibitor with IC50 of 6 nM, less potent to Flt3 and RET with IC50 of 25 nM and 17 nM in cell-free assays, ˜30-fold selective for JAK2 than JAK3, sensitive to JAK2V617F and MPLW515L/K mutations. Leukemia, 2014, 28(7):1519-28 Cancer Lett, 2013, 341(2):224-30 ACS Chem Biol, 2014, 9(5):1160-71

Gandotinib (LY2784544) is a potent JAK2 inhibitor with IC50 of 3 nM, effective in JAK2V617F, 8- and 20-fold selective versus JAK1 and JAK3. Phase 2. Cancer Lett, 2013, 341(2):224-30 Gastric Cancer, 2016, 19(1):53-62 Eur J Pharmacol, 2015, 765:188-97

NVP-BSK805 2HCl is a potent and selective ATP-competitive JAK2 inhibitor with IC50 of 0.5 nM, >20-fold selectivity towards JAK1, JAK3 and TYK2. Cancer Lett, 2013, 341(2):224-PLoS One, 2013, 8(5):e63301

Baricitinib (LY3009104, INCB028050) is a selective JAK1 and JAK2 inhibitor with IC50 of 5.9 nM and 5.7 nM in cell-free assays, ˜70 and ˜10-fold selective versus JAK3 and Tyk2, no inhibition to c-Met and Chk2. Phase 3. Nat Cell Biol, 2014, 17(1):57-67 Br J Haematol, 2017, 177(2):271-282

AZ 960 is a novel ATP competitive JAK2 inhibitor with IC50 and Ki of <3 nM and 0.45 nM, 3-fold selectivity of AZ960 for JAK2 over JAK3. Cancer Lett, 2013, 341(2):224-30

CEP33779 is a selective JAK2 inhibitor with IC50 of 1.8 nM, >40- and >800-fold versus JAK1 and TYK2. Biochem Pharmacol, 2014, 91(2):144-56 Biosci Rep, 2017, 37(4)

Pacritinib (SB1518) is a potent and selective inhibitor of Janus Kinase 2 (JAK2) and Fms-Like Tyrosine Kinase-3 (FLT3) with IC50s of 23 and 22 nM in cell-free assays, respectively. Phase 3.

WHI-P154 is a potent JAK3 inhibitor with IC50 of 1.8 μM, no activity against JAK1 or JAK2, also inhibits EGFR, Src, Abl, VEGFR and MAPK, prevents Stat3, but not Stat5 phosphorylation. Oncol Rep, 2017, 37(1):66-76

XL019 is a potent and selective JAK2 inhibitor with IC50 of 2.2 nM, exhibiting>50-fold selectivity over JAK1, JAK3 and TYK2. Phase 1.

S-Ruxolitinib is the chirality of INCB018424, which is the first potent, selective, JAK1/2 inhibitor to enter the clinic with IC50 of 3.3 nM/2.8 nM, >130-fold selectivity for JAK1/2 versus JAK3. Phase 3. Clin Cancer Res, 2015, 21(16):3740-9 Blood Cancer J, 2017, 7(6):e572 Sci Rep, 2016, 6:28473

ZM 39923 HCl is an JAK1/3 inhibitor with pIC50 of 4.4/7.1, almost no activity to JAK2 and modestly potent to EGFR; also found to be sensitive to transglutaminase.

Peficitinib (ASP015K, JNJ-54781532) is an orally bioavailable JAK inhibitor. Phase 3.

Filgotinib (GLPG0634) is a selective JAK1 inhibitor with IC50 of 10 nM, 28 nM, 810 nM, and 116 nM for JAK 1, JAK2, JAK3, and TYK2, respectively. Phase 2.

Decernotinib (VX-509) is a potent and selective JAK3 inhibitor with Ki of 2.5 nM, >4-fold selectivity over JAK1, JAK2, and TYK2, respectively. Phase 2/3.

BMS-911543 is a potent and selective inhibitor of JAK2 with IC50 of 1.1 nM, ˜350-, 75- and 65-fold selective to JAK1, JAK3 and TYK2, respectively. Phase 1/2.

FLLL32 is a potent JAK2/STAT3 inhibitor with IC50 of <5 μM. Cancer Sci, 2016, 107(7):944-54 Eur Rev Med Pharmacol Sci, 2017, 21(13):3005-3011

Curcumol is a pure monomer isolated from Rhizoma Curcumaeis with antitumor activities.

GLPG0634 analogue is a selective JAK1 inhibitor with IC50 of 10 nM, 28 nM, 810 nM, and 116 nM for JAK1, JAK2, JAK3, and TYK2, respectively. Phase 2.

Oclacitinib (PF 03394197) is a novel inhibitor of JAK family members with IC50 ranging from 10 to 99 nM and JAK1-dependent cytokines with IC50 ranging from 36 to 249 nM. It does not inhibit a panel of 38 non-JAK kinases.

Cerdulatinib (PRT-062070) is an oral active, multi-targeted tyrosine kinase inhibitor with IC50 of 12 nM/6 nM/8 nM/0.5 nM and 32 nM for JAK1/JAK2/JAK3/TYK2 and Syk, respectively. Also inhibits 19 other tested kinases with IC50 less than 200 nM. J Immunol, 2016, 197(7):2948-57

Go6976 is a potent PKC inhibitor with IC50 of 7.9 nM, 2.3 nM, and 6.2 nM for PKC (Rat brain), PKCα, and PKCβ1, respectively. Also a potent inhibitor of JAK2 and Flt3. Cell Signal, 2016, 28(9):1422-31 Infect Immun, 2017, e00087-17.

mTor inhibitors also have utility in the methods disclosed herein and include, for example,

Dactolisib (BEZ235, NVP-BEZ235) is a dual ATP-competitive PI3K and mTOR inhibitor for p110α/γ/δ/β and mTOR (p70S6K) with IC50 of 4 nM/5 nM/7 nM/75 nM/6 nM in cell-free assays, respectively. Inhibits ATR with IC50 of 21 nM in 3T3TopBP1-ER cell. Nature, 2017, 728-732 Nature, 2012, 487(7408):505-9 Nat Med, 2015, 10.1038/nm.3855

Rapamycin (Sirolimus) is a specific mTOR inhibitor with IC50 of ˜0.1 nM HEK293 cells. Nature, 2016, 539(7629):437-442 Nat Genet, 2014, 46(4):364-70 Cancer Cell, 2011, 19(6):792-804

Everolimus (RAD001) is an mTOR inhibitor of FKBP12 with IC50 of 1.6-2.4 nM in a cell-free assay. Nat Med, 2015, 10.1038/nm.3855 Cell, 2016, 164(1-2):293-309 Cell, 2016, 164(1-2):293-309

AZD8055 is a novel ATP-competitive mTOR inhibitor with IC50 of 0.8 nM in MDA-MB-468 cells with excellent selectivity (˜1,000-fold) against PI3K isoforms and ATM/DNA-PK. Phase 1. Nat Med, 2015, 10.1038/nm.3855 Cancer Cell, 2015, 27(1):97-108 Cancer Cell, 2015, 27(4):533-46

Temsirolimus (CCI-779, NSC 683864) is a specific mTOR inhibitor with IC50 of 1.76 μM in a cell-free assay. Autophagy, 2011, 7(2):176-87 Cancer Res, 2014, 74(14):3947-58 Mol Cancer, 2014, 13(1):159

PI-103

PI-103 is a multi-targeted PI3K inhibitor for p110α/β/δ/γ with IC50 of 2 nM/3 nM/3 nM/15 nM in cell-free assays, less potent to mTOR/DNA-PK with IC50 of 30 nM/23 nM. Cell, 2013, 153(4):840-54 Leukemia, 2013, 27(3):650-60 Leukemia, 2012, 26(5):927-33

KU-0063794 is a potent and highly specific dual-mTOR inhibitor of mTORC1 and mTORC2 with IC50 of ˜10 nM in cell-free assays, no effect on PI3Ks. Cell Stem Cell, 2012, 10(2):210-7 Circ Res, 2010, 107(10):1265-74 Oncogene, 2013, 10.1038/onc.2013.509

Torkinib (PP242) is a selective mTOR inhibitor with IC50 of 8 nM in cell-free assays; targets both mTOR complexes with >10- and 100-fold selectivity for mTOR than PI3Kδ or PI3Kα/β/γ, respectively. Science, 2016, 353(6302):929-32 Nat Chem Biol, 2013, 9(11):708-14 J Clin Invest, 2015, 10.1172/JC178018

Tacrolimus (FK506) is a 23-membered macrolide lactone, it reduces peptidyl-prolyl isomerase activity in T cells by binding to the immunophilin FKBP12 (FK506 binding protein) creating a new complex. Biochim Biophys Acta, 2015, 1853(10 Pt A):2684-96 Biochim Biophys Acta, 2012, 1833(3):652-62 Biomed Pharmacother, 2013, 67(6):469-73

Ridaforolimus (Deforolimus, MK-8669) is a selective mTOR inhibitor with IC50 of 0.2 nM in HT-1080 cell line; while not classified as a prodrug, mTOR inhibition and FKBP12 binding is similar to rapamycin. Phase 3. Gynecol Oncol, 2016, 141(3):570-9 J Lipid Res, 2014, 55(5):919-28 Mol Pharmaco, 2013, 84(1):104-13

Sapanisertib (INK 128, MLN0128) is a potent and selective mTOR inhibitor with IC50 of 1 nM in cell-free assays; >200-fold less potent to class I PI3K isoforms, superior in blocking mTORC1/2 and sensitive to pro-invasion genes (vs Rapamycin). Phase 1. Cancer Discov, 2014, 4(5):554-63 Cell Rep, 2015, 11(3):446-59 Cell Communication and Signaling, 2015, 13:15

Voxtalisib (SAR245409, XL765) Analogue is a dual inhibitor of mTOR/PI3K, mostly for p110γ with IC50 of 9 nM; also inhibits DNA-PK and mTOR. Phase 1/2. Cell Rep, 2015, 11(3):446-59 Mol Cancer Res, 2014, 12(5):703-13 Endocrinology, 2013, 154(3):1247-59

Torin 1 is a potent inhibitor of mTORC1/2 with IC50 of 2 nM/10 nM in cell-free assays; exhibits 1000-fold selectivity for mTOR than PI3K. Cancer Discov, 2016, 6(7):727-39 Elife, 2015, 4 Am J Pathol, 2014, 184(1):214-29

Omipalisib (GSK2126458, GSK458) is a highly selective and potent inhibitor of p110α/β/δ/γ, mTORC1/2 with Ki of 0.019 nM/0.13 nM/0.024 nM/0.06 nM and 0.18 nM/0.3 nM in cell-free assays, respectively. Phase 1. Proc Natl Acad Sci USA, 2013, 110(10):4015-20 Neuro Oncol, 2016, 18(4):528-37 Mol Cancer Ther, 2015, 14(2):429-39

OSI-027 is a selective and potent dual inhibitor of mTORC1 and mTORC2 with IC50 of 22 nM and 65 nM in cell-free assays, and more than 100-fold selectivity observed for mTOR than PI3Kα, PI3Kβ, PI3Kγ or DNA-PK. Phase 1. Cell Rep, 2015, 11(3):446-59 Eur J Cancer, 2013, 74:41-9 Br J Cancer, 2016, 114(6):650-8

PF-04691502 is an ATP-competitive PI3K(α/β/δ/γ)/mTOR dual inhibitor with Ki of 1.8 nM/2.1 nM/1.6 nM/1.9 nM and 16 nM in cell-free assays, little activity against either Vps34, AKT, PDK1, p70S6K, MEK, ERK, p38, or JNK. Phase 2. Blood, 2015, 10.1182/blood-2014-11-610329 Clin Cancer Res, 2016, 10.1158/1078-0432.CCR-16-1971 Cell Rep, 2015, 11(3):446-59

Apitolisib (GDC-0980, RG7422) is a potent, class I PI3K inhibitor for PI3Kα/β/δ/γ with IC50 of 5 nM/27 nM/7 nM/14 nM in cell-free assays, respectively. Also a mTOR inhibitor with Ki of 17 nM in a cell-free assay, and highly selective versus other PIKK family kinases. Phase 2. Cancer Discov, 2014, 4(5):554-63 Breast Cancer Res, 2014, 16(4):406 Mol Cancer Ther, 2015, 14(8):1928-38

GSK1059615 is a dual inhibitor of PI3Kα/β/δ/γ (reversible) and mTOR with IC50 of 0.4 nM/0.6 nM/2 nM/5 nM and 12 nM, respectively. Phase 1. Nature, 2012, 486(7404):532-6 Nat Chem Biol, 2013, 9(11):708-14 Exp Mol Med, 2015, 47:e143 Gedatolisib (PF-05212384, PKI-587) is a highly potent dual inhibitor of PI3Kα, PI3Kγ and mTOR with IC50 of 0.4 nM, 5.4 nM and 1.6 nM in cell-free assays, respectively. Phase 2. Cell Rep, 2015, 11(3):446-59 Pigment Cell Melanoma Res, 2014, 10.1111/pcmr.12268 Mol Cancer Ther, 2015, 14(2):429-39

WYE-354 is a potent, specific and ATP-competitive inhibitor of mTOR with IC50 of 5 nM, blocks mTORC1/P-S6K(T389) and mTORC2/P-AKT(S473) not P-AKT(T308), selective for mTOR than PI3Kα (>100-fold) and PI3Kγ (>500-fold). Cancer Lett, 2015, 359(1):97-106 Exp Mol Med, 2015, 47:e143 Exp Mol Med, 2015, 47:e143

Vistusertib (AZD2014) is a novel mTOR inhibitor with IC50 of 2.8 nM in a cell-free assay; highly selective against multiple PI3K isoforms (α/β/γ/δ). AZD2014 showed no or weak binding to the majority of kinases when tested at 1 μM.

These compositions may comprise, in addition to one of the above substances, a pharmaceutically acceptable excipient, carrier, buffer, stabilizer or other materials well known to those skilled in the art. Such materials should be non-toxic and should not interfere with the efficacy of the active ingredient.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual.

As it is presently understood, RNA interference involves a multi-step process. Double stranded RNAs are cleaved by the endonuclease Dicer to generate nucleotide fragments (siRNA). The siRNA duplex is resolved into 2 single stranded RNAs, one strand being incorporated into a protein-containing complex where it functions as guide RNA to direct cleavage of the target RNA (Schwarz et al, Mol. Cell. 10:537 548 (2002), Zamore et al, Cell 101:25 33 (2000)), thus silencing a specific genetic message (see also Zeng et al, Proc. Natl. Acad. Sci. 100:9779 (2003)).

The invention includes a method of treating CLEC16A related disorders such as obesity and neurological disease in a mammal. An exemplary method entails administering to the mammal a pharmaceutically effective amount of CLEC16A siRNA. The siRNA inhibits the expression of CLEC16A. Preferably, the mammal is a human. The term “patient” as used herein refers to a human.

Specific siRNA preparations directed at inhibiting the expression of CLEC16A, as well as delivery methods are provided as a novel therapy to treat obesity. SiRNA oligonucleotides directed to CLEC16A specifically hybridize with nucleic acids encoding CLEC16A and interfere with CLEC16A gene expression. The siRNA can be delivered to a patient in vivo either systemically or locally with carriers, as discussed below. The level of siRNA expressed can be controlled by methods known to those of skill in the art. The compositions of the invention may be used alone or in combination with other agents or genes encoding proteins to augment the efficacy of the compositions.

A “membrane permeant peptide sequence” refers to a peptide sequence which is able to facilitate penetration and entry of the CLEC16A inhibitor across the cell membrane. Exemplary peptides include without limitation, the signal sequence from Karposi fibroblast growth factor exemplified herein, the HIV tat peptide (Vives et al., J. Biol. Chem., 272:16010-16017, 1997), Nontoxic membrane translocation peptide from protamine (Park et al., FASEB J. 19(11):1555-7, 2005), CHARIOT® delivery reagent (Active Motif; U.S. Pat. No. 6,841,535) and the antimicrobial peptide Buforin 2.

In one embodiment of the invention siRNAs are delivered for therapeutic benefit. There are several ways to administer the siRNA of the invention to in vivo to treat obesity including, but not limited to, naked siRNA delivery, siRNA conjugation and delivery, liposome carrier-mediated delivery, polymer carrier delivery, nanoparticle compositions, plasmid-based methods, and the use of viruses.

siRNA composition of the invention can comprise a delivery vehicle, including liposomes, for administration to a subject, carriers and diluents and their salts, and/or can be present in pharmaceutically acceptable formulations. This can be necessary to allow the siRNA to cross the cell membrane and escape degradation. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192; Beigelman et al., U.S. Pat. No. 6,395,713 and Sullivan et al., PCT WO 94/02595 further describe the general methods for delivery of nucleic acid molecules. These protocols can be utilized for the delivery of virtually any nucleic acid molecule.

The frequency of administration of the siRNA to a patient will also vary depending on several factors including, but not limited to, the type and severity of the obesity or neurological disease to be treated, the route of administration, the age and overall health of the individual, the nature of the siRNA, and the like. It is contemplated that the frequency of administration of the siRNA to the patient may vary from about once every few months to about once a month, to about once a week, to about once per day, to about several times daily.

Pharmaceutical compositions that are useful in the methods of the invention may be administered systemically in parenteral, oral solid and liquid formulations, ophthalmic, suppository, aerosol, topical or other similar formulations. In addition to the appropriate siRNA, these pharmaceutical compositions may contain pharmaceutically-acceptable carriers and other ingredients known to enhance and facilitate drug administration. Thus, such compositions may optionally contain other components, such as adjuvants, e.g., aqueous suspensions of aluminum and magnesium hydroxides, and/or other pharmaceutically acceptable carriers, such as saline. Other possible formulations, such as nanoparticles, liposomes, resealed erythrocytes, and immunologically based systems may also be used to administer the appropriate siRNA to a patient according to the methods of the invention. The use of nanoparticles to deliver siRNAs, as well as cell membrane permeable peptide carriers that can be used are described in Crombez et al., Biochemical Society Transactions v 35:p 44 (2007).

Methods of the invention directed to treating obesity involve the administration of CLEC16A siRNA in a pharmaceutical composition. CLEC16A siRNA is administered to an individual as a pharmaceutical composition comprising CLEC16A siRNA and a pharmaceutically acceptable carrier. Pharmaceutically acceptable carriers are well known in the art and include aqueous solutions such as physiologically buffered saline, other solvents or vehicles such as glycols, glycerol, oils such as olive oil or injectable organic esters.

A pharmaceutically acceptable carrier can contain physiologically acceptable compounds that act, for example, to stabilize the CLEC16A siRNA or increase the absorption of the agent. Such physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the CLEC16A siRNA.

One skilled in the art appreciates that a pharmaceutical composition comprising CLEC16A siRNA can be administered to a subject by various routes including, for example, orally or parenterally, such as intravenously (i.v.), intramuscularly, subcutaneously, intraorbitally, intranasally, intracapsularly, intraperitoneally (i.p.), intracisternally, intra-tracheally (i.t.), or intra-articularly or by passive or facilitated absorption. The same routes of administration can be used other pharmaceutically useful compounds, for example, small molecules, nucleic acid molecules, peptides, antibodies and polypeptides as discussed hereinabove.

A pharmaceutical composition comprising CLEC16A siRNA inhibitor also can be incorporated, if desired, into liposomes, microspheres, microbubbles, or other polymer matrices (Gregoriadis, Liposome Technology, Vols. I to III, 2nd ed., CRC Press, Boca Raton Fla. (1993)). Liposomes, for example, which consist of phospholipids or other lipids, are nontoxic, physiologically acceptable and metabolizable carriers that are relatively simple to make and administer. The pharmaceutical preparation comprises a siRNA targeting CLEC 16A or an expression vector encoding for an siRNA targeting CLEC 16A. Such pharmaceutical preparations can be administered to a patient for treating obesity or neurological diseases associated with aberrant CLEC16A function.

Expression vectors for the expression of siRNA molecules preferably employ a strong promoter which may be constitutive or regulated. Such promoters are well known in the art and include, but are not limited to, RNA polymerase II promoters, the T7 RNA polymerase promoter, and the RNA polymerase III promoters U6 and H1 (see, e.g., Myslinski et al. (2001) Nucl. Acids Res., 29:2502 09).

A formulated siRNA composition can be a composition comprising one or more siRNA molecules or a vector encoding one or more siRNA molecules independently or in combination with a cationic lipid, a neutral lipid, and/or a polyethyleneglycol-diacylglycerol (PEG-DAG) or PEG-cholesterol (PEG-Chol) conjugate. Non-limiting examples of expression vectors are described in Paul et al., 2002, Nature Biotechnology, 19, 505; Miyagishi and Taira, 2002, Nature Biotechnology, 19, 497; Lee et al., 2002, Nature Biotechnology, 19, 500-505.

A lipid nanoparticle composition is a composition comprising one or more biologically active molecules independently or in combination with a cationic lipid, a neutral lipid, and/or a polyethyleneglycol-diacylglycerol (i.e., polyethyleneglycol diacylglycerol (PEG-DAG), PEG-cholesterol, or PEG-DMB) conjugate. In one embodiment, the biologically active molecule is encapsulated in the lipid nanoparticle as a result of the process of providing and aqueous solution comprising a biologically active molecule of the invention (i.e., siRNA), providing an organic solution comprising lipid nanoparticle, mixing the two solutions, incubating the solutions, dilution, ultrafiltration, resulting in concentrations suitable to produce nanoparticle compositions.

Nucleic acid molecules can be administered to cells by incorporation into other vehicles, such as biodegradable polymers, hydrogels, cyclodextrins. (see for example Gonzalez et al., 1999, Bioconjugate Chem., 10, 1068-1074; Wang et al., International PCT publication Nos. WO 03/47518 and WO 03/46185), poly(lactic-co-glycolic) acid (PLGA) and PLCA microspheres (see for example U.S. Pat. No. 6,447,796 and US Patent Application Publication No. US 2002130430), biodegradable nanocapsules, and bioadhesive microspheres, or by proteinaceous vectors (O'Hare and Normand, International PCT Publication No. WO 00/53722).

Cationic lipids and polymers are two classes of non-viral siRNA delivery which can form complexes with negatively charged siRNA. The self-assembly PEG-ylated polycation polyethylenimine (PEI) has also been used to condense and protect siRNAs (Schiffelers et al., 2004, Nuc. Acids Res. 32. 141-110). The siRNA complex can be condensed into a nanoparticle to allow efficient uptake of the siRNA through endocytosis. Also, the nucleic acid-condensing property of protamine has been combined with specific antibodies to deliver siRNAs and can be used in the invention (Song et al., 2005, Nat. Biotech. 23:709-717).

In order to treat an individual having obesity or a neurological disease, to alleviate a sign or symptom of the disease, CLEC16A siRNA should be administered in an effective dose. The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. One skilled in the art would know that the amount of CLEC16A siRNA required to obtain an effective dose in a subject depends on many factors, including the age, weight and general health of the subject, as well as the route of administration and the number of treatments to be administered. In view of these factors, the skilled artisan would adjust the particular dose so as to obtain an effective dose for treating an individual having obesity.

The effective dose of CLEC16A siRNA will depend on the mode of administration, and the weight of the individual being treated. The dosages described herein are generally those for an average adult but can be adjusted for the treatment of children. The dose will generally range from about 0.001 mg to about 1000 mg.

The concentration of CLEC16A siRNA in a particular formulation will depend on the mode and frequency of administration. A given daily dosage can be administered in a single dose or in multiple doses so long as the CLEC16A siRNA concentration in the formulation results in the desired daily dosage. One skilled in the art can adjust the amount of CLEC16A siRNA in the formulation to allow administration of a single dose or in multiple doses that provide the desired concentration of CLEC16A siRNA over a given period of time.

In an individual suffering from obesity, in particular a more severe form of the disease, administration of CLEC16A siRNA can be particularly useful when administered in combination, for example, with a conventional agent for treating such a disease. The skilled artisan would administer CLEC16A siRNA, alone or in combination and would monitor the effectiveness of such treatment using routine methods such as pulmonary function determination, radiologic, immunologic or, where indicated, histopathologic methods.

Administration of the pharmaceutical preparation is preferably in an “effective amount” this being sufficient to show benefit to the individual. This amount prevents, alleviates, abates, or otherwise reduces the severity of obesity symptoms in a patient.

The pharmaceutical preparation is formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

As mentioned previously, a preferred embodiment of the invention comprises delivery of the CLEC16A siRNA to a patient in need thereof, and candidate siRNA compositions for use in the invention are provided in Table 1. The sequences in Table I include several siRNA duplexes (i.e., sense and antisense sequences for a CLEC16A target region), as well as several sequences of ‘sense’ strand alone. Those of skill in the art can determine the sequence of an antisense siRNA strand based on the disclosure of the sense strand, and will appreciate the difference between “U” and “T” designations in the sequences which correspond to RNA and DNA molecules, respectively.

The following example is provided to illustrate certain embodiments of the invention. It is not intended to limit the invention in any way.

### Example I

Given the prominent role of CLEC16A SNP associations in variety of autoimmune disorders, we generated a novel whole-body Clec16a tamoxifen inducible knockout (KO) mouse, where tamoxifen treatment turns off CLEC16A expression in all organs at time points of interest (UBC-Cre-Clec16a^{loxP}). In our study, we discovered that turning off Clec16a in 8-10-week-old mice leads to severe weight loss (˜20%), robust inflammatory response and development of severe neurological symptoms, including ataxia. The mice exhibited a neuronal phenotype including tremors, impaired gait, and dystonic postures that worsen over time. Pathological analysis revealed that degenerating sensory axons, and Purkinje cell loss in the cerebellum account for this phenotype. Activated microglia and astrocytes were found in affected regions of the CNS. Affected and unaffected regions of the CNS and PNS showed increased levels of proteins related to impaired mitophagy and autophagy. These findings suggest that mitophagy and/or autophagy plays a role in certain types of spinocerebellar degeneration. Thus, ubiquitous inducible knockout of Clec16a in mice results in progressive neurodegeneration resembling spinocerebellar ataxia.

Our Clec16a KO mouse model shows mitochondrial defect and accumulation of unhealthy mitochondria. Our lab and others have shown a connection between Clec16a and autophagy in immunological and neurological cells (Redmann et al., 2016; Soleimanpour et al., 2014; Tam et al., 2017). We show that the extreme weight loss observed in the Clec16a KO mice is due to lipolysis (lipophagy) observed by the complete loss of fat and an increased phosphorylation of Hormone sensitive lipase (HSL) proteins in Western blots. Increased food intake observed in KO mice fails to rescue the white adipose atrophy. mRNA expression revealed upregulation of upregulation of catabolic and thermogenic genes together with downregulation of downstream adipogenic genes promoting HSL-mediated lipolysis in adipose tissue. Serum lipid analysis revealed significant decrease in Cholesterol, Triglycerides and free fatty acids and decreased adiponectin, leptin and upregulated LDL-Receptors in adipose tissue. Normal adipose tissue growth and function is critical to maintaining metabolic homeostasis and its excess (e.g. obesity) or absence (e.g. lipodystrophy) is associated with severe metabolic disease. In addition, elevated cytokine levels as measured by Proteome Profiler Mouse XL Cytokine Array, were observed concurrent with the lipolysis and could contribute to further wasting and the progressive neurodegeneration resembling spinocerebellar ataxia observed in the mice. Evidence for a link to SOCS proteins and the JAK/STAT pathway with autoimmune inflammatory phenotype and spinocerebral ataxia are presented respectively. Our whole body inducible Clec16a KO mouse, therefore provides a comprehensive murine model for use in future elucidation of mechanism and dug targets involved in healthy form of weight loss, Autoimmune-inflammatory phenotype and spinocerebellar degeneration.

Clec16a KO of >60% in UBC-Cre-Clec16a^{loxP} mice exhibit adipose tissue atrophy and severe weight loss. We employed UBC-Cre-Clec16a^{loxP} mice—an inducible KO model to study CLEC16A's role in autoimmunity. We choose this model to circumvent possible embryonic lethality and determine the effect of CLEC16A loss in adult mice.

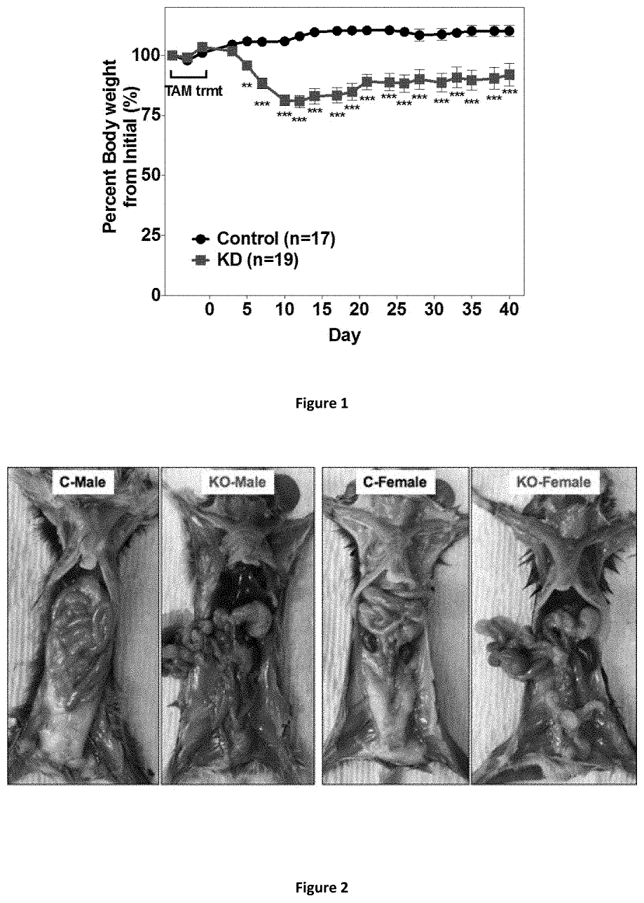

The first visible observation we made in control and Clec16a KO mice, fed on regular chow diet was difference in body weight. Clec16a knockout mice exhibit severe weight loss starting 1 week after initiation of tamoxifen treatment in comparison to control mice. During the same time period, control mice showed a healthy appearance and maintained their body weight throughout the study in comparison to Clec16a KO (FIG. 1). All mice with greater than 60% of Clec16a KO exhibited significant reduction of body weight which deteriorated over the length of the study with increased severity in neurological symptoms, and an elevated morbidity rate (FIG. 1). According to previously reports, gender differences do exist for T1D pathogenesis, MS, and other autoimmune disorders. The disease incidence lays around 60-80% in females and 20-30% in males. For the purpose of healthy examination and to determine the cause of weight loss, the Clec16a KO and control mice were dissected.

Compared to control mice, both male and female Clec16a KO mice exhibited near to complete absence of typical gonadal adipose fat tissue (FIG. 2). Further examination indicated that all the white adipose tissue (WAT) depots, including gonadal, inguinal, mesenteric, retroperitoneal, perineal, and pericardial, were remarkably reduced or absent in Clec16a KO mice (FIG. 2). Thus, Clec16a loss promotes body weight reduction and fat loss leading to lipodystrophy of adipose tissue, a phenotype similar to that observed in mammalian models with lipodystrophy and has profound effect on WAT deposits in adult mice. These results suggest that Clec16a KO modulates lipid metabolism and triggers abnormal fat loss possibly mediated by lipolysis (lipophagy).

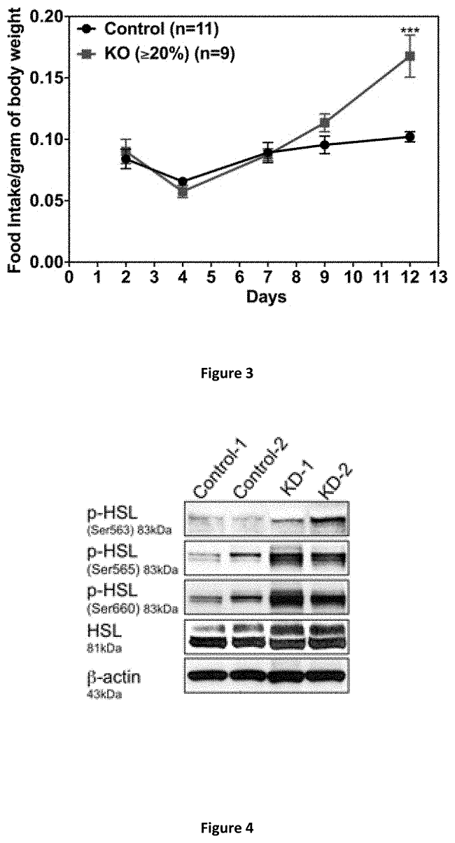

UBC-Cre-Clec16a^{loxP} KO mice exhibit increased food intake. We performed food intake study to rule out the amount of food consumed as a possible reason behind the severe weight loss. Clec16a KO mice consumed as much or more food in comparison to the control mice. Thus, less food consumption is not the reason behind the weight loss of Clec16a KO mice (FIG. 3). Despite increased food consumption Clec16a KO mice kept losing weight rapidly. These results suggest that CLEC16A modulates fat loss either by reduced efficiency in storage or an increased energy expenditure, or both in the Clec16a KO mice.

Clec16a KO leads to abnormal fat loss by accelerated lipolysis in adipose tissues of UBC-Cre-Clec16aloxP KO mice. To examine the signaling underlying the fat loss in Clec16a KO we used immunoblot analysis to assess the role of Clec16a in inducing lipolysis (lipophagy). HSL (hormone-sensitive lipase) is a key enzyme in the mobilization of fatty acids in adipocytes as well as non-adipocytes. Triacylglycerol is stored in lipid droplets as a primary energy reserve. During lipolysis, triacylglycerols in adipocytes are hydrolyzed into free fatty acids and glycerol. Phosphorylation of HSL at Ser563, Ser659, and Ser660 by PKA stimulates HSL activity, which in turn catalyzes the hydrolysis of triacylglycerol. We found increased phosphorylation of HSL (FIG. 4). Our results indicate that CLEC16A functions to restrain lipolysis in adipose tissues and loss of CLEC16A triggers lipolysis.

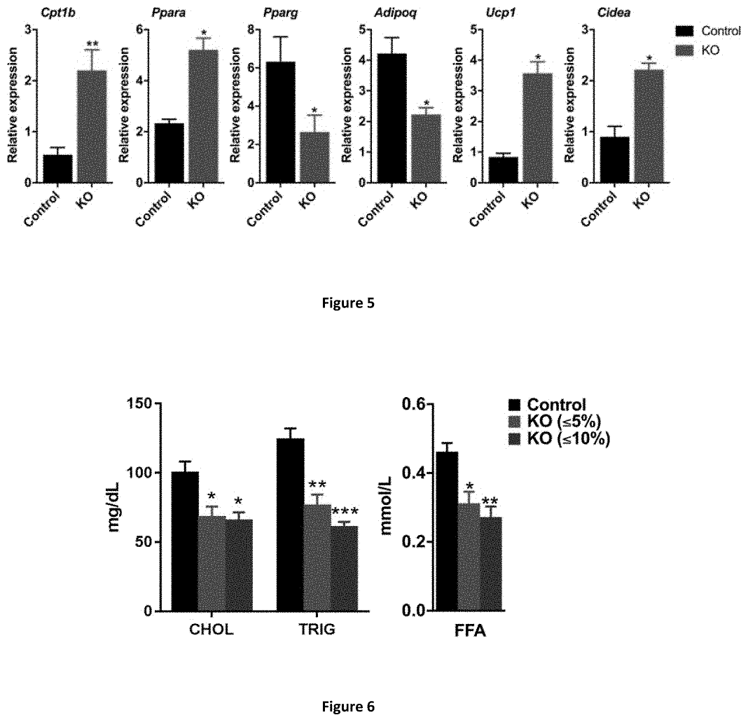

To gain insight in the mechanism(s) whereby Clec16a mediates its effect on energy expenditure to induce fat loss, we measured expression of key genes regulating lipid metabolism in gonadal white adipose tissue (gWAT) of Clec16a KO mice. We found that carnitine palmitoyltransferase 1b (Cpt1b), a gene essential for adipose tissue fatty acid oxidation, was significantly upregulated in Clec16a KO gWAT, along with the upstream transcription factor, peroxisome proliferator-activated receptor alpha (Ppara). Further, the expression of the adipogenic gene, Pparg and its downstream target adiponectin precursor (Adipoq) were significantly reduced in Clec16a KO gWAT. Thermogenic genes, thermogenin (Ucp1) and cell death inducing DFFA-like effector A (Cidea) were significantly upregulated in WAT of Clec16a mice (FIG. 5). Thus, fat loss in Clec16a KO mouse is mediated through upregulation of catabolic and thermogenic genes together with downregulation of downstream adipogenic genes promoting HSL-mediated lipolysis. Increased food intake in KO mice fails to rescue the white adipose atrophy.

Lipolysis is defined as the catabolism of triacylglycerols stored in cellular lipid droplets. New findings that lipolytic products and intermediates participate in cellular signaling processes and is particularly important in many non-adipose tissues unveils a previously underappreciated aspect of lipolysis, which may be relevant for human diseases. Normal adipose tissue growth and function is critical to maintaining metabolic homeostasis and its excess (e.g. obesity) or absence (e.g. lipodystrophy) is associated with severe metabolic disease. Decreased triglyceride storage leads to adipocyte lipotoxicity, mitochondrial dysfunction and increased oxidative stress. This results in production of inflammatory mediators and deregulated release of free fatty acids. This contributes to impaired insulin sensitivity and adverted liver, muscles and heart functions leading to early complications.

Serum lipid analysis of Clec16a KO and control mice. We also performed lipid analysis on serum of control and Clec16a KO (score 2 and 4) mice. A significant decrease in cholesterol, triglycerides and free fatty acid was observed in the serum of Clec16a KO mice compared to control (FIG. 6). These results suggest that, controlled Clec16a expression could be of therapeutic potential in promoting a healthier form of weight loss.

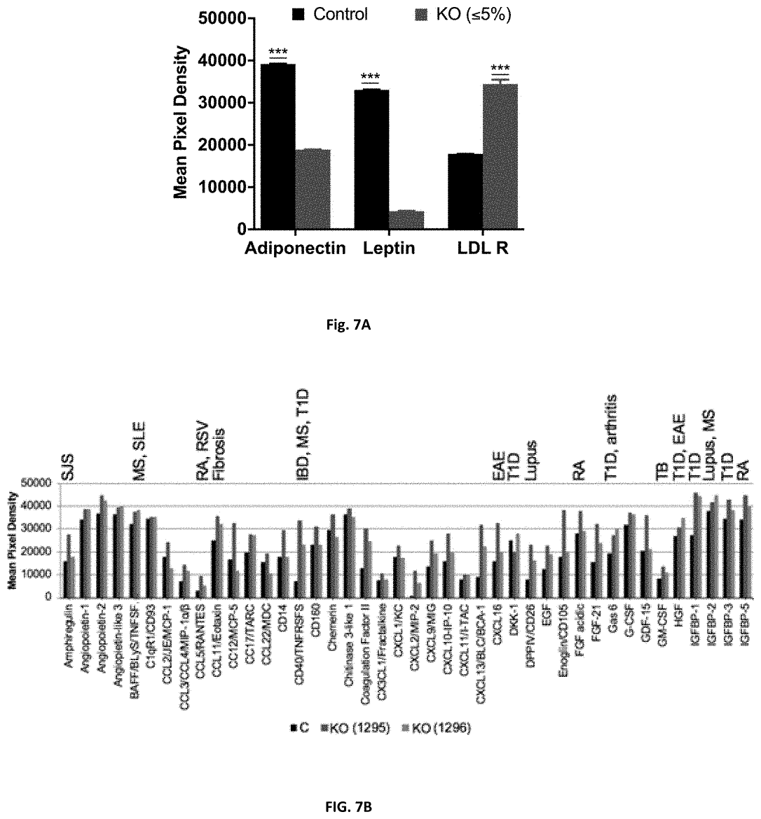

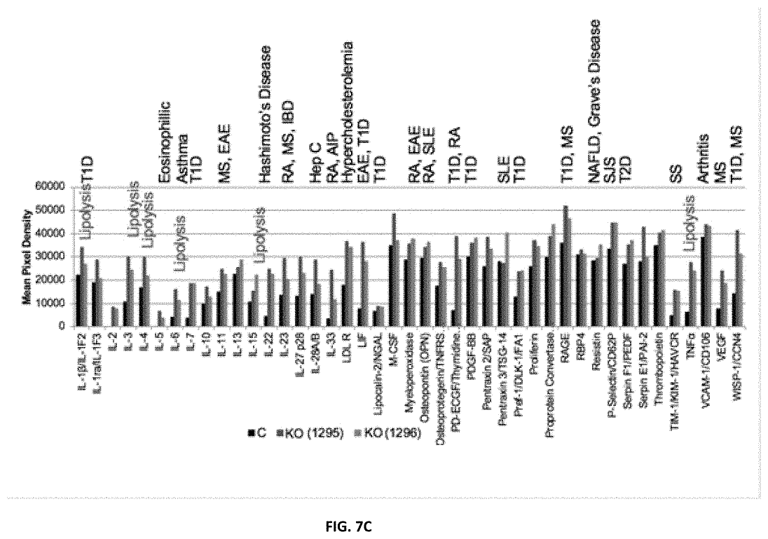

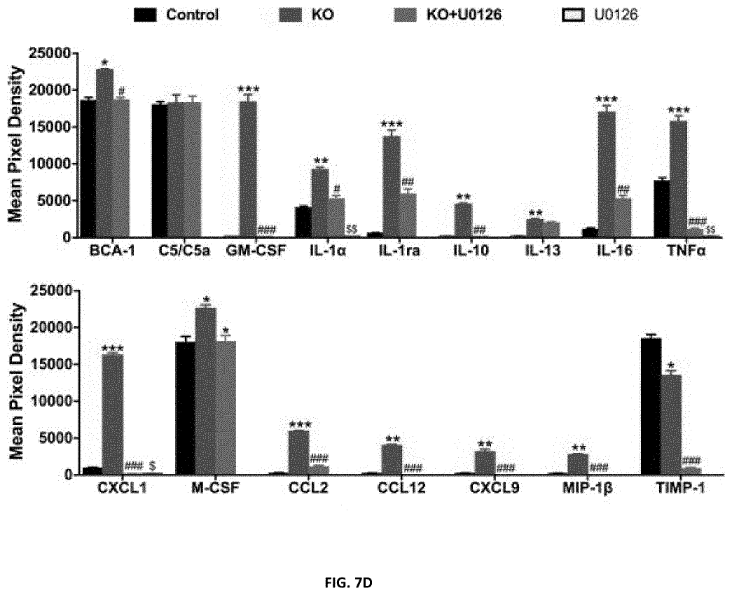

UBC-Cre-ER^{T2}-Clec16a^{loxP} KO mice exhibit increased cytokine levels in adipose tissue and plasma. To gain insight how Clec16a KO, weight loss and lipolysis promote a dynamic immune response in murine adipose tissue and may contribute to disease pathogenesis, we evaluated adipose tissue and plasma of control and Clec16a KO mice in the Proteome Profiler Mouse XL Cytokine Array. The Proteome Profiler Mouse XL Cytokine Array Kit is a membrane-based sandwich immunoassay allows parallel determination of the relative levels of selected mouse cytokines and chemokines. Adiponectin and leptin from adipose tissue play a key role in energy homeostasis and metabolism. Clec16a KO mice exhibit decreased adiponectin, leptin and LDL-R compared to control (FIG. 7a). In addition, we saw up-regulation of several cytokine, chemokines and inflammatory markers in adipose tissue of Clec16a KO mice that have been associated with several autoimmune disorders (FIGS. 7b, c). Our results indicate that the signal originates from dysregulated lipolysis (adipose tissue) and promotes wasting.

To gain insight in the inflammatory mechanism involved in the development, progression and pathogenesis of various autoimmune diseases, we profiled plasma from control, KO and U0126-treated KO mice for cytokines and chemokine using Mouse Cytokine Array panel. Plasma from Clec16a KO mice showed upregulation of Th1 cytokines (TNF-α, IL-1, & IL-16), vs. low levels of Th2 (IL-10 & IL-13) and elevated levels key chemokines GM-CSF, KC (CXCL1) JE (MCP-1), MCP-5, MIG (CXCL9), MIP-1b (CCL4) in comparison to control (FIG. 7d).

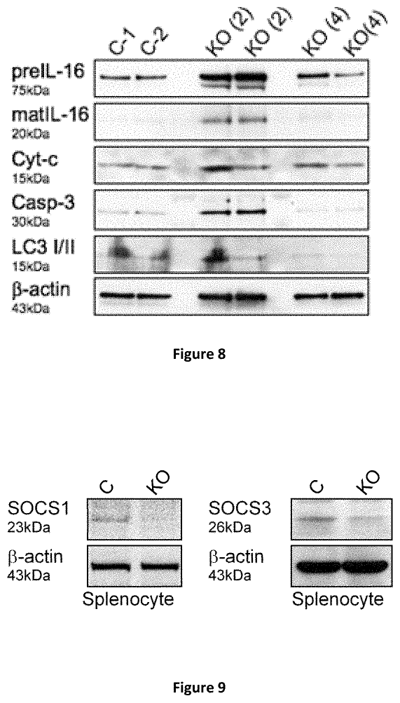

U0126 inhibitor treatment reversed all the up regulated cytokines and chemokines, suggesting that the inflammatory mechanism involved with autoimmune risk is mediated by dysregulated mitophagy and can be corrected by mitophagy inhibitors. Our results provide critical evidence in support for role of dysregulated lipolysis and Clec16a loss leads to progression of autoimmunity as depicted in graph (FIG. 7b-d). Increase in IL-16 in the plasma cytokine levels could contribute to the neurological degeneration seen in the Clec16a KO mice (FIG. 7d). Our Immunoblot analysis indicates constitutively high expression of IL-16 precursor and bioactive IL-16 in Clec16a KO splenocyte further support the role of cytokine mediated neurodegeneration (FIG. 8). The cytokine IL-16 is a CD4⁺T cell-specific chemoattractant that is biased towards CD4+ Th1 cells. IL-16 precursor is constitutively expressed in lymphocytes and during CD4⁺ T cell activation; active caspase-3 cleaves and releases C-terminal bioactive IL-16. The connection between increased cytokines and neurodegeneration is known (Khaibullin et al., 2017). It is also known that the presence of CLEC16A MS risk alleles correlate with reduced SOCS1 and DEXI expression in the thymus through a regulatory element (Leikfoss et al., 2013).

SOCS Protein Expression is Decreased in UBC-Cre-Clec16a^{loxP} KO Mice.

Based on the observed loss of visceral and subcutaneous fat, and food intake study, our ubiquitous Clec16a KO mice display a phenotype similar to that observed in lipodystrophy. Dysregulated lipolysis contributes to lipotoxicity, mitochondrial dysfunction and increased oxidative stress resulting in production of inflammatory mediators. CLEC16's genomic location next to the suppressor of cytokine signaling 1 (SOCS1) gene and the expression specificity in immune cells including dendritic cells, B & T-lymphocytes and natural killer (NK) cells, which are pivotal in the pathogenesis of several autoimmune disorders, led us to hypothesize that CLEC16A exerts its effect on a wide variety of immune cells via modulating SOCS expression and regulating cytokine signaling. The SOCS (suppressor or cytokine signaling) family members are negative regulators of cytokine signal transduction that inhibit the Jak/Stat pathway. These proteins are important regulators of cytokine signaling, proliferation, differentiation, and immune responses and are involved in regulating over 30 cytokines, including interleukins, growth hormone (GH), interferon, leptin, and leukemia inhibitory factor. SOCS1 shares the most homology with SOCS3 and both are highly induced by cytokines. Both SOCS1 and SOCS3 directly inhibit Jak activity. Jak (Janus Kinase) and Stat (signal transducer and activator of transcription) proteins are play important roles in inflammatory immune responses (Fenner et al., 2006), and therefore, regulation of Jak/Stat signaling is crucial to prevent aberrant signaling which can lead to disease progression.

To examine the mechanism involved behind the inflammatory cytokine storm, we examined the levels of SOCS1 and SOCS3 expression in an immunoblot analysis. Splenocytes from Clec16a KO exhibit decreased expression of SOCS1 and SOCS3 compared to control (FIG. 9). With SOCS protein levels decreased, cytokine production is not suppressed resulting in the increased cytokine levels contributing to inflammation, concomitant lipolysis and neurodegeneration observed in the Clec16a KO mice. In light of our data, and given the association of Clec16a with several autoimmune disorders, it appears that the molecular link between CLEC16A, lipophagy, and SOCS is abnormal and leads to autoimmune disorders.

The Pan JAK Inhibitor Tofacitinib Suppresses SOCS1-JAK-STAT Mediated Cytokine Signaling and Improves Survival of Clec16a KO Mice.

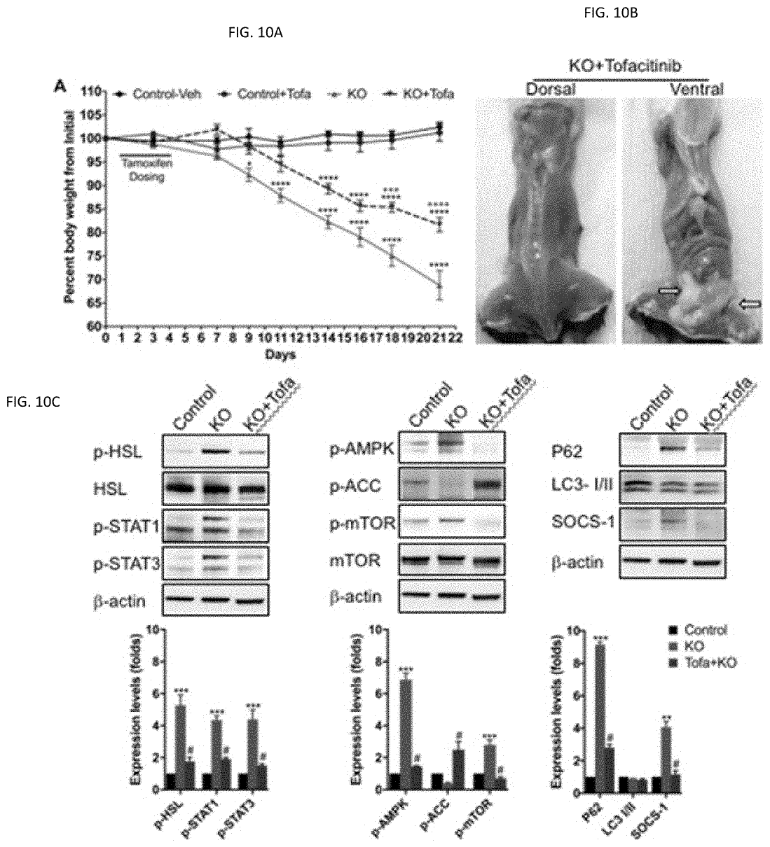

In light of the above findings and established CLEC16A association with several autoimmune disorders, we hypothesized that upregulated JAK/STAT signaling observed in CLEC16A KO mice could be rescued using a JAK/STAT inhibitor. Tofacitinib treatment significantly rescued the fat and weight loss in CLEC16A KO mice and improved the survival curve (FIG. 10A-10C).

To examine the signaling mechanism underlying the rescue, we performed immunoblot analysis on gWAT isolated from control, KO and Tofacitinib treated mice. We evaluated HSL, STATs, AMPK, mTOR, P62, LC3I/II and SOCS-1 expression (FIG. 10C). Tofacitinib treatment significantly reversed the SOCS1 expression observed in CLEC16A KO. Immunoblot analysis revealed significant upregulation of phospho-HSL in CLEC16AKO and significant reversal of p-HSL comparable to normal in CLEC16A KO+Tofacitinib mice. CLEC16A KO mice adipose further exhibits significant upregulation of p-STAT1 and p-STAT3. Tofacitinib rescued the inflammatory phenotype by downregulating the p-STAT1 and p-STAT3 to normal levels comparable to control. We observed significantly enhanced phosphorylation of AMPK. However, its target ACC exhibited reduced phosphorylation in CLEC16A KO. Tofacitnib treatment significantly reduced the upregulated p-AMPK and promoted the phosphorylation of ACC. Another downstream effector of AMPK is mTOR signaling that controls a plethora of functions and is a regulator of autophagy. We observed significant increase in phosphorylation of mTOR and reversal in tofacitinib treated CLEC16A KO mice correcting the autophagy defect. Over-activation of mTOR indicates inhibition of autophagy/lipophagy as evident by significant accumulation of P62 in CLEC16A KO mice. Tofacitinib treatment also rescues the autophagy/lipophagy defect (FIG. 10C). Thus, Tofacitinib exerts its multifaceted effect on AMPK, mTOR, HSL, STATs and autophagy/lipophagy signaling and rescues the lipodystrophic inflammatory phenotype exhibited by CLEC16A KO mice.

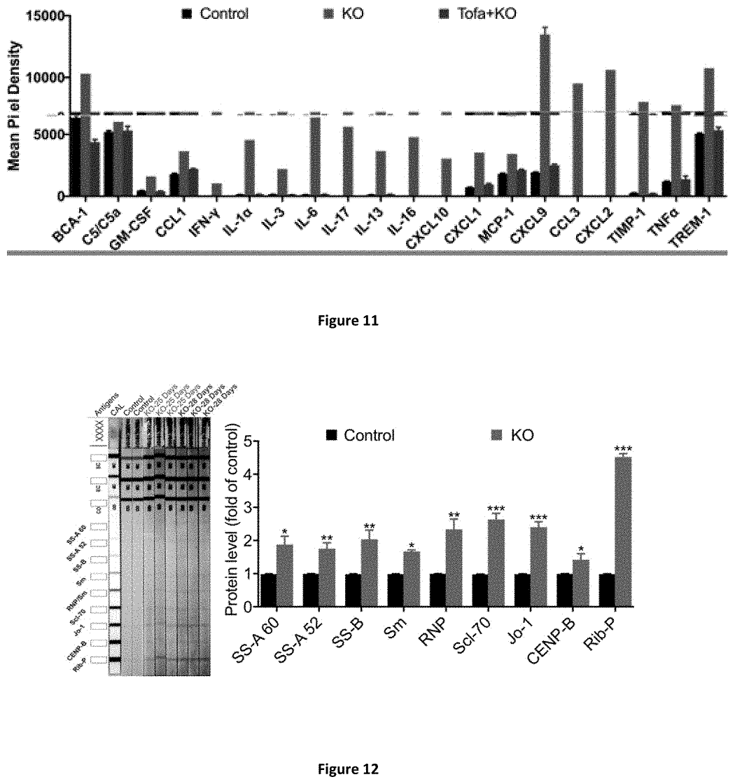

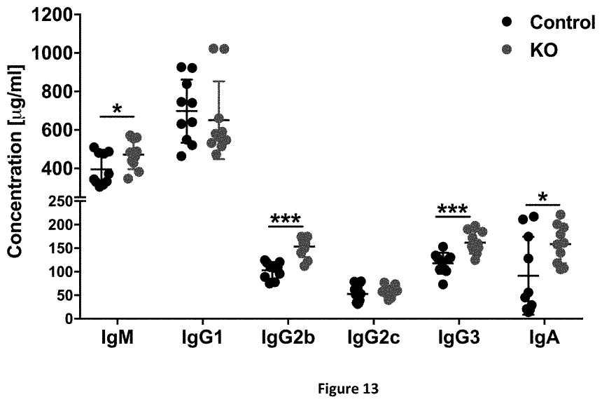

Increased cytokines/chemokine levels reflect upon the inflammatory mechanism utilized during the development, progression and pathogenesis of various autoimmune and inflammatory diseases. Our results indicate that CLEC16A knockout inflammatory phenotype is attenuated by Tofacitinib (FIG. 11).

CLEC16A KO induces susceptibility to Autoimmunity in mice. We used C57Bl/6 mice, to test the hypothesis that altered CLEC16A expression can induce autoimmune responses in a genetic background that does not spontaneously express an autoimmune phenotype (Hudson et al., 2003). This model can therefore be used not only to trace the pathogenesis of the autoimmune responses, but also to explore how CLEC16A KO might trigger the autoimmune response through modified immune regulation.

CLEC16A KO-induced Autoantibodies. Serum samples from Control and CLEC16A KO mice were assayed for antibodies to various nuclear antigens using a line assay Western blot. ANA-9-Line Immunoblot assay is a membrane-based enzyme immunoassay for the semi-quantitative measurement of IgG class autoantibodies to extractable nuclear antigens SS-A 52, SS-A 60, SS-B, RNP/Sm, Sm, centromere B, Jo-1, Scl-70 and ribosomal P proteins in serum or plasma. These results show that CLEC16A KO led to production of antinuclear antibodies indicative of systemic autoimmune disease.

Our finding of upregulated specific antibodies in the sera from CLEC16A KO mice is interesting as these antibodies are also found in SLE and other systemic autoimmune diseases. Further characterization of the specific target autoantibodies in this model is needed, as this may provide clues regarding the mechanisms of lost tolerance to self-antigens.

Serum Immunoglobulin Isotyping. In order to determine whether CLEC16A KO led to changes in serum immunoglobulin isotypes, isotypes and the IgG subclasses were measured sera. IgM and IgA showed significant upregulation in CLEC16A KO mice sera compared to control. IgG subclasses IgG1, IgG2b, and IgG3 were statistically significant. IgG2c showed no change. IgG2b, the predominant subclass in sera of C57Bl6 mice showed upregulation in CLEC16A KO mice. This antibody subclass actively binds complement and therefore can be considered potentially pathogenic leading to Th1 phenotype. Another IgG subclass IgG3, potent proinflammatory antibody despite its shorter half-life and effective in induction of effector functions showed significant upregulation in CLEC16A KO. Serum Immunoglobulin Isotyping results are indicate of excessive inflammatory response.

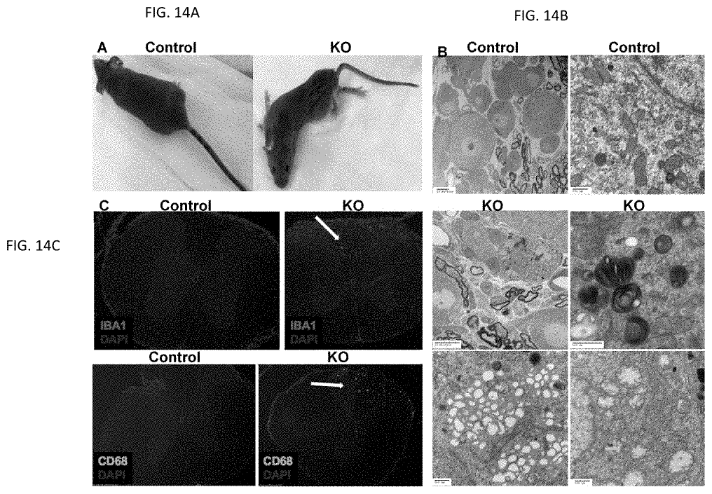

Ubiquitous inducible knockout of CLEC16A in mice results in progressive neurodegeneration resembling spinocerebellar ataxia. Our whole body inducible CLEC16A KO mice exhibits a neuronal phenotype including tremors, impaired gait, and dystonic postures that worsen overtime (FIG. 14A-14C).

Pathological analysis revealed that degenerating sensory axons, and Purkinje cell loss in the cerebellum account for this phenotype. Activated microglia and astrocytes were found in affected regions of the CNS. Affected and unaffected regions of the CNS and PNS showed increased levels of proteins related to mitophagy and autophagy. These findings suggest that mitophagy and/or autophagy might play a role in some kinds of spinocerebellar degeneration. The selective involvement of cerebellar and primary sensory neurons models a human disease known as spinocerebellar ataxia, which has diverse genetic causes (Huang and Verbeek, 2018).

Our whole body inducible Clec16a KO mouse, therefore provides a comprehensive murine model for use in future elucidation of mechanism and dug targets involved in healthy form of weight loss, Autoimmune-inflammatory phenotype and spinocerebellar ataxia.

### Conclusion

Our results underscore critical role of CLEC16A action in immune cells and indicate that a delicate balance of CLEC16A activity appears to be needed for cellular homeostasis. In our study, we discovered that turning off CLEC16A in 8-10-week-old mice leads to severe weight loss (˜20%), robust inflammatory response and development of severe neurological symptoms, including ataxia with progressive neurodegeneration resembling spinocerebellar ataxia. In, patient populations harboring variants that result in CLEC16A hypofunction, drugs with modulatory effects on mitophagy/autophagy/SOCS1 signaling could compensate for the attenuated CLEC16A activity and present formidable candidates for targeted interventions.

### Example II

### siRNA Compositions and Methods for Partial Inhibition of CLEC16A Expression

Based on these data, partial blockage of CLEC16A (or any of its pathway members) should result in weight loss which will benefit patients with obesity, without impacting susceptibility for autoimmune or neurological conditions.

As discussed above, one way to achieve partial reduction of CLEC16A expression levels is to introduce CLEC16A directed siRNAs into cells. A series of RNAs targeting CLEC16A are provided herein below.

TABLE 1
Candidate siRNA constructs for CLEC16A
Corresponding GenBank Accession
number: NM_ 015226

| | |
|---|---|
| SEQ ID NO: 1 | CAAAAAAAUGUCUUCUCCCtt |
| SEQ ID NO: 2 | AAAUAUCCAAAGGAGAUGUtt |
| SEQ ID NO: 3 | CUGCUAUAGCAGAAAACCAtt |
| SEQ ID NO: 4 | UUCUCUUUUAUUGCCAAGUtt |
| SEQ ID NO: 5 | GAGGUUUUCUAACCCUCGGtt |
| SEQ ID NO: 6 | CAAAACGUGGUACAGAUACtt |
| SEQ ID NO: 7 | UUCUGUGACUGUGGUGUUUtt |
| SEQ ID NO: 8 | CUAGCAGGUUCCGGUUCUGtt |
| SEQ ID NO: 9 | CUCCACUAGCAGGUUCCGGtt |
| SEQ ID NO: 10 | GAUGGUCUCCACUAGCAGGtt |
| SEQ ID NO: 11 | CAAGAAGAAAACAAACAUAtt |
| SEQ ID NO: 12 | ACACGUAACGGCCCGACUUtt |
| SEQ ID NO: 13 | UGAGGUCUCGUGACUGAUGtt |
| SEQ ID NO: 14 | CUCAUCAGAAAAGUCAAAUtt |
| SEQ ID NO: 15 | GAGUUUUAACGAAAGUGUU~~ |
| SEQ ID NO: 16 | AAAGAAAUGGACAGUGUGGtt |
| SEQ ID NO: 17 | UGUGUACAGGGCAAAGUCAtt |
| SEQ ID NO: 18 | GGUUGAAAAACUUGAUGGCtt |
| SEQ ID NO: 19 | UUCAGGGUGGUUGAAAAACtt |
| SEQ ID NO: 20 | ACUUUAUAGACAUUCAAAGtt |
| SEQ ID NO: 21 | CCCAAUGAACCAGACCAAAtt |
| SEQ ID NO: 22 | UCUGCACGCAGUCAUCGAGtt |
| SEQ ID NO: 23 | GUUGAUGAUCAGGAUGUCAtt |
| SEQ ID NO: 24 | AUCGUUGAGGAACUCACAGtt |
| SEQ ID NO: 25 | GUGGUCAGUGAGCACAUCGtt |
| SEQ ID NO: 26 | AGCGGUGCAUGAUGUAUAAtt |
| SEQ ID NO: 27 | AAUGACUUCAGCUAACGAGtt |
| SEQ ID NO: 28 | GAUCACCAUUCAGAAUGACtt |
| SEQ ID NO: 29 | CAUCUCAGACAGAUCACCAtt |
| SEQ ID NO: 30 | CUGAAUAUCCUGUUCAGUCtt |
| SEQ ID NO: 31 | AACUUCUCUGAAUAUCCUGtt |
| SEQ ID NO: 32 | CCUCUUGCCCUUGUGCUUGtt |
| SEQ ID NO: 33 | UGGGCCCUUUCUCCUCAUCtt |
| SEQ ID NO: 34 | GGCAUCCUCGGUGGGCCCUtt |
| SEQ ID NO: 35 | GCUGGAUUCGCUCUAAUUUtt |
| SEQ ID NO: 36 | AUCUGGCUGGGCAGCGUUGtt |
| SEQ ID NO: 37 | CAGCGUCGCCAGCCGGAUCtt |
| SEQ ID NO: 38 | ACUCAUCAGGACUUGCUGCtt |
| SEQ ID NO: 39 | AGCCAGCACUCAUCAGGACtt |
| SEQ ID NO: 40 | GCAGGCCAGGUGCACGuCCtt |
| SEQ ID NO: 41 | ACAUGUCCAAAAAAAUGUC~~ |
| SEQ ID NO: 42 | UCAUGCUCCUAUACUCAUCtt |
| SEQ ID NO: 43 | AGGCUUGUGGAUGGUGAUGtt |
| SEQ ID NO: 44 | CUGGUCAGGAAGGUGAGGCtt |
| SEQ ID NO: 45 | UGCGCUCCUGGCCACGUUCtt |
| SEQ ID NO: 46 | GGCUGCGCUCCUGGCCACGtt |
| SEQ ID NO: 47 | UGGGUGCUCCCAGUGUGUCtt |
| SEQ ID NO: 48 | GGAGCGCAGGGAUAGGUGGtt |
| SEQ ID NO: 49 | UGAUGUUUUUGGACUUCUUtt |
| SEQ ID NO: 50 | GUUGAUGUUUUUGGACUUCtt |
| SEQ ID NO: 51 | AGUGUUGAUGUUUUUGGACtt |
| SEQ ID NO: 52 | AACCUUAGUGUUGAUGUUUtt |
| SEQ ID NO: 53 | CAAACCUUAGUGUUGAUGUtt |
| SEQ ID NO: 54 | ACUUCAUUAUUCCACAGGCtt |
| SEQ ID NO: 55 | UUUCCAGGAAGGUACUUCAtt |
| SEQ ID NO: 56 | GUAUCUCCCUUGUUUUUGU~~ |
| SEQ ID NO: 57 | GGAAGCUGUCUCUGUUUGGtt |
| SEQ ID NO: 58 | UCUUCACGGUCCCAACUGGtt |
| SEQ ID NO: 59 | GGCCACAGGGUCGGGAGUCtt |
| SEQ ID NO: 60 | GUCUGAUGCAUCUGGGUCCtt |
| SEQ ID NO: 61 | CCGAGUAGAAAGGAACAUCtt |
| SEQ ID NO: 62 | UGGCAGGUCUGGAGGAAAGtt |
| SEQ ID NO: 63 | GGAGACAGAGACCAUGUUCtt |
| SEQ ID NO: 64 | CGAGGAGACAGAGACCAUGtt |
| SEQ ID NO: 65 | AAACUGCCCUCGGCAGGCCtt |
| SEQ ID NO: 66 | UUGGCAGGACUGUGUUUCCtt |
| SEQ ID NO: 67 | CUCCUUGGCAGGACUGUGU~~ |
| SEQ ID NO: 68 | UGGGCGCCACUCCCCCUCCtt |
| SEQ ID NO: 69 | CCUUUCUGAGCUGUGCGUU~~ |
| SEQ ID NO: 70 | GUUUGUCCUUCCUUUGGGUtt |
| SEQ ID NO: 71 | CUUGGGGUGGGGCAGCCACtt |
| SEQ ID NO: 72 | CUGGGUGAGGUGGGGUCCUtt |
| SEQ ID NO: 73 | ACAAAAAUUAUUUACAUAUtt |
| SEQ ID NO: 74 | UCACUGGGACAAAAAUUAUtt |
| SEQ ID NO: 75 | GUUCUCACUGGGACAAAAAtt |
| SEQ ID NO: 76 | GCUCAGAGGCAUCGAGGUUtt |
| SEQ ID NO: 77 | AGGCUCAGAGGCAUCGAGGtt |
| SEQ ID NO: 78 | UCACUCAGUUUACCCCGAtt |
| SEQ ID NO: 79 | GGAGGAACCGCGAGCCGCCtt |
| SEQ ID NO: 80 | GUGGAUGUUGCGGGAAGUCtt |
| SEQ ID NO: 81 | GUGGUCCAAGGAGUGGAUGtt |
| SEQ ID NO: 82 | UGUUCUGUGACUGUGGUGUtt |
| SEQ ID NO: 83 | CAAAUACAGAGCUGUCAUUtt |
| SEQ ID NO: 84 | GUCAAAUACAGAGCUGUCAtt |
| SEQ ID NO: 85 | GAAGAAAACAAACAUAUUCtt |
| SEQ ID NO: 86 | CGACUUUUGCCGCAAGAUGtt |
| SEQ ID NO: 87 | GCACACGUAACGGCCCGACtt |
| SEQ ID NO: 88 | GAUGUUCUCAAAGAGGAUGtt |
| SEQ ID NO: 89 | AUAGAAUUUACGUAGUUAUtt |
| SEQ ID NO: 90 | GAUGAUAGAAUUUACGUAG~~ |
| SEQ ID NO: 91 | UUAUGAACGAUGAUAGAAUtt |
| SEQ ID NO: 92 | UUGAGUUUUAACGAAAGUG~~ |
| SEQ ID NO: 93 | GGACAGUGUGGUUGUUGAGtt |
| SEQ ID NO: 94 | GAAAUGGACAGUGUGGUUGtt |
| SEQ ID NO: 95 | AAAGUCAUUGGUGUGCUCAtt |
| SEQ ID NO: 96 | AACCAUGCUUUCAGGGUGG~ |
| SEQ ID NO: 97 | CAGCAAUUCUAACCAUGCUtt |
| SEQ ID NO: 98 | GUUAUGGUUCUUACAGCAAtt |
| SEQ ID NO: 99 | ACAUUCAAAGUUAUGGUUCtt |
| SEQ ID NO: 100 | UAGACAUUCAAAGUUAUGGtt |
| SEQ ID NO: 101 | CAAUGACACUUUAUAGACAtt |
| SEQ ID NO: 102 | CUGGUUAUCCAAUGACACUtt |
| SEQ ID NO: 103 | GUAGUGCAGCAUGGCCUGG~~ |
| SEQ ID NO: 104 | GAAGUAAGGAACAGCAGUUtt |
| SEQ ID NO: 105 | GAGAAGUAAGGAACAGCAGtt |
| SEQ ID NO: 106 | UGCCACCAGAUCACUCAGUtt |
| SEQ ID NO: 107 | GAGGGGCAGGAAGAGCCUGtt |
| SEQ ID NO: 108 | UUCUCCUCCCUUGUCCUGGtt |
| SEQ ID NO: 109 | UUUCGGCCGUUCUCCUCCCtt |
| SEQ ID NO: 110 | GCAGGCUAAUUUUCGGCCG~~ |
| SEQ ID NO: 111 | AGACACCGGCAGGCUAAUU~~ |
| SEQ ID NO: 112 | AGAGACACCGGCAGGCUAAtt |
| SEQ ID NO: 113 | AUGCUGGGCUUGGCAGAACtt |
| SEQ ID NO: 114 | GAAGCACCGAAUGCUGGGCtt |
| SEQ ID NO: 115 | CUCGAGUGUCUCGGUGGGUtt |
| SEQ ID NO: 116 | CCGCCUCUUGCCCUUGUGCtt |
| SEQ ID NO: 117 | UUGCACCCGCCUCUUGCCCtt |
| SEQ ID NO: 118 | UCUCUUUUGCACCCGCCUCtt |
| SEQ ID NO: 119 | UUUUGUAGUUGGGUCUCUUtt |
| SEQ ID NO: 120 | GUUUUUGUAGUUGGGUCUCtt |
| SEQ ID NO: 121 | UUCCCCAACGUUUUUGUAGtt |
| SEQ ID NO: 122 | UUCUUCUUCCCCAACGUUUtt |
| SEQ ID NO: 123 | UCUUCUUCUUCCCCAACGUtt |
| SEQ ID NO: 124 | CUUUCUCCUCAUCUUCUUCtt |
| SEQ ID NO: 125 | GCCCUUUCUCCUCAUCUUCtt |
| SEQ ID NO: 126 | UAGCCUUCUCGGCGUCUUCtt |
| SEQ ID NO: 127 | CUUUAGCCUUCUCGGCGUCtt |
| SEQ ID NO: 128 | ACCCUCUGUACCUUUAGCCtt |
| SEQ ID NO: 129 | UGAACCACCCUCUGUACCUtt |
| SEQ ID NO: 130 | CCACUCGUCUUGAUGCCUUtt |
| SEQ ID NO: 131 | CCCCACUCGUCUUGAUGCCtt |
| SEQ ID NO: 132 | UUCACUCUCCCCACUCGUCtt |
| SEQ ID NO: 133 | UGAUCACCAUCUCGAUCUCtt |
| SEQ ID NO: 134 | GGCGGCCAGCUCUGAGAGCtt |
| SEQ ID NO: 135 | UUUCUCCUCGUCCGUGGUGtt |
| SEQ ID NO: 136 | GCAGGUGGCGGCGGCGCUUtt |
| SEQ ID NO: 137 | GAGCAGGUGGCGGCGGCGCtt |
| SEQ ID NO: 138 | CCAGGAAGGGUCUGCUCCAtt |
| SEQ ID NO: 139 | UUCAGGAUCCAUGCCUUUAtt |
| SEQ ID NO: 140 | UUUUUCAGGAUCCAUGCCU~~ |
| SEQ ID NO: 141 | GAGCUGGAUUCGCUCUAAUtt |
| SEQ ID NO: 142 | UUUGGCACGGGGAGCUGGAtt |
| SEQ ID NO: 143 | GUGGUCUUCUCGGCCGCAUtt |
| SEQ ID NO: 144 | CGGGUGGUUGUAGGUGGUCtt |
| SEQ ID NO: 145 | UCUUUCAGCUAGCGGGUGGtt |
| SEQ ID NO: 146 | UCAUGAUCCUGAUGAGUCUtt |
| SEQ ID NO: 147 | GUACAAGGUGAACACUUUCtt |
| SEQ ID NO: 148 | GUCGUACAAGGUGAACACU~ |
| SEQ ID NO: 149 | UCCACGUUCAUGGGCUUCAtt |
| SEQ ID NO: 150 | AUAUUCCACGUUCAUGGGCtt |
| SEQ ID NO: 151 | CAUCAUGAGAUAUUCCACGtt |
| SEQ ID NO: 152 | AGGCGUCCAUCAUGAGAUA~~ |
| SEQ ID NO: 153 | CCCGUCAGUGGCGUGCCUGtt |
| SEQ ID NO: 154 | AUCGCCACACGGCAGCCGCtt |
| SEQ ID NO: 155 | CCGGAUGGCCCGCCGGGUCtt |
| SEQ ID NO: 156 | UCUCAGGCUCCCCUCGCAAtt |
| SEQ ID NO: 157 | AUCCAGGACAUCAUCAGUCtt |
| SEQ ID NO: 158 | UGCAAUCAAGUCGCUGUUAtt |
| SEQ ID NO: 159 | ACAUGCAAUCAAGUCGCUGtt |
| SEQ ID NO: 160 | CUGGACCAUGCCGCCAUCCtt |
| SEQ ID NO: 161 | CUGCAAUAGGCCUGCAAACtt |
| SEQ ID NO: 162 | CUGGAGGAUGGGGAAGGGCtt |
| SEQ ID NO: 163 | GCCUUUGGCCAGGCGCUGCtt |
| SEQ ID NO: 164 | CCUUGCCUGGAUGCGGCCUtt |
| SEQ ID NO: 165 | CUCUGCAUCUUCAUGCGCCtt |
| SEQ ID NO: 166 | GGCAGCUAUUCUCUGCAUCtt |
| SEQ ID NO: 167 | AGGUCCAGGAGGGCAGCUAtt |
| SEQ ID NO: 168 | ACUUCAGUGGUGGGCUGGAtt |
| SEQ ID NO: 169 | CGAGUCCAAACCCCAGGACtt |
| SEQ ID NO: 170 | CACGGCGAAGCCUGGCACCtt |
| SEQ ID NO: 171 | GACGGGGAGCUGUGCUGGU~~ |
| SEQ ID NO: 172 | GAAGGUGAGGCUUAGGCAGtt |
| SEQ ID NO: 173 | GCUUCCGUUUCGUUGACGAtt |
| SEQ ID NO: 174 | AGAGUCUGCUUCCGUUUCGtt |
| SEQ ID NO: 175 | GCUUAGAGUCUGCUUCCGUtt |
| SEQ ID NO: 176 | UGCUGGGCUUAGAGUCUGCtt |
| SEQ ID NO: 177 | GGCCACGUUCUUGCUGGGCtt |
| SEQ ID NO: 178 | GGGGGACAAGGGUCAGCGAtt |
| SEQ ID NO: 179 | GCUCAGCACAUGCAGCCUCtt |
| SEQ ID NO: 180 | AACGUGGGGCUUCUUCCCAtt |
| SEQ ID NO: 181 | UUCAAGGACAACGUGGGGCtt |
| SEQ ID NO: 182 | AUGCAAAGUGAAAAAGGAAtt |
| SEQ ID NO: 183 | GCCGGUGCAGUCAUCUGCAtt |
| SEQ ID NO: 184 | CAGUGACCUGGUCCAAUUCtt |
| SEQ ID NO: 185 | AGUCUGCUUUUCUACAAAUtt |
| SEQ ID NO: 186 | UGUUUAUCUAAGUCUGCUUtt |
| SEQ ID NO: 187 | GAUGUUUAUCUAAGUCUGCtt |
| SEQ ID NO: 188 | GAAGUUUUAAAAAUAAAUGtt |
| SEQ ID NO: 189 | CUUCUUUUAAAUAGAAGUUtt |
| SEQ ID NO: 190 | GACUUCUUUUAAAUAGAAGtt |
| SEQ ID NO: 191 | AUGACAUCAAACCUUAGUGtt |
| SEQ ID NO: 192 | UUUCACAUGACAUCAAACCtt |
| SEQ ID NO: 193 | AACUGUUAUUAUUACACUUtt |
| SEQ ID NO: 194 | UUAACUGUUAUUAUUACACtt |
| SEQ ID NO: 195 | GAAAUCUUAACUGUUAUUAtt |
| SEQ ID NO: 196 | CAUGAAAUCUUAACUGUUAtt |
| SEQ ID NO: 197 | GAUCAUGAAAUCUUAACUGtt |
| SEQ ID NO: 198 | UGAAAAUGAUCAUGAAAUC~~ |
| SEQ ID NO: 199 | GUGAGUAACAAAGAAUCACtt |
| SEQ ID NO: 200 | AUUCCACAGGCUUGCAGAGtt |
| SEQ ID NO: 201 | CCAGGAAGGUACUUCAUUAtt |
| SEQ ID NO: 202 | AAACUUUCCAGGAAGGUACtt |
| SEQ ID NO: 203 | UUAAAAAAUAAUCCAAACUIt |
| SEQ ID NO: 204 | ACAUGUAUCUCCCUUGUUUtt |
| SEQ ID NO: 205 | AUACAUGUAUCUCCCUUGUtt |
| SEQ ID NO: 206 | GAGAAUACAUGUAUCUCCC~~ |
| SEQ ID NO: 207 | CCUUUUCCUGGAGAAAUCUtt |
| SEQ ID NO: 208 | GCUGGCUACAUUCCUCCUU~~ |
| SEQ ID NO: 209 | GAGCUGGCUACAUUCCUCCtt |
| SEQ ID NO: 210 | GAGUGGGGAGCUGGCUACAtt |
| SEQ ID NO: 211 | AAGCUGUCUCUGUUUGGUUtt |
| SEQ ID NO: 212 | UGCUGGAAGCUGUCUCUGU~~ |
| SEQ ID NO: 213 | GGUCCCAACUGGUUCCUUCtt |
| SEQ ID NO: 214 | CACGGUCCCAACUGGUUCCtt |
| SEQ ID NO: 215 | GAGGGUGUCUUCCUUUGAUtt |
| SEQ ID NO: 216 | ACGUAGAGGGUGUCUUCCUtt |
| SEQ ID NO: 217 | AGGUGACGUAGAGGGUGUCtt |
| SEQ ID NO: 218 | CUCUGGGGACACCAUGCCCtt |
| SEQ ID NO: 219 | GAACCAUGGGGUUUCCAUAtt |
| SEQ ID NO: 220 | GAUGCAUCUGGGUCCUUGCtt |
| SEQ ID NO: 221 | CUGUGUUUCCUUCAUGAGGtt |
| SEQ ID NO: 222 | GCACAGCUACCCAGAGGGC~~ |
| SEQ ID NO: 223 | UCUAUGGAAGCAAAGGUCC~~ |
| SEQ ID NO: 224 | CCCCUUUCUGAGCUGUGCGtt |
| SEQ ID NO: 225 | UCUGCCCAUGUGGCCCCCUtt |
| SEQ ID NO: 226 | GUCGUGGUUUGUCCUUCCUtt |
| SEQ ID NO: 227 | CGGUGGUCGUGGUUUGUCC~~ |
| SEQ ID NO: 228 | UGGCCACGGUGGUCGUGGUtt |
| SEQ ID NO: 22 | CUUCCUUCUCUUCCAGGGAtt |
| SEQ ID NO: 230 | CCACCCUGCCUUCCUUCUCtt |
| SEQ ID NO: 231 | CCGCUCCACCCUGCCUUCCtt |
| SEQ ID NO: 232 | CCCCCCGCUCCACCCUGCCtt |
| SEQ ID NO: 233 | CCUUCUCUCCAUGAUGGUCtt |
| SEQ ID NO: 234 | UCUCCUGAUGCUGUGGUCCtt |
| SEQ ID NO: 235 | CCCUCUUGCUCUUAAAAAAtt |
| SEQ ID NO: 236 | CUCUCUACCCCUCUUGCUCtt |
| SEQ ID NO: 237 | UUGAUCCUCUCUACCCCUCtt |
| SEQ ID NO: 238 | AUCUCCAGCCAGGGCCAGC~~ |
| SEQ ID NO: 239 | GCCCCACAGACAGAGUCAUtt |
| SEQ ID NO: 240 | GCCACUUGCCUUCUCUAGUtt |
| SEQ ID NO: 241 | GGUGGGGCAGCCACUUGCCtt |
| SEQ ID NO: 242 | UGUUCCUCCUGGUCACGCCtt |
| SEQ ID NO: 243 | UGGAGUGAGCUGCAGGCUGtt |
| SEQ ID NO: 244 | CCAGUUGGAGCCCAGAGACtt |
| SEQ ID NO: 245 | UCAGUGGUUACAAGACCAGtt |
| SEQ ID NO: 246 | CUCCUUCAGUGCUCAGUGGtt |
| SEQ ID NO: 247 | CUGACCAAGACCUCUCUCCtt |
| SEQ ID NO: 248 | CUGGUCCUCCCAGUCACCUtt |
| SEQ ID NO: 249 | CUGCCCUGAGCAGUGUCUUtt |
| SEQ ID NO: 250 | CCCUGCCCUGAGCAGUGUCtt |
| SEQ ID NO: 251 | CAUGGGGACUGCCCUUUUCtt |
| SEQ ID NO: 252 | CCACAUGGGGACUGCCCUUtt |
| SEQ ID NO: 253 | GCCCACAUGGGGACUGCCC~~ |
| SEQ ID NO: 254 | GGUGUCCCCCAGACGCAAGtt |
| SEQ ID NO: 255 | CUCCUUACAUAAGCAAAGCtt |
| SEQ ID NO: 256 | GGCUGGCUCCCAGACCUCCtt |
| SEQ ID NO: 257 | AGCGCCCCAGCUAUGAGGUtt |
| SEQ ID NO: 258 | CUUCCCACACUCCUGGCUCtt |
| SEQ ID NO: 259 | ACAGCCCCCACUGUGGGCCtt |
| SEQ ID NO: 260 | AAGUGCUCUCUGCAGGGACtt |
| SEQ ID NO: 261 | GCCAGCCCUGCUCCCUGACtt |
| SEQ ID NO: 262 | CAAAGCCAAGGUUUGGGAGtt |
| SEQ ID NO: 263 | CAAUAUUCAAAGCCAAGGUtt |
| SEQ ID NO: 264 | GCACACCUCCACAACAAUA~~ |
| SEQ ID NO: 265 | ACCUGCUGGGACAGGUACCtt |
| SEQ ID NO: 266 | CGAUGGUGAAGGCUGGCCCtt |
| SEQ ID NO: 267 | GUGAACGCAAGUGUCUGGG~~ |
| SEQ ID NO: 268 | CACCUGCCCCUUAGGUUGCtt |
| SEQ ID NO: 269 | UCUUCACCUGCCCCUUAGGtt |
| SEQ ID NO: 270 | GCGUCUGGCAGGGCUGCGCtt |
| SEQ ID NO: 271 | ACGGUGCAUCUCAGAGACCtt |
| SEQ ID NO: 272 | AUCAGUUCACCCCACGCCUtt |
| SEQ ID NO: 273 | ACAAGAAGAUCAAAAUCAGtt |
| SEQ ID NO: 274 | AAUGCUUCAGAUUUAUUUAtt |
| SEQ ID NO: 275 | UUAAAUGCUUCAGAUUUAUtt |
| SEQ ID NO: 276 | UACAUUAAAUGCUUCAGAUtt |
| SEQ ID NO: 277 | AGAUGACUACAUUAAAUGCtt |
| SEQ ID NO: 278 | CAAUGUCAAGAUGACUACAtt |
| SEQ ID NO: 279 | AAAAAUUAUUUACAUAUUUtt |
| SEQ ID NO: 280 | UGCUGGCGAAAGCAGGUACtt |
| SEQ ID NO: 281 | UUUCAUUUCCACCCUCGUGtt |
| SEQ ID NO: 282 | AGGAAGUUCCAGUUUUCAUtt |
| SEQ ID NO: 283 | UUACAAGGAAGUUCCAGUUtt |
| SEQ ID NO: 284 | AUUUACAAGGAAGUUCCAGtt |
| SEQ ID NO: 285 | GUUUAAAUUUACAAGGAAGtt |
| SEQ ID NO: 286 | AUUUAAACUUGGCAAUAAAtt |
| SEQ ID NO: 287 | ACUUGGCAAUAAAAGAGAAtt |
| SEQ ID NO: 288 | CUUCUAUUUAAAAGAAGUCtt |
| SEQ ID NO: 289 | ACACCACAGUCACAGAACAtt |
| SEQ ID NO: 290 | UAUGUUUGUUUUCUUCUUGtt |
| SEQ ID NO: 291 | CAUCCUCUUUGAGAACAUCtt |
| SEQ ID NO: 292 | AUUCUAUCAUCGUUCAUAA~~ |
| SEQ ID NO: 293 | CACUUUCGUUAAAACUCAA~~ |
| SEQ ID NO: 294 | AACUCAACAACCACACUGUtt |
| SEQ ID NO: 295 | CUCAACAACCACACUGUCCtt |
| SEQ ID NO: 296 | AGCAUGGUUAGAAUUGCUGtt |
| SEQ ID NO: 297 | CCAUAACUUUGAAUGUCUAtt |
| SEQ ID NO: 298 | AACUGCUGUUCCUUACUUCtt |
| SEQ ID NO: 299 | CCAGGACAAGGGAGGAGAAtt |
| SEQ ID NO: 300 | UUAGCCUGCCGGUGUCUCU~~ |
| SEQ ID NO: 301 | GAGGCGGGUGCAAAAGAGAtt |
| SEQ ID NO: 302 | AAGAGACCCAACUACAAAAtt |
| SEQ ID NO: 303 | GAAGAAGAUGAGGAGAAAGtt |
| SEQ ID NO: 304 | GAAGAUGAGGAGAAAGGGCtt |
| SEQ ID NO: 305 | GGCUAAAGGUACAGAGGGUtt |
| SEQ ID NO: 306 | AGGUACAGAGGGUGGUUCAtt |
| SEQ ID NO: 307 | GAGAUCGAGAUGGUGAUCAtt |
| SEQ ID NO: 308 | UGGAGCAGACCCUUCCUGGtt |
| SEQ ID NO: 309 | AGACUCAUCAGGAUCAUGAtt |
| SEQ ID NO: 310 | CAACGCUGCCCAGCCAGAUtt |
| SEQ ID NO: 311 | UGAAGCCCAUGAACGUGGAtt |
| SEQ ID NO: 312 | CGUGGAAUAUCUCAUGAUGtt |
| SEQ ID NO: 313 | CAGGCACGCCACUGACGGGtt |
| SEQ ID NO: 314 | GCGGCUGCCGUGUGGCGAUtt |
| SEQ ID NO: 315 | GACCCGGCGGGCCAUCCGGtt |
| SEQ ID NO: 316 | UUGCGAGGGGAGCCUGAGAtt |
| SEQ ID NO: 317 | UCGUCAACGAAACGGAAGCtt |
| SEQ ID NO: 318 | CGAAACGGAAGCAGACUCUtt |
| SEQ ID NO: 319 | ACGGAAGCAGACUCUAAGCtt |
| SEQ ID NO: 320 | CGUGGCCAGGAGCGCAGCC~~ |
| SEQ ID NO: 321 | GAGGCUGCAUGUGCUGAGCtt |
| SEQ ID NO: 322 | GCCCCACGUUGUCCUUGAAtt |
| SEQ ID NO: 323 | GCAGACUUAGAUAAACAUCtt |
| SEQ ID NO: 324 | ACAUCUCCUUUGGAUAUUUtt |
| SEQ ID NO: 325 | CAUUUAUUUUUAAAACUUC~~ |
| SEQ ID NO: 326 | AACUUCUAUUUAAAAGAAGtt |
| SEQ ID NO: 327 | GUCCAAAAACAUCAACACUtt |
| SEQ ID NO: 328 | GGUUUGAUGUCAUGUGAAAtt |
| SEQ ID NO: 329 | AAGUGUAAUAAUAACAGUU~~ |
| SEQ ID NO: 330 | GUGUAAUAAUAACAGUUAAtt |
| SEQ ID NO: 331 | UAAUAACAGUUAAGAUUUCtt |
| SEQ ID NO: 332 | UAACAGUUAAGAUUUCAUGtt |
| SEQ ID NO: 333 | CAGUUAAGAUUUCAUGAUCtt |
| SEQ ID NO: 334 | GAUUUCAUGAUCAUUUUCAtt |
| SEQ ID NO: 335 | GUACCUUCCUGGAAAGUUUtt |
| SEQ ID NO: 336 | AGAUUUCUCCAGGAAAAGG~~ |
| SEQ ID NO: 337 | AAGGAGGAAUGUAGCCAGCtt |
| SEQ ID NO: 338 | GGAGGAAUGUAGCCAGCUCtt |
| SEQ ID NO: 339 | UGUAGCCAGCUCCCCACUCtt |
| SEQ ID NO: 340 | AACCAAACAGAGACAGCUUIt |
| SEQ ID NO: 341 | CCAAAC4GAGACAGCUUCCtt |
| SEQ ID NO: 342 | ACAGAGACAGCUUCCAGCAtt |
| SEQ ID NO: 343 | AUCAAAGGAAGACACCCUCtt |
| SEQ ID NO: 344 | CGCACAGCUCAGAAAGGGGtt |
| SEQ ID NO: 345 | AGGGGGCCACAUGGGCAGAtt |
| SEQ ID NO: 346 | ACCCAAAGGAAGGACAAACtt |
| SEQ ID NO: 347 | AGGAAGGACAAACCACGAC~~ |
| SEQ ID NO: 348 | GGACAAACCACGACCACCG~~ |
| SEQ ID NO: 349 | ACCACGACCACCGUGGCCAtt |
| SEQ ID NO: 350 | ACUAGAGAAGGCAAGUGGCtt |
| SEQ ID NO: 351 | GGCGUGACCAGGAGGAACAtt |
| SEQ ID NO: 352 | GCUUUGCUUAUGUAAGGAGtt |
| SEQ ID NO: 353 | GUCAGGGAGCAGGGCUGGCtt |
| SEQ ID NO: 354 | ACCUUGGCUUUGAAUAUUGtt |
| SEQ ID NO: 355 | GGGCCAGCCUUCACCAUCG~~ |
| SEQ ID NO: 356 | AUAAAUCUGAAGCAUUUAA~~ |
| SEQ ID NO: 357 | GGCGGCUCGCGGUUCCUCCtt |
| SEQ ID NO: 358 | GACUUCCCGCAACAUCCACtt |
| SEQ ID NO: 359 | CAUCCACUCCUUGGACCACtt |
| SEQ ID NO: 360 | GUAUCUGUACCACGUUUUGtt |
| SEQ ID NO: 361 | AAACACCACAGUCACAGAAtt |
| SEQ ID NO: 362 | CAGAACCGGAACCUGCUAGtt |
| SEQ ID NO: 363 | CCGGAACCUGCUAGUGGAGtt |
| SEQ ID NO: 364 | CCUGCUAGUGGAGACCAUCtt |
| SEQ ID NO: 365 | AAUGACAGCUCUGUAUUUGtt |
| SEQ ID NO: 366 | UGACAGCUCUGUAUUUGACtt |
| SEQ ID NO: 367 | GAAUAUGUUUGUUUUCUUCtt |
| SEQ ID NO: 368 | CAUCUUGCGGCAAAAGUCGtt |
| SEQ ID NO: 369 | AAGUCGGGCCGUUACGUGUtt |
| SEQ ID NO: 370 | GUCGGGCCGUUACGUGUGCtt |
| SEQ ID NO: 371 | CAUCAGUCACGAGACCUCAtt |
| SEQ ID NO: 372 | AUAACUACGUAAAUUCUAUtt |
| SEQ ID NO: 373 | CUACGUAAAUUCUAUCAUCtt |
| SEQ ID NO: 374 | AUUUGACUUUUCUGAUGAGtt |
| SEQ ID NO: 375 | AACACUUUCGUUAAAACUC~~ |
| SEQ ID NO: 376 | CAACCACACUGUCCAUUUCtt |
| SEQ ID NO: 377 | CCACACUGUCCAUUUCUUUtt |
| SEQ ID NO: 378 | UGAGCACACCAAUGACUUUtt |
| SEQ ID NO: 379 | UGACUUUGCCCUGUACACAtt |
| SEQ ID NO: 380 | GCCAUCAAGUUUUUCAACCtt |
| SEQ ID NO: 381 | GUUUUUCAACCACCCUGAAtt |
| SEQ ID NO: 382 | CCACCCUGAAAGCAUGGUUtt |
| SEQ ID NO: 383 | UUGCUGUAAGAACCAUAAC~~ |
| SEQ ID NO: 384 | GAACCAUAACUUUGAAUGUtt |
| SEQ ID NO: 385 | CUUUGAAUGUCUAUAAAGUtt |
| SEQ ID NO: 386 | UGUCUAUAAAGUGUCAUUGtt |
| SEQ ID NO: 387 | AGUGUCAUUGGAUAACCAGtt |
| SEQ ID NO: 388 | CCAGGCCAUGCUGCACUACtt |
| SEQ ID NO: 389 | CUGCUGUUCCUUACUUCUCtt |
| SEQ ID NO: 390 | UUUGGUCUGGUUCAUUGGGtt |
| SEQ ID NO: 391 | CUCGAUGACUGCGUGCAGAtt |
| SEQ ID NO: 392 | UCGGGGUAAACUGAGUGAUtt |
| SEQ ID NO: 393 | ACUGAGUGAUCUGGUGGCAtt |
| SEQ ID NO: 394 | UGACAUCCUGAUCAUCAACtt |
| SEQ ID NO: 395 | CUGUGAGUUCCUCAACGAUtt |
| SEQ ID NO: 396 | CGAUGUGCUCACUGACCACtt |
| SEQ ID NO: 397 | CAGGCUCUUCCUGCCCCUCtt |
| SEQ ID NO: 398 | GGGAGGAGAACGGCCGAAAtt |
| SEQ ID NO: 399 | CGGCCGAAAAUUAGCCUGCtt |
| SEQ ID NO: 400 | AAUUAGCCUGCCGGUGUCUtt |
| SEQ ID NO: 401 | UUAUACAUCAUGCACCGCUtt |
| SEQ ID NO: 402 | CUCGUUAGCUGAAGUCAUUtt |
| SEQ ID NO: 403 | GUCAUUCUGAAUGGUGAUCtt |
| SEQ ID NO: 404 | UGGUGAUCUGUCUGAGAUGtt |
| SEQ ID NO: 405 | GACUGAACAGGAUAUUCAGtt |
| SEQ ID NO: 406 | CAGGAUAUUCAGAGAAGUUtt |
| SEQ ID NO: 407 | GUUCUGCCAAGCCCAGCAUtt |
| SEQ ID NO: 408 | GCCCAGCAUUCGGUGCUUCtt |
| SEQ ID NO: 409 | ACCCACCGAGACACUCGAGtt |
| SEQ ID NO: 410 | CAAGCACAAGGGCAAGAGGtt |
| SEQ ID NO: 411 | GCACAAGGGCAAGAGGCGGtt |
| SEQ ID NO: 412 | GGGCAAGAGGCGGGUGCAAtt |
| SEQ ID NO: 413 | GAGACCCAACUACAAAAACtt |
| SEQ ID NO: 414 | CUACAAAAACGUUGGGGAAtt |
| SEQ ID NO: 415 | AAACGUUGGGGAAGAAGAAtt |
| SEQ ID NO: 416 | ACGUUGGGGAAGAAGAAGAtt |
| SEQ ID NO: 417 | GAUGAGGAGAAAGGGCCCAtt |
| SEQ ID NO: 418 | AGGGCCCACCGAGGAUGCCtt |
| SEQ ID NO: 419 | GAAGACGCCGAGAAGGCUAtt |
| SEQ ID NO: 420 | GACGCCGAGAAGGCUAAAGtt |
| SEQ ID NO: 421 | AAGGCAUCAAGACGAGUGGtt |
| SEQ ID NO: 422 | GGCAUCAAGACGAGUGGGGtt |
| SEQ ID NO: 423 | GACGAGUGGGGAGAGUGAAtt |
| SEQ ID NO: 424 | GCUCUCAGAGCUGGCCGCCtt |
| SEQ ID NO: 425 | CACCACGGACGAGGAGAAAtt |
| SEQ ID NO: 426 | AAGCGCCGCCGCCACCUGCtt |
| SEQ ID NO: 427 | GCGCCGCCGCCACCUGCUCtt |
| SEQ ID NO: 428 | UAAAGGCAUGGAUCCUGAAtt |
| SEQ ID NO: 429 | AGGCAUGGAUCCUGAAAAAtt |
| SEQ ID NO: 430 | AAAUUAGAGCGAAUCCAGCtt |
| SEQ ID NO: 431 | AUUAGAGCGAAUCCAGCUCtt |
| SEQ ID NO: 432 | UCCAGCUCCCCGUGCCAAAtt |
| SEQ ID NO: 433 | AUGCGGCCGAGAAGACCACtt |
| SEQ ID NO: 434 | GACCACCUACAACCACCCGtt |
| SEQ ID NO: 435 | CCACCCGCUAGCUGAAAGAtt |
| SEQ ID NO: 436 | CGCUGCCCAGCCAGAUGGGtt |
| SEQ ID NO: 437 | GAUCCGGCUGGCGACGCUGtt |
| SEQ ID NO: 438 | GCAGCAAGUCCUGAUGAGUtt |
| SEQ ID NO: 439 | GUCCUGAUGAGUGCUGGCUtt |
| SEQ ID NO: 440 | GGACGUGCACCUGGCCUGCtt |
| SEQ ID NO: 441 | GAAAGUGUUCACCUUGUACtt |
| SEQ ID NO: 442 | AGUGUUCACCUUGUACGACtt |
| SEQ ID NO: 443 | GGGAGAAGACAUUUUUUUGtt |
| SEQ ID NO: 444 | GACAUUUUUUUGGACAUGUtt |
| SEQ ID NO: 445 | GAUGAGUAUAGGAGCAUGAtt |
| SEQ ID NO: 446 | GCCCAUGAACGUGGAAUAUtt |
| SEQ ID NO: 447 | UAUCUCAUGAUGGACGCCUtt |
| SEQ ID NO: 448 | GACUGAUGAUGUCCUGGAUtt |
| SEQ ID NO: 449 | UAACAGCGACUUGAUUGCAtt |
| SEQ ID NO: 450 | CAGCGACUUGAUUGCAUGUtt |
| SEQ ID NO: 451 | GGAUGGCGGCAUGGUCCAGtt |
| SEQ ID NO: 452 | GUUUGCAGGCCUAUUGCAGtt |
| SEQ ID NO: 453 | CAUCACCAUCCACAAGCCUtt |
| SEQ ID NO: 454 | GCCUGCGUCCAGCCCCCAUtt |
| SEQ ID NO: 455 | GCCCUUCCCCAUCCUCCAGtt |
| SEQ ID NO: 456 | GCAGCGCCUGGCCAAAGGCtt |
| SEQ ID NO: 457 | AGGCCGCAUCCAGGCAAGGtt |
| SEQ ID NO: 458 | GGCGCAUGAAGAUGCAGAGtt |
| SEQ ID NO: 459 | GAUGCAGAGAAUAGCUGCCtt |
| SEQ ID NO: 460 | UAGCUGCCCUCCUGGACCUtt |
| SEQ ID NO: 461 | UCCAGCCCACCACUGAAGUtt |
| SEQ ID NO: 462 | GUCCUGGGGUUUGGACUCGtt |
| SEQ ID NO: 463 | GGUGCCAGGCUUCGCCGUGtt |
| SEQ ID NO: 464 | ACCAGCACAGCUCCCCGUCtt |
| SEQ ID NO: 465 | CUGCCUAAGCCUCACCUUCtt |
| SEQ ID NO: 466 | GCCUCACCUUCCUGACCAGtt |
| SEQ ID NO: 467 | GCAGACUCUAAGCCCAGCAtt |
| SEQ ID NO: 468 | GCCCAGCAAGAACGUGGCCtt |
| SEQ ID NO: 469 | GAACGUGGCCAGGAGCGCAtt |
| SEQ ID NO: 470 | UCGCUGACCCUUGUCCCCCtt |
| SEQ ID NO: 471 | GACACACUGGGAGCACCCAtt |
| SEQ ID NO: 472 | CCACCUAUCCCUGCGCUCCtt |
| SEQ ID NO: 473 | UGGGAAGAAGCCCCACGUUtt |
| SEQ ID NO: 474 | GAAGCCCCACGUUGUCCUUtt |
| SEQ ID NO: 475 | UUCCUUUUUCACUUUGCAUtt |
| SEQ ID NO: 476 | UGCAGAUGACUGCACCGGCtt |
| SEQ ID NO: 477 | GAAUUGGACCAGGUCACUGtt |
| SEQ ID NO: 478 | UUGGACCAGGUCACUGUACtt |
| SEQ ID NO: 479 | AUUUGUAGAAAAGCAGACUtt |
| SEQ ID NO: 480 | AAGCAGACUUAGAUAAACAtt |
| SEQ ID NO: 481 | AAGAAGUCCAAAAACAUCAtt |
| SEQ ID NO: 482 | GAAGUCCAAAAACAUCAACtt |
| SEQ ID NO: 483 | AAACAUCAACACUAAGGUUtt |
| SEQ ID NO: 484 | ACAUCAACACUAAGGUUUGtt |
| SEQ ID NO: 485 | CACUAAGGUUUGAUGUCAUtt |
| SEQ ID NO: 486 | GUGAUUCUUUGUUACUCACtt |
| SEQ ID NO: 487 | CUCUGCAAGCCUGUGGAAUtt |
| SEQ ID NO: 488 | GCCUGUGGAAUAAUGAAGUtt |
| SEQ ID NO: 489 | UAAUGAAGUACCUUCCUGGtt |
| SEQ ID NO: 490 | UGAAGUACCUUCCUGGAAAtt |
| SEQ ID NO: 491 | AGUUUGGAUUAUUUUUUAAtt |
| SEQ ID NO: 492 | ACAAAAACAAGGGAGAUACtt |
| SEQ ID NO: 493 | AAACAAGGGAGAUACAUGUtt |
| SEQ ID NO: 494 | ACAAGGGAGAUACAUGUAUtt |
| SEQ ID NO: 495 | GGGAGAUACAUGUAUUCUCtt |
| SEQ ID NO: 496 | UGGUUUUCUGCUAUAGCAGtt |
| SEQ ID NO: 497 | GAAGGAACCAGUUGGGACC~~ |
| SEQ ID NO: 498 | GGAACCAGUUGGGACCGUGtt |
| SEQ ID NO: 499 | CCAGUUGGGACCGUGAAGAtt |
| SEQ ID NO: 500 | GACUCCCGACCCUGUGGCCtt |
| SEQ ID NO: 501 | AGGAAGACACCCUCUACGUtt |
| SEQ ID NO: 502 | GACACCCUCUACGUCACCUtt |
| SEQ ID NO: 503 | GGGCAUGGUGUCCCCAGAGtt |
| SEQ ID NO: 504 | UAUGGAAACCCCAUGGUUCtt |
| SEQ ID NO: 505 | ACCCCAUGGUUCCCUUCCCtt |
| SEQ ID NO: 506 | GCAAGGACCCAGAUGCAUCtt |
| SEQ ID NO: 507 | GGACCCAGAUGCAUCAGACtt |
| SEQ ID NO: 508 | GAUGUUCCUUUCUACUCGGtt |
| SEQ ID NO: 509 | GUCCACCAGGGCCAGCGGCtt |
| SEQ ID NO: 510 | CUUUCCUCCAGACCUGCCAtt |
| SEQ ID NO: 511 | GAACAUGGUCUCUGUCUCCtt |
| SEQ ID NO: 512 | CAUGGUCUCUGUCUCCUCGtt |
| SEQ ID NO: 513 | GGCCUGCCGAGGGCAGUUUtt |
| SEQ ID NO: 514 | CCUCAUGAAGGAAACACAGtt |
| SEQ ID NO: 515 | GGAAACACAGUCCUGCCAAtt |
| SEQ ID NO: 516 | ACACAGUCCUGCCAAGGAGtt |
| SEQ ID NO: 517 | GGAGGGGGAGUGGCGCCCAtt |
| SEQ ID NO: 518 | GCCCUCUGGGUAGCUGUGCtt |
| SEQ ID NO: 519 | GGACCUUUGCUUCCAUAGAtt |
| SEQ ID NO: 520 | AACGCACAGCUCAGAAAGGtt |
| SEQ ID NO: 521 | UCCCUGGAAGAGAAGGAAGtt |
| SEQ ID NO: 522 | GAGAAGGAAGGCAGGGUGGtt |
| SEQ ID NO: 523 | GGAAGGCAGGGUGGAGCGGtt |
| SEQ ID NO: 524 | GGCAGGGUGGAGCGGGGGGtt |
| SEQ ID NO: 525 | GACCAUCAUGGAGAGAAGGtt |
| SEQ ID NO: S26 | GGACCACAGCAUCAGGAGAtt |
| SEQ ID NO: 527 | UUUUUUAAGAGCAAGAGGGtt |
| SEQ ID NO: 528 | GAGCAAGAGGGGUAGAGAGtt |
| SEQ ID NO: 529 | GAGGGGUAGAGAGGAUCAAtt |
| SEQ ID NO: 530 | GCUGGCCCUGGCUGGAGAUtt |
| SEQ ID NO: 531 | AUGACUCUGUCUGUGGGGCtt |
| SEQ ID NO: 532 | GGCAAGUGGCUGCCCCACCtt |
| SEQ ID NO: 533 | GUGGCUGCCCCACCCCAAGtt |
| SEQ ID NO: 534 | CAGCCUGCAGCUCACUCCAtt |
| SEQ ID NO: 535 | GUCUCUGGGCUCCAACUGGtt |
| SEQ ID NO: 536 | CUGGUCUUGUAACCACUGAtt |
| SEQ ID NO: 537 | CCACUGAGCACUGAAGGAGtt |
| SEQ ID NO: 538 | GGAGAGAGGUCUUGGUCAGtt |
| SEQ ID NO: 539 | AGGUGACUGGGAGGACCAGtt |
| SEQ ID NO: 540 | AAGACACUGCUCAGGGCAGtt |
| SEQ ID NO: 541 | GACACUGCUCAGGGCAGGGtt |
| SEQ ID NO: 542 | GAAAAGGGCAGUCCCCAUGtt |
| SEQ ID NO: 543 | AAGGGCAGUCCCCAUGUGGtt |
| SEQ ID NO: 544 | GGGCAGUCCCCAUGUGGGCtt |
| SEQ ID NO: 545 | CUUGCGUCUGGGGGACACCtt |
| SEQ ID NO: 546 | CCAGGGCUCCCAGAAGCUUtt |
| SEQ ID NO: 547 | GGAGGUCUGGGAGCCAGCCtt |
| SEQ ID NO: 548 | AGGACCCCACCUCACCCAGtt |
| SEQ ID NO: 549 | ACCUCAUAGCUGGGGCGCUtt |
| SEQ ID NO: 550 | GAGCCAGGAGUGUGGGAAGtt |
| SEQ ID NO: 551 | GGCCCACAGUGGGGGCUGUtt |
| SEQ ID NO: 552 | GUCCCUGCAGAGAGCACUUtt |
| SEQ ID NO: 553 | CUCCCAAACCUUGGCUUUGtt |
| SEQ ID NO: 554 | UAUUGUUGUGGAGGUGUGCtt |
| SEQ ID NO: 555 | GGUACCUGUCCCAGCAGGUtt |
| SEQ ID NO: 556 | CCCAGACACUUGCGUUCACtt |
| SEQ ID NO: 557 | GCAACCUAAGGGGCAGGUGtt |
| SEQ ID NO: 558 | CCUAAGGGGCAGGUGAAGA~ |
| SEQ ID NO: 559 | GGGGCAGGUGAAGAAGCGCtt |
| SEQ ID NO: 560 | GAAGCGCAGCCCUGCCAGAtt |
| SEQ ID NO: 561 | GCGCAGCCCUGCCAGACGCtt |
| SEQ ID NO: 562 | GGUCUCUGAGAUGCACCGUtt |
| SEQ ID NO: 563 | AAAGGCGUGGGGUGAACUGtt |
| SEQ ID NO: 564 | AGGCGUGGGGUGAACUGAUtt |
| SEQ ID NO: 565 | CUGAUUUUGAUCUUCUUGUtt |
| SEQ ID NO: 566 | UAAAUAAAUCUGAAGCAUUtt |
| SEQ ID NO: 567 | AUCUGAAGCAUUUAAUGUAtt |
| SEQ ID NO: 568 | GCAUUUAAUGUAGUCAUCUtt |
| SEQ ID NO: 569 | UGUAGUCAUCUUGACAUUGtt |
| SEQ ID NO: 570 | AAAUAUGUAAAUAAUUUUUIt |
| SEQ ID NO: 571 | AUAUGUAAAUAAUUUUUGUtt |
| SEQ ID NO: 572 | AUAAUUUUUGUCCCAGUGAtt |
| SEQ ID NO: 573 | UUUUUGUCCCAGUGAGAACtt |
| SEQ ID NO: 574 | CCGAGGGUUAGAAAACCUCtt |
| SEQ ID NO: 575 | AACCUCGAUGCCUCUGAGCtt |
| SEQ ID NO: 576 | CCUCGAUGCCUCUGAGCCUtt |
| SEQ ID NO: 577 | GUACCUGCUUUCGCCAGCAtt |
| SEQ ID NO: 578 | CACGAGGGUGGAAAUGAAAtt |
| SEQ ID NO: 579 | AUGAAAACUGGAACUUCCUtt |
| SEQ ID NO: 580 | AACUGGAACUUCCUUGUAAtt |
| SEQ ID NO: 581 | CUGGAACUUCCUUGUAAAUtt |
| SEQ ID NO: 582 | CUUCCUUGUAAAUUUAAACtt |

Agents which reduce CLEC16A expression are not limited to siRNAs. Any agent that partially reduces CLEC16A expression is within the scope of the present invention.

### REFERENCES

Fenner, J. E., Starr, R., Cornish, A. L., Zhang, J. G., Metcalf, D., Schreiber, R. D., Sheehan, K., Hilton, D. J., Alexander, W. S., and Hertzog, P. J. (2006). Suppressor of cytokine signaling 1 regulates the immune response to infection by a unique inhibition of type I interferon activity. Nat Immunol 7, 33-39.
Khaibullin, T., Ivanova, V., Martynova, E., Cherepnev, G., Khabirov, F., Granatov, E., Rizvanov, A., and Khaiboullina, S. (2017). Elevated Levels of Proinflammatory Cytokines in Cerebrospinal Fluid of Multiple Sclerosis Patients. Front Immunol 8, 531.
Leikfoss, I. S., Mero, I. L., Dahle, M. K., Lie, B. A., Harbo, H. F., Spurkland, A., and Berge, T. (2013). Multiple sclerosis-associated single-nucleotide polymorphisms in CLEC16A correlate with reduced SOCS1 and DEXI expression in the thymus. Genes Immun 14, 62-66.
Redmann, V., Lamb, C. A., Hwang, S., Orchard, R. C., Kim, S., Razi, M., Milam, A., Park, S., Yokoyama, C. C., Kambal, A., et al. (2016). Clec16a is Critical for Autolysosome Function and Purkinje Cell Survival. Sci Rep 6, 23326.
Soleimanpour, S. A., Gupta, A., Bakay, M., Ferrari, A. M., Groff, D. N., Fadista, J., Spruce, L. A., Kushner, J. A., Groop, L., Seeholzer, S. H., et al. (2014). The diabetes susceptibility gene Clec16a regulates mitophagy. Cell 157, 1577-1590.
Tam, R. C., Li, M. W., Gao, Y. P., Pang, Y. T., Yan, S., Ge, W., Lau, C. S., and Chan, V. S. (2017). Human CLEC16A regulates autophagy through modulating mTOR activity. Exp Cell Res 352, 304-312.

Huang M, Verbeek D S. (2018). Why do so many genetic insults lead to Purkinje Cell degeneration and spinocerebellar ataxia? Neurosci Lett. 2018 Feb. 5. pii: S0304-3940(18)30080-6

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A method of management for obesity in a subject in need thereof comprising administering to said subject a therapeutic agent in amount effective to partially reduce CLEC16A expression, thereby managing or reducing obesity.

2. The method of claim 1, wherein said therapeutic agent modulates signaling mediated via the CLEC16A gene product.

3.-6. (canceled)

7. The method of claim 1, wherein said therapeutic agent is at least one siRNA molecule is set forth in Table 1.

8. The method of claim 1, wherein said therapeutic agent is delivered to an adipose cell.

9. The method of claim 1, wherein said therapeutic agent modulates natural killer cell activity.

10. The method of claim 1, wherein said therapeutic agent modulates signaling in an insulin-producing beta cell.

11. An siRNA composition comprising at least one nucleotide sequence selected from the group listed in Table 1 in a pharmaceutically acceptable carrier for delivery to a patient.

12. A method of partially inhibiting the expression of CLEC16A in a patient comprising administering to said patient at least one siRNA molecule that directs cleavage of a target CLEC16A mRNA sequence present in said patient.

13. The method of claim 12, wherein said siRNA is introduced directly into said patient.

14. The method of claim 7, wherein the siRNA is combined with other agents for treating obesity.

15. The method of claim 7, further comprising administration of a Jak-Stat inhibitor.

16. The method of claim 7, further comprising administration of a mTor inhibitor.

17. The method of claim 12, further comprising administration of a Jak-Stat inhibitor.

18. The method of claim 12, further comprising administration of a mTor inhibitor.

19. The method of claim 12, further comprising administration of an autophagy inhibitor.

20. The method of claim 7, further comprising administration of an autophagy inhibitor.
